(19) United States
(12) Patent Application Publication
(10) Pub. No.: US 2023/0016396 A1
(43) Pub. Date: Jan. 19, 2023

(54) METHODS OF POLYPEPTIDE SEQUENCING

(71) Applicant: ENCODIA, INC., San Diego, CA (US)

(72) Inventors: Kevin L. GUNDERSON, San Diego, CA (US); Norihito MURANAKA, San Diego, CA (US); Lei SHI, San Diego, CA (US); Stephen VERESPY, III, San Diego, CA (US)

(73) Assignee: ENCODIA, INC., San Diego, CA (US)

(21) Appl. No.: 17/848,010

(22) Filed: Jun. 23, 2022

Related U.S. Application Data
(60) Provisional application No. 63/215,867, filed on Jun. 28, 2021.

(51) Int. Cl. G01N 33/68; G01N 33/58; C12N 9/48

(52) U.S. Cl. CPC G01N 33/6824; G01N 33/582; C12N 9/485

(57) ABSTRACT

The present disclosure relates to methods and kits for performing an identification of a terminal amino acid residue of the polypeptide, or performing a polypeptide sequencing. The methods include a step of contacting the terminal amino acid residue of the polypeptide with a coupler, followed by attaching the coupler-polypeptide complex to the solid support and cleaving the coupler-polypeptide complex from the polypeptide, thereby isolating the terminal amino acid residue of the polypeptide from the remaining amino acid residues of the polypeptide in complex with the coupler, thereby enabling efficient identification of the terminal amino acid residue via recognition by binding agents capable of binding to the coupler-amino acid complex. In some embodiments, the coupler and the polypeptide are both associated with stabilizing components, and after binding of the coupler to the terminal amino acid of the polypeptide, tethering complex is formed between the stabilizing components releasably attached to the solid support.

### CROSS-REFERENCE TO RELATED APPLICATIONS

The present application claims priority to U.S. provisional patent application No. 63/215,867, filed on Jun. 28, 2021, the disclosure and content of which are incorporated herein by reference in its entirety for all purposes.

### SEQUENCE LISTING ON ASCII TEXT

This patent or application file contains a Sequence Listing submitted in computer readable ASCII text format (file name: 4614-2003400_SeqList.txt, date recorded: Jun. 22, 2022, size: 50,099 bytes). The content of the Sequence Listing file is incorporated herein by reference in its entirety.

### TECHNICAL FIELD

The present disclosure relates generally to biotechnology, in particular to methods for sequencing of polypeptides employing single amino acid recognizing reagents and methods of single amino acid isolation. In some embodiments, the present disclosure finds utility in a variety of methods and related kits for high-throughput polypeptides sequencing.

### BACKGROUND

Recognition and binding of molecular targets using binding agents can be useful for characterization, detection and/or analysis of target biomolecules, such as polypeptides. Previously known methods include various immunoassay formats, such as ELISA, or use of mass spectrometry (e.g., Smits et al., Trends Biotechnol. (2016) 34(10):825-834; O'Reilly et al., Nat Struct Mol Biol. (2018) 25(11):1000-1008). Recently, methods have been disclosed that utilize use of binding agents for high-throughput polypeptide sequencing, for example, U.S. Pat. No. 9,435,810 B2, WO2010065531A1, US 20190145982 A1, US 20200217853 A1, US 20200348308 A1, US 20200400677 A1. These methods utilize N-terminal amino acid (NTAA) recognition by binding agents as a critical step in a polypeptide sequencing assay. A number of methods to evolve specific NTAA binding agents from different scaffolds for recognizing a particular terminal amino acid have also been proposed, including directed evolution approaches to derive amino acyl tRNA synthetases, N-recognins such as ClpS and ClpS2, anticalins, and aminopeptidases, which are disclosed, for example, in US 20190145982 A1 and U.S. Pat. No. 9,435,810 B2. However, identifying binding agents that afford amino acid specificity with sufficiently strong affinity has proven challenging. Binding affinity and/or specificity towards a terminal amino acid residue (P1) can vary depending on neighboring amino acids of the polypeptide to be analyzed, e.g. the penultimate terminal amino acid residue (P2) and the antepenultimate amino acid residue (P3). In some cases, crosslinking reagents and methods exist for applications involving binding agents recognizing polypeptides. It may be preferred that binding agents and detection assays are performed in a manner that allows specificity and stability in a controllable manner that allows processing of a plurality of binding agents and polypeptides at the same time. Additionally, speed and reversibility may also be a desired feature for these binding reactions. However, current reagents and methods are somewhat limited in these aspects.

The present disclosure describes novel and improved approaches for performing terminal amino acid residue recognition by binding agent(s), followed by identification of the terminal amino acid residue of the polypeptide. These approaches address a need for proteomics technology that is highly-parallelized, accurate, sensitive, and/or high-throughput. These and other aspects of the disclosure will be apparent upon reference to the following detailed description. To this end, various references are set forth herein which describe in more detail certain background information, procedures, compounds and/or compositions, and are each hereby incorporated by reference in their entireties.

### BRIEF SUMMARY

The summary is not intended to be used to limit the scope of the claimed subject matter. Other features, details, utilities, and advantages of the claimed subject matter will be apparent from the detailed description including those aspects disclosed in the accompanying drawings and in the appended claims.

The present disclosure relates to methods and kits for performing an identification of a terminal amino acid residue of a polypeptide, or identification of a portion of the polypeptide sequence. The methods include a step of contacting the terminal amino acid residue of the polypeptide with a coupler, followed by attaching the coupler-polypeptide complex to a solid support and cleaving the coupler-polypeptide complex from the polypeptide, thereby isolating the terminal amino acid residue of the polypeptide from the remaining amino acid residues of the polypeptide in complex with the coupler, thereby enabling efficient identification of the terminal amino acid residue via recognition by binding agent(s) capable of binding to the coupler-amino acid complex. The identification occurs via transferring information about the binding agent to a recording tag followed by analysis of the recording tag. In some embodiments, the coupler and the polypeptide are both associated with stabilizing components, and after binding of the coupler to the terminal amino acid of the polypeptide, a tethering complex is formed between the stabilizing components releasably attaching the terminal amino acid to a solid support for the following identification. The provided tethering reaction ensures a controllable way of attaching the coupler-amino acid complex to the solid support. After the information transfer regarding the binding agent bound to the coupler-amino acid complex, the attachment can be efficiently reversed by releasing or disrupting the tethering complex, allowing for repeating the identification cycle by contacting the coupler with newly formed terminal amino acid residue of the polypeptide. Other attachment ways of the coupler-amino acid complex are also possible. In preferred embodiments, identification occurs in parallel for multiple polypeptides with the help of multiple binding agents, wherein each binding agent is specific for a particular amino acid in complex with the coupler. This approach allows for selection of specific high affinity binding agents, since their affinity towards a particular amino acid does not depend on neighboring amino acid residues. Moreover, the described information transfer allows for utilizing next generation sequencing platforms for recording tag analysis and amino acid identification, which results in a highly parallel, high throughput approach for polypeptide sequencing. Also provided are kits containing components and/or reagents for performing the provided tethering reactions. In some embodiments, the kits also include instructions for performing any of the methods provided for performing tethering reactions and polypeptide sequencing.

In one embodiment, provided herein is a method for identifying a terminal amino acid of a polypeptide, comprising the steps of: (a) providing a polypeptide and an associated recording tag attached to a solid support; (b) contacting the polypeptide with a coupler, wherein the coupler binds to a terminal amino acid of the polypeptide to form a coupler-polypeptide complex; (c) attaching the coupler to the solid support; (d) cleaving the peptide bond between the terminal amino acid and the penultimate terminal amino acid of the polypeptide within the coupler-polypeptide complex to generate a coupler-amino acid complex attached to the solid support; (e) contacting the coupler-amino acid complex with a binding agent capable of binding to the coupler-amino acid complex, wherein the binding agent comprises a coding tag with identifying information regarding the binding agent; (f) transferring the information of the coding tag of the binding agent to the recording tag to generate an extended recording tag; and (g) analyzing the extended recording tag, thereby identifying the terminal amino acid of the polypeptide.

In another embodiment, a method for identifying at least a portion of a sequence of a polypeptide is provided, comprising the steps of: (a) providing a polypeptide and an associated recording tag attached to a solid support; (b) contacting the polypeptide with a coupler, wherein the coupler binds to a terminal amino acid of the polypeptide to form a coupler-polypeptide complex; (c) attaching the coupler to the solid support; (d) cleaving the peptide bond between the terminal amino acid and the penultimate terminal amino acid of the polypeptide within the coupler-polypeptide complex, thereby exposing a new terminal amino acid of the polypeptide and generating a coupler-amino acid complex attached to the solid support; (e) contacting the coupler-amino acid complex attached to the solid support with a binding agent capable of binding to the coupler-amino acid complex, wherein the binding agent comprises a coding tag with identifying information regarding the binding agent; (f) transferring the information of the coding tag of the binding agent to the recording tag to generate an extended recording tag; (g) releasing the coupler-amino acid complex from the solid support; (h) repeating steps (b) through (g) at least one more time; (i) analyzing the extended recording tag after information transfer, thereby identifying at least a portion of the sequence of the polypeptide.

In yet another embodiment, a method for identifying a terminal amino acid of a polypeptide is provided, comprising: (a) providing a polypeptide and an associated recording tag attached to a solid support; (b) providing a first stabilizing component associated with the polypeptide; (c) contacting the polypeptide with a coupler, wherein the coupler binds to a terminal amino acid of the polypeptide to form a coupler-polypeptide complex, and wherein the coupler is attached to a second stabilizing component; (d) after binding of the coupler to the terminal amino acid of the polypeptide, linking the first and second stabilizing components together to form a tethering complex between the first stabilizing component attached to the solid support and the second stabilizing component linked to the coupler-polypeptide complex, wherein the first and second stabilizing components can bind each other, or can both bind a linking agent, such as a linking polypeptide; (e) cleaving the peptide bond between the terminal amino acid and the penultimate terminal amino acid of the polypeptide within the coupler-polypeptide complex to generate a coupler-amino acid complex attached to the solid support via the tethering complex; (f) contacting the coupler-amino acid complex with a binding agent capable of binding selectively to the coupler-amino acid complex, wherein the binding agent comprises a coding tag with identifying information regarding the binding agent; (g) transferring the information of the coding tag of the binding agent to the recording tag to generate an extended recording tag; (h) analyzing the extended recording tag after information transfer, thereby identifying the terminal amino acid of the polypeptide.

In some embodiments of the previous method, step (b) is performed before step (c). In other embodiments of the previous method, step (b) is performed after step (c) or together with step (c).

In yet another embodiment, a method for identifying at least a portion of a sequence of a polypeptide is provided, comprising: (a) providing a polypeptide and an associated recording tag attached to a solid support; (b) providing a first stabilizing component associated with the polypeptide; (c) contacting the polypeptide with a coupler, wherein the coupler binds to a terminal amino acid of the polypeptide to form a coupler-polypeptide complex, and wherein the coupler is attached to a second stabilizing component; (d) after binding of the coupler to the terminal amino acid of the polypeptide, linking the first and second stabilizing components together to form a tethering complex between the first stabilizing component attached to the solid support and the second stabilizing component linked to the coupler-polypeptide complex, wherein the first and second stabilizing components can bind each other, or can both bind a linking agent, such as a linking polypeptide; (e) cleaving the peptide bond between the terminal amino acid and the penultimate terminal amino acid of the polypeptide within the coupler-polypeptide complex to generate a coupler-amino acid complex attached to the solid support via the tethering complex; (f) contacting the coupler-amino acid complex with a binding agent capable of binding to the coupler-amino acid complex, wherein the binding agent comprises a coding tag with identifying information regarding the binding agent; (g) transferring the information of the coding tag of the binding agent to the recording tag to generate an extended recording tag; (h) releasing the coupler-amino acid complex from the solid support; (i) repeating steps (c) through (h) or steps (b) through (h) at least one more time; (j) analyzing the extended recording tag after information transfer, thereby identifying at least a portion of the sequence of the polypeptide.

In some embodiments of the previous method, step (b) is performed before step (c). In other embodiments of the previous method, step (b) is performed after step (c) or together with step (c).

In yet another embodiment, a kit for analyzing a polypeptide is provided, comprising: a coupler, wherein the coupler is configured to bind to a terminal amino acid of the polypeptide to form a coupler-polypeptide complex; a reagent for cleaving the peptide bond between the terminal amino acid and the penultimate terminal amino acid of the polypeptide within the coupler-polypeptide complex to generate a coupler-amino acid complex; and a binding agent capable of binding to the coupler-amino acid complex.

### BRIEF DESCRIPTION OF THE DRAWINGS

Non-limiting embodiments of the present invention will be described by way of example with reference to the accompanying figures, which are schematic and are not intended to be drawn to scale. For purposes of illustration, not every component is labeled in every figure, nor is every component of each embodiment of the invention shown where illustration is not necessary to allow those of ordinary skill in the art to understand the invention. While the drawings depict stabilizing components and linking agents each as one molecule, it is within the scope of the invention that each stabilizing component or the linking agent may contain sub-components or comprise two or more molecules.

The following labeling is used in the Figures: 1—recording tag; 2 capture DNA; 3—polypeptide; 4—the NTAA of the polypeptide; 5—solid support; 6—first stabilizing component; 7—second stabilizing component; 8—the NTM group of the coupler; 9—linking agent; 10—binding agent; 11—coding tag.

FIG. 1A-FIG. 1E depict an exemplary method for identifying a terminal amino acid of a polypeptide, comprising steps of contacting the polypeptide 3 with a coupler (NTAA modification), tethering, cleavage, and encoding (information transfer). In FIG. 1A, a DNA-polypeptide conjugate is immobilized on a solid support 5. A first stabilizing component 6 is shown attached near the 5′ end of the capture DNA 2 comprising the recording tag 1. A polypeptide 3 is attached near the 3′ end of the capture DNA 2. FIG. 1B shows an N-terminal functionalization step, where attachment of a coupler 8 to the NTAA of the polypeptide 4 occurs. The coupler also contains a linker region and a second stabilizing component 7 attached to the linker. In FIG. 1C, the coupler-polypeptide is exposed to a linking agent 9 which tethers the coupler to the 5′ end of the capture DNA 2 by binding and bringing the first and second stabilizing components together. In FIG. 1D, the polypeptide-DNA conjugate with the tethered coupler is exposed to a cleavage agent which cleaves the coupler-NTAA complex from the polypeptide. The carboxyl moiety of the cleaved and tethered coupler-NTAA complex is now exposed for interaction with the binding agent 10 and subsequent identification. In FIG. 1E, the NTAA is identified with a binding agent that selectively recognizes the coupler-NTAA moiety; an encoding step transfers information from the binding agent-associated DNA coding tag 11 to the DNA recording tag 1.

FIG. 2A-FIG. 2F depicts an exemplary method for identifying a terminal amino acid of a polypeptide, comprising steps of contacting the polypeptide 3 with a coupler (NTAA modification), tethering, cleavage, and encoding (information transfer) using stabilizing components attached to the capture DNA via DNA hybridization. In FIG. 2A, a DNA-polypeptide conjugate is immobilized on a solid support 5. A polypeptide is attached near the 3′ end of the capture DNA 2 comprising the recording tag 1. FIG. 2B shows an N-terminal functionalization step, where attachment of a coupler 8 to the NTAA of the polypeptide 4 occurs. The coupler also contains a linker region and a second stabilizing component 7 attached to the linker. In FIG. 2C, a first stabilizing component 6 is hybridized to its cognate sequence located at the 5′ end of the capture DNA 2 comprising the recording tag 1. In FIG. 2D, the coupler-polypeptide is exposed to a linking agent 9 which tethers the coupler to the 5′ end of the capture DNA 2 by binding and linking the first and second stabilizing components together. In FIG. 2E, the polypeptide-DNA conjugate with the tethered coupler is exposed to a cleavage agent which cleaves the coupler-NTAA complex from the polypeptide. The carboxyl (COOH) moiety of the cleaved and tethered coupler-NTAA is now exposed for interaction with the binding agent 10 and subsequent identification. In FIG. 2F, the NTAA is identified with a binding agent that selectively recognizes the coupler-NTAA moiety; an encoding step transfers information from the binding agent-associated DNA coding tag 11 to the DNA recording tag 1. The stabilizing components, bound binding agent with the coding tag, and the tethered coupler-NTAA can be removed using heating or alkaline stripping conditions (e.g. 0.1 NaOH). After stripping, the system is ready for the next cycle of NTAA identification.

FIG. 3 depicts exemplary embodiments of a controllable tethering reaction between two stabilizing components each comprising a polynucleotide. Light or a linking agent can trigger isomerization, uncaging or structural transformation of one of the stabilizing components that can result in DNA hybridization and association between the components or between the component(s) and the linking agent. In some cases, the linking agent comprises a polypeptide.

FIG. 4 shows exemplary coupler reagent for NTAA labeling and cleavage of the immobilized polypeptide analyte. R1 and R2 represent side chains of NTAA residue and penultimate terminal amino acid residue of the peptide analyte, respectively. R1 and R2 are independently selected from the group consisting of: CH3-, HN═C(NH2)-NH—(CH2)3-, H2N—CO—CH2-, HOOC—CH2-, HS—CH2-, H2N—CO—(CH2)2-, HOOC—(CH2)2-, H—, NH—CH═N—CH═C—CH2-, CH3-CH2-CH(CH3)-, (CH3)2-CH—CH2-, H2N—(CH2)4-, CH3-S—(CH2)2-, Ph-CH2-, HO—CH2-, CH3-CH(OH)—, Ph-NH—CH═C—CH2-, HO-Ph-CH2-, and (CH3)2-CH—.

FIG. 5 shows multiple sequence alignment of the active Carboxypeptidase A and Carboxypeptidase T gene sequences (pre-pro peptide sequence was excluded from alignment) from different organisms as potential scaffolds for selective binding agents. The alignment is produced using Clustal Omega multiple sequence alignment tool from the European Bioinformatics Institute (EMBL-EBI). SEQ ID NO:1-5 are shown. A residue 255 is highlighted in the alignment, which is crucial for conferring specificity towards particular C-terminal residue.

FIG. 6 depicts an exemplary structure of the coupler and its interaction with the polypeptide immobilized on a solid support.

FIG. 7A illustrates exemplary cleavages of M15-L-modified NTAAs of a model polypeptide (M15-L-P1-AR) by engineered dipeptidyl peptidase enzymes. A compilation of seven different modified dipeptidyl peptidase clones was used to generate the spectrum of cleavage profiles across all 20 M15-L-modified NTAAs as shown. Data were generated by HPLC analysis (UV absorbance) of cleaved versus intact peptides after the cleavase assay.

FIG. 7B shows cleavage events on peptides attached to DNA tag using gel-shift analysis on an SDS-PAGE gel. FIG. 7C shows a cleavage profile for an exemplary set of two selected engineered dipeptidyl peptidase clones, M15-L_Z001, having specificity towards A, I, L, M, Q, V in the P1 position (cleavage efficiency of M15-L_Z001 is shown by the left columns for each amino acid), and M15-L_Z002, having specificity towards D and E in the P1 position (cleavage efficiency of M15-L_Z002 is shown by the right columns for each amino acid).

FIG. 8A-FIG. 8B show the results of an exemplary cleavage reaction to evaluate activity of an engineered dipeptidyl peptidase mutant on a NTM-modified peptide (M15-K(biotin) attached to an AAR peptide). A designates a signal from M15-K(biotin)-AAR peptide; B designates a signal from M15-K(biotin)-A molecule, and C designates a signal from a control peptide. The UV absorbance of both starting material (FIG. 8A) and reaction product (FIG. 8B) was measured on HPLC.

### DETAILED DESCRIPTION

Numerous specific details of the provided methods and kits are set forth in the following description in order to provide a thorough understanding of the present disclosure. These details are provided for the purpose of example and the claimed subject matter may be practiced according to the claims without some or all of these specific details. It is to be understood that other embodiments can be used and structural changes can be made without departing from the scope of the claimed subject matter. It should be understood that the various features and functionality described in one or more of the individual embodiments are not limited in their applicability to the particular embodiment with which they are described. They instead can be applied, alone or in some combination, to one or more of the other embodiments of the disclosure, whether or not such embodiments are described, and whether or not such features are presented as being a part of a described embodiment. For the purpose of clarity, technical material that is known in the technical fields related to the claimed subject matter has not been described in detail so that the claimed subject matter is not unnecessarily obscured.

All publications, including patent documents, scientific articles and databases, referred to in this application are incorporated by reference in their entireties for all purposes to the same extent as if each individual publication were individually incorporated by reference. Citation of the publications or documents is not intended as an admission that any of them is pertinent prior art, nor does it constitute any admission as to the contents or date of these publications or documents.

All headings are for the convenience of the reader and should not be used to limit the meaning of the text that follows the heading, unless so specified.

### Definitions

Unless defined otherwise, all technical and scientific terms used herein have the same meaning as is commonly understood by one of ordinary skill in the art to which the present disclosure belongs. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, the singular forms “a,” “an” and “the” include plural referents unless the context clearly dictates otherwise. Thus, for example, reference to “a peptide” includes one or more peptides, or mixtures of peptides. Also, and unless specifically stated or obvious from context, as used herein, the term “or” is understood to be inclusive and covers both “or” and “and”.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

An “individual” or “subject” includes a mammal. Mammals include, but are not limited to, domesticated animals (e.g., cows, sheep, cats, dogs, and horses), primates (e.g., humans and non-human primates such as monkeys), rabbits, and rodents (e.g., mice and rats).

As used herein, the term “sample” refers to anything which may contain an analyte for which an analyte assay is desired. As used herein, a “sample” can be a solution, a suspension, liquid, powder, a paste, aqueous, non-aqueous or any combination thereof. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregate of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s). In some embodiments, the sample is a biological sample. A biological sample of the present disclosure encompasses a sample in the form of a solution, a suspension, a liquid, a powder, a paste, an aqueous sample, or a non-aqueous sample. As used herein, a “biological sample” includes any sample obtained from a living or viral (or prion) source or other source of polypeptides and biomolecules, and includes any cell type or tissue of a subject from which nucleic acid, protein and/or other polypeptide can be obtained. The biological sample can be a sample obtained directly from a biological source or a sample that is processed.

The terms “level” or “levels” are used to refer to the presence and/or amount of a target, e.g., a substance or an organism that is part of the etiology of a disease or disorder, and can be determined qualitatively or quantitatively. A “qualitative” change in the target level refers to the appearance or disappearance of a target that is not detectable or is present in samples obtained from normal controls. A “quantitative” change in the levels of one or more targets refers to a measurable increase or decrease in the target levels when compared to a healthy control.

As used herein, the term “polypeptide” encompasses large molecules composed of smaller subunits. Examples of polypeptides include, but are not limited to peptides, polypeptides, proteins, nucleic acids, carbohydrates, lipids, macrocycles, or a combination or complex thereof. A polypeptide also includes a chimeric polypeptide composed of a combination of two or more types of polypeptides, covalently linked together (e.g., a peptide linked to a nucleic acid). A polypeptide may also include a “polypeptide assembly”, which is composed of non-covalent complexes of two or more polypeptides. A polypeptide assembly may be composed of the same type of polypeptide (e.g., protein-protein) or of two or more different types of polypeptides (e.g., protein-DNA).

As used herein, the term “polypeptide” encompasses peptides and proteins, and refers to a molecule comprising a chain of two or more amino acids joined by peptide bonds. In some embodiments, a polypeptide comprises 2 to 50 amino acids, e.g., having more than 20-30 amino acids. In some embodiments, a peptide does not comprise a secondary, tertiary, or higher structure. In some embodiments, the polypeptide is a protein. In some embodiments, a protein comprises 30 or more amino acids, e.g. having more than 50 amino acids. In some embodiments, in addition to a primary structure, a protein comprises a secondary, tertiary, or higher structure. The amino acids of the polypeptides are most typically L-amino acids, but may also be D-amino acids, modified amino acids, amino acid analogs, amino acid mimetics, or any combination thereof. Polypeptides may be naturally occurring, synthetically produced, or recombinantly expressed. Polypeptides may be synthetically produced, isolated, recombinantly expressed, or be produced by a combination of methodologies as described above. Polypeptides may also comprise additional groups modifying the amino acid chain, for example, functional groups added via post-translational modification. The polymer may be linear or branched, it may comprise modified amino acids, and it may be interrupted by non-amino acids. The term also encompasses an amino acid polymer that has been modified naturally or by intervention; for example, disulfide bond formation, glycosylation, lipidation, acetylation, phosphorylation, or any other manipulation or modification, such as conjugation with a labeling component.

As used herein, the term “amino acid” refers to an organic compound comprising an amine group, a carboxylic acid group, and a side-chain specific to each amino acid, which serve as a monomeric subunit of a peptide. An amino acid includes the 20 standard, naturally occurring or canonical amino acids as well as non-standard amino acids. The standard, naturally-occurring amino acids include Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). An amino acid may be an L-amino acid or a D-amino acid. Non-standard amino acids may be modified amino acids, amino acid analogs, amino acid mimetics, non-standard proteinogenic amino acids, or non-proteinogenic amino acids that occur naturally or are chemically synthesized. Examples of non-standard amino acids include, but are not limited to, selenocysteine, pyrrolysine, and N-formylmethionine, R-amino acids, Homo-amino acids, Proline and Pyruvic acid derivatives, 3-substituted alanine derivatives, glycine derivatives, ring-substituted phenylalanine and tyrosine derivatives, linear core amino acids, N-methyl amino acids.

As used herein, the term “post-translational modification” refers to modifications that occur on a peptide after its translation, e.g., translation by ribosomes, is complete. A post-translational modification may be a covalent chemical modification or enzymatic modification. Examples of post-translation modifications include, but are not limited to, acylation, acetylation, alkylation (including methylation), biotinylation, butyrylation, carbamylation, carbonylation, deamidation, deiminiation, diphthamide formation, disulfide bridge formation, eliminylation, flavin attachment, formylation, gamma-carboxylation, glutamylation, glycylation, glycosylation, glypiation, heme C attachment, hydroxylation, hypusine formation, iodination, isoprenylation, lipidation, lipoylation, malonylation, methylation, myristolylation, oxidation, palmitoylation, pegylation, phosphopantetheinylation, phosphorylation, prenylation, propionylation, retinylidene Schiff base formation, S-glutathionylation, S-nitrosylation, S-sulfenylation, selenation, succinylation, sulfination, ubiquitination, and C-terminal amidation. A post-translational modification includes modifications of the amino terminus and/or the carboxyl terminus of a peptide. Modifications of the terminal amino group include, but are not limited to, des-amino, N-lower alkyl, N-di-lower alkyl, and N-acyl modifications. Modifications of the terminal carboxy group include, but are not limited to, amide, lower alkyl amide, dialkyl amide, and lower alkyl ester modifications (e.g., wherein lower alkyl is C1-C4 alkyl). A post-translational modification also includes modifications, such as but not limited to those described above, of amino acids falling between the amino and carboxy termini. The term post-translational modification can also include peptide modifications that include one or more detectable labels.

As used herein, the term “binding agent” refers to a nucleic acid molecule, a peptide, a polypeptide, a protein, or a small molecule that id capable of specific binding to a binding target, e.g., amino acid residue of a peptide in complex with a coupler. The binding of a binding agent to the coupler-amino acid complex, or a group of coupler-amino acid complexes, refers to any covalent or non-covalent interaction between the binding agent and the coupler-amino acid complex. The term “group of coupler-amino acid complexes” refers to a set of amino acids that are bound by the same coupler-amino acid complex binding agent. A binding agent may also be a chimeric binding agent, composed of two or more types of molecules, such as a nucleic acid molecule-peptide chimeric binding agent. In some embodiments, binding agent comprises a polypeptide. In preferred embodiments, binding agent is a polypeptide. In some embodiments, binding agent is not a nucleic acid molecule. In some embodiments, binding agent is not a small molecule. A binding agent may be a naturally occurring, synthetically produced, or recombinantly expressed molecule. A binding agent may preferably bind to a modified or labeled amino acid (e.g., an amino acid that has been labeled by a chemical reagent and/or by a coupler) over a non-modified or unlabeled amino acid. In some embodiments, the binding agent binds to the coupler-amino acid complex, but does not essentially bind to the corresponding amino acid separated from the coupler-amino acid complex, or affinity of the binding agent for the corresponding amino acid separated from the coupler-amino acid complex is reduced compared to affinity of the binding agent for the coupler-amino acid complex by at least one order of magnitude. A binding agent may exhibit selective binding to one of the 20 possible natural amino acids in complex with the coupler, and bind with very low affinity or not at all to the other 19 natural amino acid residues in complex with the same molecule. A binding agent may also exhibit less selective binding, where the binding agent may bind with similar or different affinity to two or more different amino acid residues that are available in complex with the coupler, and thus, providing identification for these amino acid residues. This type of identification is still relevant for protein identification, since narrowing down the possibility of an amino acid at a certain position in the polypeptide sequence is still relevant for database searches. In one embodiment, binding agent may specifically bind to two or more structurally similar amino acid residues (such as small hydrophobic amino acids) in complex with the coupler, and the identity of the binding agent may be used to obtain identities of these amino acid residues, which then may be used for.

The terms “specific binding” and “specific recognition” are used interchangeably herein and generally refer to an engineered binding agent that binds to a particular amino acid residue in complex with the coupler more readily than it would bind to a random amino acid residue in complex with the coupler (there is a detectable relative increase in the binding of the binding agent to a specific or group of coupler-amino acid complexes). The term “specificity” is used herein to qualify the relative affinity by which an engineered binding agent binds to a cognate amino acid residue in complex with the coupler. Specific binding typically means that an engineered binding agent binds to a cognate amino acid residue in complex with the coupler at least twice more likely that to a random, non-cognate amino acid residue in complex with the coupler (a 2:1 ratio of specific to non-specific binding). Non-specific binding refers to background binding, and is the amount of signal that is produced in a binding assay between an engineered binding agent and a non-cognate amino acid residue in complex with the coupler immobilized on a solid support. In some embodiments, specific binding refers to binding between an engineered binding agent and a cognate amino acid residue in complex with the coupler with a dissociation constant (Kd) of 500 nM or less.

As used herein, the term “selective binding” refers to binding agents capable of binding specifically or preferentially to a particular amino acid in complex with the coupler (such as specific binding of a complex of a coupler and Trp, compared to other complexes of the coupler and a different amino acid), or capable of binding specifically or preferentially to a few or several structurally similar amino acids in complex with the coupler (such as specific binding of a complex of a coupler and Arg, or a complex of the coupler and Lys, compared to other complexes of the coupler with different amino acids). Examples of structurally similar amino acids include positively charged amino acids, negatively charged amino acids, small hydrophobic amino acids, aromatic amino acids. Preferential binding refers to binding with a greater affinity for a specific or subgroup of coupler-amino acid complexes compared to other specific or subgroup coupler-amino acid complexes.

As used herein, a polynucleotide or polypeptide variant, mutant, homologue, or modified version include polynucleotides or polypeptides that share nucleic acid or amino acid sequence identity with a reference polynucleotide or polypeptide. For example, variant or modified polypeptide generally exhibits about 25%, 30%, 40%, 50%, 60%, 70%, 80%, 85%, 86%, 87%, 88%, 89%, 90%, 91%, 92%, 93%, 94%, 95%, 96%, 97%, 98%, 99% or more sequence identity to a corresponding wild-type or unmodified polypeptide. The term “modified” or “engineered” (or “variant” or mutant”) as used in reference to polynucleotides and polypeptides implies that such molecules are created by human intervention and/or they are non-naturally occurring. A variant, mutant or modified polypeptide is not limited to any variant, mutant or modified polypeptide made or generated by a particular method of making and includes, for example, a variant, mutant or modified polypeptide made or generated by genetic selection, protein engineering, directed evolution, de novo recombinant DNA techniques, or combinations thereof. A mutant, variant or modified polypeptide is altered in primary amino acid sequence by substitution, addition, or deletion of amino acid residues. In some embodiments, variants of a polypeptide displaying only non-substantial or negligible differences in structure can be generated by making conservative amino acid substitutions in the modified polypeptide. By doing this, modified polypeptide variants that comprise a sequence having at least 90% (90%, 91%, 92%, 93%, 94%, 95%, 96%, 97%, 98%, and 99%) sequence identity with the modified polypeptide sequences can be generated, retaining at least one functional activity of the polypeptide. Examples of conservative amino acid changes are known in the art. Examples of non-conservative amino acid changes that are likely to cause major changes in protein structure are those, for example, that cause substitution of a hydrophilic residue to a hydrophobic residue. Methods of making targeted amino acid substitutions, deletions, truncations, and insertions are generally known in the art. For example, amino acid sequence variants can be prepared by mutations in the DNA. Methods for polynucleotide alterations are well known in the art, for example, Kunkel et al. (1987) Methods in Enzymol. 154:367-382; U.S. Pat. No. 4,873,192 and the references cited therein.

The term “sequence identity” is a measure of identity between proteins at the amino acid level and a measure of identity between nucleic acids at nucleotide level. The protein sequence identity may be determined by comparing the amino acid sequence in a given position in each sequence when the sequences are aligned. Similarly, the nucleic acid sequence identity may be determined by comparing the nucleotide sequence in a given position in each sequence when the sequences are aligned. “Sequence identity” means the percentage of identical subunits at corresponding positions in two sequences when the two sequences are aligned to maximize subunit matching, i.e., taking into account gaps and insertions. For example, the BLAST algorithm calculates percent sequence identity and performs a statistical analysis of the similarity between the two sequences.

The terms “corresponding to position(s)” or “position(s) . . . with reference to position(s)” of or within a polypeptide or a polynucleotide, such as recitation that nucleotides or amino acid positions “correspond to” nucleotides or amino acid positions of a disclosed sequence, such sequence set forth in the Sequence Listing, refers to nucleotides or amino acid positions identified in the polynucleotide or in the polypeptide upon alignment with the disclosed sequence using a standard alignment algorithm, such as the BLAST algorithm (NCBI). One skilled in the art can identify any given amino acid residue in a given polypeptide at a position corresponding to a particular position of a reference sequence, such as set forth in the Sequence Listing, by performing alignment of the polypeptide sequence with the reference sequence (for example, by using BLASTP publicly available through the NCBI website), matching the corresponding position of the reference sequence with the position in polypeptide sequence and thus identifying the amino acid residue within the polypeptide.

As used herein, the term “linker” refers to one or more of a nucleotide, a nucleotide analog, an amino acid, a peptide, a polypeptide, a polymer, or a non-nucleotide chemical moiety that is used to join two molecules. A linker may be used to join a binding agent with a coding tag, a recording tag with a polypeptide, a polypeptide with a support, a recording tag with a solid support, etc. In certain embodiments, a linker joins two molecules via enzymatic reaction or chemistry reaction (e.g., click chemistry).

The term “coupler” as used herein refers to any molecule or moiety that comprises a terminal amino acid reactive group, which reacts to and binds the terminal amino acid of a polypeptide. In certain embodiments, the terminal amino acid reactive group may comprise a primary amine reactive group that conjugates to the free amine at the N-terminus of the polypeptide. In other embodiments, the terminal amino acid reactive group may comprise a C-terminal reactive group that conjugates to a carboxylic group at the C-terminal end of the polypeptide. In some embodiments, the coupler may further comprise an amino acid cleaving group that initiates cleavage of the coupler-polypeptide complex from the polypeptide resulting in release of the coupler-terminal amino acid complex from the polypeptide. In other embodiments, the coupler does not comprise an amino acid cleaving group. In some embodiments, the cleavage and release of the coupler-terminal amino acid complex from the polypeptide is achieved enzymatically, with a help of a modified dipeptidyl peptidase enzyme that has been selected to cleave the coupler-terminal amino acid complex off the polypeptide. In some embodiments, the coupler may further comprise an tethering group for attaching the released coupler-terminal amino acid complex to the solid support. In other embodiments, the coupler does not comprise the tethering group, and releasable attachment of the coupler-terminal amino acid complex to the solid support occurs via formation of a tethering complex.

The term “ligand” as used herein refers to any molecule or moiety connected to the compounds described herein. “Ligand” may refer to one or more ligands attached to a compound. In some embodiments, the ligand is a pendant group or binding site (e.g., the site to which the binding agent binds).

As used herein, the term “proteome” can include the entire set of proteins, polypeptides, or peptides (including conjugates or complexes thereof) expressed by a genome, cell, tissue, or organism at a certain time, of any organism. In one aspect, it is the set of expressed proteins in a given type of cell or organism, at a given time, under defined conditions. As used herein, the term “proteome” include subsets of a proteome, including but not limited to a kinome; a secretome; a receptome (e.g., GPCRome); an immunoproteome; a nutriproteome; a proteome subset defined by a post-translational modification (e.g., phosphorylation, ubiquitination, methylation, acetylation, glycosylation, oxidation, lipidation, and/or nitrosylation), such as a phosphoproteome (e.g., phosphotyrosine-proteome, tyrosine-kinome, and tyrosine-phosphatome), a glycoproteome, etc.; a proteome subset associated with a tissue or organ, a developmental stage, or a physiological or pathological condition; a proteome subset associated a cellular process, such as cell cycle, differentiation (or de-differentiation), cell death, senescence, cell migration, transformation, or metastasis; or any combination thereof.

The terminal amino acid at one end of a peptide or polypeptide chain that has a free amino group is referred to herein as the “N-terminal amino acid” (NTAA). The terminal amino acid at the other end of the chain that has a free carboxyl group is referred to herein as the “C-terminal amino acid” (CTAA). The amino acids making up a peptide may be numbered in order, with the peptide being “n” amino acids in length. As used herein, NTAA is considered the n^{th} amino acid (also referred to herein as the “n NTAA”). Using this nomenclature, the next amino acid is the n-1 amino acid, then the n-2 amino acid, and so on down the length of the peptide from the N-terminal end to C-terminal end. In certain embodiments, an NTAA, CTAA, or both may be modified or labeled with a moiety or a chemical moiety.

As used herein, the term “macromolecule comprises a component” refers to a situation where the component is either a part of the macromolecule, or directly attached to the macromolecule by means of one or more covalent bond(s), which unite them into a single molecule. Instead, the term “macromolecule associated with a component” indicates that the component may or may not be directly attached to the macromolecule by means of one or more covalent bond(s), but instead can be associated, or co-localized, with the macromolecule by means of non-covalent interactions, or, alternatively, be associated indirectly through a solid support (for example, when the macromolecule is attached to the solid support, and the component is independently attached to the solid support in a proximity to the macromolecule. For example, the term “the polypeptide is associated with a first stabilizing component” encompasses various possible ways for association between the polypeptide and the first stabilizing component (either direct, covalent or non-covalent association, or indirect association, such as association via a linker or via another object, such as via solid support). The terms “attaching”, “joining” and “linking” are used interchangeably and refer to either covalent or non-covalent attachment.

As used herein, the term “releasably attached” refers to covalent or non-covalent attachment of a complex to a solid support, wherein such attachment can be reversed under mild conditions, such as conditions that do not impair or compromise structure or functional activity of components of the complex.

As used herein, the term “barcode” refers to a nucleic acid molecule of about 2 to about 30 bases (e.g., 2, 3, 4, 5, 6, 7, 8, 9, 10, 11, 12, 13, 14, 15, 16, 17, 18, 19, 20, 21, 22, 23, 24, 25, 26, 27, 28, 29 or 30 bases) providing a unique identifier tag or origin information for a polypeptide, a binding agent, a set of binding agents from a binding cycle, a sample polypeptides, a set of samples, polypeptides within a compartment (e.g., droplet, bead, or separated location), polypeptides within a set of compartments, a fraction of polypeptides, a set of polypeptide fractions, a spatial region or set of spatial regions, a library of polypeptides, or a library of binding agents. A barcode can be an artificial sequence or a naturally occurring sequence. In certain embodiments, each barcode within a population of barcodes is different. In other embodiments, a portion of barcodes in a population of barcodes is different, e.g., at least about 10%, 15%, 20%, 25%, 30%, 35%, 40%, 45%, 50%, 55%, 60%, 65%, 70%, 75%, 80%, 85%, 90%, 95%, 97%, or 99% of the barcodes in a population of barcodes is different. A population of barcodes may be randomly generated or non-randomly generated. In certain embodiments, a population of barcodes are error-correcting or error-tolerant barcodes. Barcodes can be used to computationally deconvolute the multiplexed sequencing data and identify sequence reads derived from an individual polypeptide, sample, library, etc. A barcode can also be used for deconvolution of a collection of polypeptides that have been distributed into small compartments for enhanced mapping. For example, rather than mapping a peptide back to the proteome, the peptide is mapped back to its originating protein molecule or protein complex.

As used herein, the term “coding tag” refers to a polynucleotide with any suitable length, e.g., a nucleic acid molecule of about 2 bases to about 100 bases, including any integer including 2 and 100 and in between, that comprises identifying information for its associated binding agent. A “coding tag” may also be made from a “sequenceable polymer” (see, e.g., Niu et al., 2013, Nat. Chem. 5:282-292; Roy et al., 2015, Nat. Commun. 6:7237; Lutz, 2015, Polypeptides 48:4759-4767; each of which are incorporated by reference in its entirety). A coding tag may comprise an encoder sequence, which is optionally flanked by one spacer on one side or optionally flanked by a spacer on each side. A coding tag may also be comprised of an optional UMI and/or an optional binding cycle-specific barcode. A coding tag may be single stranded or double stranded. A double stranded coding tag may comprise blunt ends, overhanging ends, or both. A coding tag may refer to the coding tag that is directly attached to a binding agent, to a complementary sequence hybridized to the coding tag directly attached to a binding agent (e.g., for double stranded coding tags), or to coding tag information present in an extended recording tag. In certain embodiments, a coding tag may further comprise a binding cycle specific spacer or barcode, a unique molecular identifier, a universal priming site, or any combination thereof.

As used herein, the term “spacer” (Sp) refers to a nucleic acid molecule of about 1 base to about 20 bases (e.g., 1, 2, 3, 4, 5, 6, 7, 8, 9, 10, 11, 12, 13, 14, 15, 16, 17, 18, 19, or 20 bases) in length that is present on a terminus of a recording tag or coding tag. In certain embodiments, a spacer sequence flanks an encoder sequence of a coding tag on one end or both ends. Following binding of a binding agent to a polypeptide, annealing between complementary spacer sequences on their associated coding tag and recording tag, respectively, allows transfer of binding information through a primer extension reaction or ligation to the recording tag, coding tag, or a di-tag construct. Sp′ refers to spacer sequence complementary to Sp. Preferably, spacer sequences within a library of binding agents possess the same number of bases. A common (shared or identical) spacer may be used in a library of binding agents. A spacer sequence may have a “cycle specific” sequence in order to track binding agents used in a particular binding cycle. The spacer sequence (Sp) can be constant across all binding cycles, be specific for a particular class of polypeptides, or be binding cycle number specific. Polypeptide class-specific spacers permit annealing of a cognate binding agent's coding tag information present in an extended recording tag from a completed binding/extension cycle to the coding tag of another binding agent recognizing the same class of polypeptides in a subsequent binding cycle via the class-specific spacers. Only the sequential binding of correct cognate pairs results in interacting spacer elements and effective primer extension. A spacer sequence may comprise sufficient number of bases to anneal to a complementary spacer sequence in a recording tag to initiate a primer extension (also referred to as polymerase extension) reaction, or provide a “splint” for a ligation reaction, or mediate a “sticky end” ligation reaction. A spacer sequence may comprise a fewer number of bases than the encoder sequence within a coding tag.

As used herein, the term “recording tag” refers to a moiety, e.g., a chemical coupling moiety, a nucleic acid molecule, or a sequenceable polymer molecule (see, e.g., Niu et al., 2013, Nat. Chem. 5:282-292; Roy et al., 2015, Nat. Commun. 6:7237; Lutz, 2015, Polypeptides 48:4759-4767; each of which are incorporated by reference in its entirety) to which identifying information of a coding tag can be transferred, or from which identifying information about the polypeptide (e.g., UMI information) associated with the recording tag can be transferred to the coding tag. Identifying information can comprise any information characterizing a molecule such as information pertaining to sample, fraction, partition, spatial location, interacting neighboring molecule(s), cycle number, etc. Additionally, the presence of UMI information can also be classified as identifying information. In certain embodiments, after a binding agent binds to a polypeptide, information from a coding tag linked to a binding agent can be transferred to the recording tag associated with the polypeptide while the binding agent is bound to the polypeptide. In other embodiments, after a binding agent binds to a polypeptide, information from a recording tag associated with the polypeptide can be transferred to the coding tag linked to the binding agent while the binding agent is bound to the polypeptide. A recording tag may be directly linked to a polypeptide, linked to a polypeptide via a multifunctional linker, or associated with a polypeptide by virtue of its proximity (or co-localization) on a support. A recording tag may be linked via its 5′ end or 3′ end or at an internal site, as long as the linkage is compatible with the method used to transfer coding tag information to the recording tag or vice versa. A recording tag may further comprise other functional components, e.g., a universal priming site, unique molecular identifier, a barcode (e.g., a sample barcode, a fraction barcode, spatial barcode, a compartment tag, etc.), a spacer sequence that is complementary to a spacer sequence of a coding tag, or any combination thereof. The spacer sequence of a recording tag is preferably at the 3′-end of the recording tag in embodiments where polymerase extension is used to transfer coding tag information to the recording tag.

As used herein, the term “primer extension”, also referred to as “polymerase extension”, refers to a reaction catalyzed by a nucleic acid polymerase (e.g., DNA polymerase) whereby a nucleic acid molecule (e.g., oligonucleotide primer, spacer sequence) that anneals to a complementary strand is extended by the polymerase, using the complementary strand as template.

As used herein, the term “unique molecular identifier” or “UMI” refers to a nucleic acid molecule of about 3 to about 40 bases (3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 bases) in length providing a unique identifier tag for each polypeptide, polypeptide or binding agent to which the UMI is linked. A polypeptide UMI can be used to computationally deconvolute sequencing data from a plurality of extended recording tags to identify extended recording tags that originated from an individual polypeptide. A polypeptide UMI can be used to accurately count originating polypeptide molecules by collapsing NGS reads to unique UMIs. A binding agent UMI can be used to identify each individual molecular binding agent that binds to a particular polypeptide. For example, a UMI can be used to identify the number of individual binding events for a binding agent specific for a single amino acid that occurs for a particular peptide molecule. It is understood that when UMI and barcode are both referenced in the context of a binding agent or polypeptide, that the barcode refers to identifying information other that the UMI for the individual binding agent or polypeptide (e.g., sample barcode, compartment barcode, binding cycle barcode).

As used herein, the term “universal priming site” or “universal primer” or “universal priming sequence” refers to a nucleic acid molecule, which may be used for library amplification and/or for sequencing reactions. A universal priming site may include, but is not limited to, a priming site (primer sequence) for PCR amplification, flow cell adaptor sequences that anneal to complementary oligonucleotides on flow cell surfaces enabling bridge amplification in some next generation sequencing platforms, a sequencing priming site, or a combination thereof. Universal priming sites can be used for other types of amplification, including those commonly used in conjunction with next generation digital sequencing. For example, extended recording tag molecules may be circularized and a universal priming site used for rolling circle amplification to form DNA nanoballs that can be used as sequencing templates (Drmanac et al., 2009, Science 327:78-81). Alternatively, recording tag molecules may be circularized and sequenced directly by polymerase extension from universal priming sites (Korlach et al., 2008, Proc. Natl. Acad. Sci. 105:1176-1181). The term “forward” when used in context with a “universal priming site” or “universal primer” may also be referred to as “5′” or “sense”. The term “reverse” when used in context with a “universal priming site” or “universal primer” may also be referred to as “3′” or “antisense”.

As used herein, the term “recording tag extended after information transfer” or “extended recording tag” refers to a recording tag to which information of at least one binding agent's coding tag (or its complementary sequence) has been transferred following binding of the binding agent to a polypeptide. Information of the coding tag may be transferred to the recording tag directly (e.g., ligation) or indirectly (e.g., primer extension). Information of a coding tag may be transferred to the recording tag enzymatically or chemically. An extended recording tag may comprise binding agent information of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 125, 150, 175, 200 or more coding tags. The base sequence of an extended recording tag may reflect the temporal and sequential order of binding of the binding agents identified by their coding tags, may reflect a partial sequential order of binding of the binding agents identified by the coding tags, or may not reflect any order of binding of the binding agents identified by the coding tags. In certain embodiments, the coding tag information present in the extended recording tag represents with at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity the polypeptide sequence being analyzed. In certain embodiments where the extended recording tag does not represent the polypeptide sequence being analyzed with 100% identity, errors may be due to off-target binding by a binding agent, or to a “missed” binding cycle (e.g., because a binding agent fails to bind to a polypeptide during a binding cycle, because of a failed primer extension reaction), or both.

As used herein, the term “solid support”, “solid surface”, or “solid substrate”, or “sequencing substrate”, or “substrate” refers to any solid material, including porous and non-porous materials, to which a polypeptide or a component of polypeptide, or molecules associated with a component of polypeptide can be associated directly or indirectly, by any means known in the art, including covalent and non-covalent interactions, or any combination thereof. A solid support may be two-dimensional (e.g., planar surface) or three-dimensional (e.g., gel matrix or bead). A solid support can be any support surface including, but not limited to, a bead, a microbead, an array, a glass surface, a silicon surface, a plastic surface, a filter, a membrane, a PTFE membrane, a PTFE membrane, a nitrocellulose membrane, a nitrocellulose-based polymer surface, nylon, a silicon wafer chip, a flow through chip, a flow cell, a biochip including signal transducing electronics, a channel, a microtiter well, an ELISA plate, a spinning interferometry disc, a nitrocellulose membrane, a nitrocellulose-based polymer surface, a polymer matrix, a nanoparticle, or a microsphere. Materials for a solid support include but are not limited to acrylamide, agarose, cellulose, dextran, nitrocellulose, glass, gold, quartz, polystyrene, polyethylene vinyl acetate, polypropylene, polyester, polymethacrylate, polyacrylate, polyethylene, polyethylene oxide, polysilicates, polycarbonates, poly vinyl alcohol (PVA), Teflon, fluorocarbons, nylon, silicon rubber, polyanhydrides, polyglycolic acid, polyvinylchloride, polylactic acid, polyorthoesters, functionalized silane, polypropylfumerate, collagen, glycosaminoglycans, polyamino acids, dextran, or any combination thereof. Solid supports further include thin film, membrane, bottles, dishes, fibers, woven fibers, shaped polymers such as tubes, particles, beads, microspheres, microparticles, or any combination thereof. For example, when solid surface is a bead, the bead can include, but is not limited to, a ceramic bead, a polystyrene bead, a polymer bead, a polyacrylate bead, a methylstyrene bead, an agarose bead, a cellulose bead, a dextran bead, an acrylamide bead, a solid core bead, a porous bead, a paramagnetic bead, a glass bead, a controlled pore bead, a silica-based bead, or any combinations thereof. A bead may be spherical or an irregularly shaped. A bead or support may be porous. A bead's size may range from nanometers, e.g., 100 nm, to millimeters, e.g., 1 mm. In certain embodiments, beads range in size from about 0.2 micron to about 200 microns, or from about 0.5 micron to about 5 micron. In some embodiments, beads can be about 1, 1.5, 2, 2.5, 2.8, 3, 3.5, 4, 4.5, 5, 5.5, 6, 6.5, 7, 7.5, 8, 8.5, 9, 9.5, 10, 10.5, 15, or 20 μm in diameter. In certain embodiments, “a bead” solid support may refer to an individual bead or a plurality of beads. In some embodiments, the solid surface is a nanoparticle. In certain embodiments, the nanoparticles range in size from about 1 nm to about 500 nm in diameter, for example, between about 1 nm and about 20 nm, between about 1 nm and about 50 nm, between about 1 nm and about 100 nm, between about 10 nm and about 50 nm, between about 10 nm and about 200 nm, between about 50 nm and about 100 nm, between about 50 nm and about 150, between about 50 nm and about 200 nm, between about 100 nm and about 200 nm, or between about 200 nm and about 500 nm in diameter. In some embodiments, the nanoparticles can be about 10 nm, about 50 nm, about 100 nm, about 150 nm, about 200 nm, about 300 nm, or about 500 nm in diameter. In some embodiments, the nanoparticles are less than about 200 nm in diameter. In some embodiments, the solid support is covered with a coating or functionalized. In some embodiments, the solid support is resistant to the basic and acidic pH, chemicals and buffers used for Edman degradation. In some embodiments, the solid support is resistant to the chemical reactions and conditions used for cleavage of the peptide bond between the terminal amino acid and the penultimate terminal amino acid of the polypeptide within the coupler-polypeptide complex. In some embodiments, the coating provides attachment points for affixing or tethering the components of the claimed methods, such as polypeptides, coupler, stabilizing components, to the solid support. In some embodiments, the solid support or the coating of the solid support is resistant to non-specific adherence of polypeptides or other reaction components, so as to minimize background signals.

As used herein, the term “associated” or “linked” refers to a non-random spatial proximity or co-localization of two or more molecules due to a direct or an indirect binding. In some embodiments, associated or linked molecules are directly attached to each other. In some embodiments, associated or linked molecules are attached to each other via a linker or via a linking agent. In some embodiments, attachment may occur via formation of covalent bonds. In some embodiments, attachment may occur via formation of non-covalent interactions, such as van der Walls interactions, hydrogen bonding, and/or electrostatic interactions (ionic bonding). As an example, when a polypeptide, an associated recording tag and an associated first stabilizing component attached to a solid support are provided, each molecule (polypeptide, recording tag and first stabilizing component) may be directly attached to the solid support independent of the other molecules. Moreover, in some embodiments, none of these molecules may be directly attached to the solid support, and all of them are attached to the solid support via a linker. In preferred embodiments, the linker forms covalent bonds with the solid support and with at least one of these molecules. In some embodiments, all three molecules are connected to each other by covalent bonds. As used herein, the term “releasably attached” refers to a method of attachment of two or more substances, where these substances can be attached and dis-attached to each other in a controlled manner, without affecting functional activities of the substances. Releasable attachment may include formation of covalent bonds, or may include formation of non-covalent bonds. Examples of releasable attachment with covalent bonds include attachment through formation of S—S bonds that can be disrupted in a controlled manner via addition of a reducing agent. Examples of releasable attachment with non-covalent bonds include attachment through DNA hybridization that can be disrupted in a controlled manner via heat or addition of denaturing reagents such as NaOH or formamide.

The term “detectable label” as used herein refers to a substance which can indicate the presence of another substance when associated with it. The detectable label can be a substance that is linked to or incorporated into the substance to be detected. In some embodiments, a detectable label is suitable for allowing for detection and also quantification, for example, a detectable label that emitting a detectable and measurable signal. Examples of detectable labels include a dye, a fluorophore, a chromophore, a fluorescent nanoparticle (e.g. quantum dot), a radiolabel, an enzyme (e.g. alkaline phosphatase, luciferase or horseradish peroxidase), or a chemiluminescent or bioluminescent molecule.

As used herein, the term “nucleic acid molecule” or “polynucleotide” refers to a single- or double-stranded polynucleotide containing deoxyribonucleotides or ribonucleotides that are linked by 3′-5′ phosphodiester bonds, as well as polynucleotide analogs. A nucleic acid molecule includes, but is not limited to, DNA, RNA, and cDNA. A polynucleotide analog may possess a backbone other than a standard phosphodiester linkage found in natural polynucleotides and, optionally, a modified sugar moiety or moieties other than ribose or deoxyribose. Polynucleotide analogs contain bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide. Examples of polynucleotide analogs include, but are not limited to xeno nucleic acid (XNA), bridged nucleic acid (BNA), glycol nucleic acid (GNA), peptide nucleic acids (PNAs), γPNAs, morpholino polynucleotides, locked nucleic acids (LNAs), threose nucleic acid (TNA), 2′-O-Methyl polynucleotides, 2′-O-alkyl ribosyl substituted polynucleotides, phosphorothioate polynucleotides, and boronophosphate polynucleotides. A polynucleotide analog may possess purine or pyrimidine analogs, including for example, 7-deaza purine analogs, 8-halopurine analogs, 5-halopyrimidine analogs, or universal base analogs that can pair with any base, including hypoxanthine, nitroazoles, isocarbostyril analogues, azole carboxamides, and aromatic triazole analogues, or base analogs with additional functionality, such as a biotin moiety for affinity binding. In some embodiments, the nucleic acid molecule or oligonucleotide is a modified oligonucleotide. In some embodiments, the nucleic acid molecule or oligonucleotide is a DNA with pseudo-complementary bases, a DNA with protected bases, an RNA molecule, a BNA molecule, an XNA molecule, a LNA molecule, a PNA molecule, a γPNA molecule, or a morpholino DNA, or a combination thereof. In some embodiments, the nucleic acid molecule or oligonucleotide is backbone modified, sugar modified, or nucleobase modified. In some embodiments, the nucleic acid molecule or oligonucleotide has nucleobase protecting groups such as Alloc, electrophilic protecting groups such as thiranes, acetyl protecting groups, nitrobenzyl protecting groups, sulfonate protecting groups, or traditional base-labile protecting groups.

As used herein, “nucleic acid sequencing” means the determination of the identity and order of at least a portion of nucleotides in the nucleic acid molecule or in a sample of nucleic acid molecules. Similarly, “polypeptide sequencing” means the determination of the identity and order of at least a portion of amino acids in the polypeptide molecule or in a sample of polypeptide molecules.

As used herein, “next generation sequencing” refers to high-throughput sequencing methods that allow the sequencing of millions to billions of molecules in parallel. Examples of next generation sequencing methods include sequencing by synthesis, sequencing by ligation, sequencing by hybridization, polony sequencing, ion semiconductor sequencing, and pyrosequencing. By attaching primers to a solid substrate and a complementary sequence to a nucleic acid molecule, a nucleic acid molecule can be hybridized to the solid substrate via the primer and then multiple copies can be generated in a discrete area on the solid substrate by using polymerase to amplify (these groupings are sometimes referred to as polymerase colonies or polonies). Consequently, during the sequencing process, a nucleotide at a particular position can be sequenced multiple times (e.g., hundreds or thousands of times)—this depth of coverage is referred to as “deep sequencing.” Examples of high throughput nucleic acid sequencing technology include platforms provided by Illumina, BGI, Qiagen, Thermo-Fisher, and Roche, including formats such as parallel bead arrays, sequencing by synthesis, sequencing by ligation, capillary electrophoresis, electronic microchips, “biochips,” microarrays, parallel microchips, and single-molecule arrays (See e.g., Service, Science (2006) 311:1544-1546).

As used herein, “single molecule sequencing” or “third generation sequencing” refers to next-generation sequencing methods wherein reads from single molecule sequencing instruments are generated by sequencing of a single molecule of DNA. Unlike next generation sequencing methods that rely on amplification to clone many DNA molecules in parallel for sequencing in a phased approach, single molecule sequencing interrogates single molecules of DNA and does not require amplification or synchronization. Single molecule sequencing includes methods that need to pause the sequencing reaction after each base incorporation (‘wash-and-scan’ cycle) and methods which do not need to halt between read steps. Examples of single molecule sequencing methods include single molecule real-time sequencing (Pacific Biosciences), nanopore-based sequencing (Oxford Nanopore), duplex interrupted nanopore sequencing, and direct imaging of DNA using advanced microscopy.

As used herein, “analyzing” the polypeptide or the polynucleotide means to identify, detect, quantify, characterize, distinguish, or a combination thereof, all or a portion of the components of the polypeptide or the polynucleotide. For example, analyzing a polypeptide includes determining all or a portion of the amino acid sequence (contiguous or non-continuous) of the peptide; analyzing a polynucleotide includes determining all or a portion of the nucleotide sequence (contiguous or non-continuous) of the polynucleotide. Analyzing a polypeptide also includes partial identification of a component of the polypeptide. For example, partial identification of amino acids in the polypeptide protein sequence can identify an amino acid in the protein as belonging to a subset of possible amino acids. Analysis typically begins with analysis of the n NTAA, and then proceeds to the next amino acid of the peptide (i.e., n-1, n-2, n-3, and so forth). This is accomplished by elimination of the n NTAA, thereby converting the n-1 amino acid of the peptide to an N-terminal amino acid (referred to herein as the “n-1 NTAA”). Analyzing the peptide may also include determining the presence and frequency of post-translational modifications on the peptide, which may or may not include information regarding the sequential order of the post-translational modifications on the peptide. Analyzing the peptide may also include determining the presence and frequency of epitopes in the peptide, which may or may not include information regarding the sequential order or location of the epitopes within the peptide. Analyzing the peptide may include combining different types of analysis, for example obtaining epitope information, amino acid sequence information, post-translational modification information, or any combination thereof.

As used herein, the term “cleavase” refers to any exopeptidase that has been modified from an unmodified or wild-type exopeptidase to cleave a single modified amino acid from the N-terminus of a peptide. Cleavase enzyme may be derived from an unmodified or wild-type dipeptide dipeptidyl aminopeptidase. As compared to an unmodified or wild-type dipeptidyl aminopeptidase which removes the P1-P2 terminal amino acids from a peptide as a dipeptide at a time, a cleavase derived from a dipeptidyl aminopeptidase removes a labeled P1 terminal amino acid from the peptide at a time. Examples of cleavases are disclosed in US published application 2021/0214701 A1, incorporated herein.

It is understood that aspects and embodiments of the invention described herein include “consisting of” and/or “consisting essentially of” aspects and embodiments.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Other objects, advantages and features of the present invention will become apparent from the following specification taken in conjunction with the accompanying drawings.

Provided herein are methods and kits for performing identification of a terminal amino acid of a polypeptide, or a polypeptide sequencing reaction. One exemplary embodiment provides a method for identifying a terminal amino acid of a polypeptide, comprising: providing a polypeptide and an associated recording tag attached to a solid support; contacting the polypeptide with a coupler, wherein the coupler binds to a terminal amino acid of the polypeptide to form a coupler-polypeptide complex; attaching the coupler (as a part of coupler-polypeptide complex) to the solid support; cleaving the coupler-polypeptide complex from the polypeptide, thereby providing a coupler-amino acid complex attached to the solid support; contacting the coupler-amino acid complex with a binding agent capable of binding to the coupler-amino acid complex, wherein the binding agent comprises a coding tag with identifying information regarding the binding agent; transferring the information of the coding tag of the binding agent to the recording tag; analyzing the recording tag extended after information transfer, thereby identifying the terminal amino acid of the polypeptide.

In yet another embodiment, a method for identifying a polypeptide is provided, comprising: (a) providing a polypeptide and an associated recording tag attached to a solid support;

(b) contacting the polypeptide with a coupler, wherein the coupler binds to a terminal amino acid of the polypeptide to form a coupler-polypeptide complex;
(c) attaching the coupler to the solid support;
(d) cleaving the peptide bond between the terminal amino acid and the penultimate terminal amino acid of the polypeptide within the coupler-polypeptide complex, thereby exposing a new terminal amino acid of the polypeptide and generating a coupler-amino acid complex attached to the solid support;
(e) contacting the coupler-amino acid complex attached to the solid support with a binding agent capable of specific binding to the coupler-amino acid complex, wherein the binding agent comprises a coding tag with identifying information regarding the binding agent;
(f) following binding of the binding agent to the coupler-amino acid complex, transferring the information of the coding tag of the binding agent to the recording tag to generate an extended recording tag;
(g) releasing the coupler-amino acid complex from the solid support;
(h) repeating steps (b) through (g) at least one more time; and
(i) analyzing the extended recording tag after information transfer, thereby identifying a sequence of the polypeptide.

In yet another embodiment, a method for identifying a terminal amino acid of a polypeptide is provided, comprising: (a) providing a polypeptide, an associated recording tag and an associated first stabilizing component attached to a solid support; (b) contacting the polypeptide with a coupler, wherein the coupler binds to a terminal amino acid of the polypeptide to form a coupler-polypeptide complex, and wherein the coupler is linked to a second stabilizing component; (c) after binding of the coupler to the terminal amino acid of the polypeptide, releasably linking the first and second stabilizing components together to form a tethering complex between the first stabilizing component attached to the solid support and the second stabilizing component linked to the coupler-polypeptide complex; (d) cleaving the coupler-polypeptide complex from the polypeptide, thereby providing a coupler-amino acid complex releasably attached to the solid support via the tethering complex; (f) contacting the coupler-amino acid complex with a binding agent capable of specific binding to the coupler-amino acid complex, wherein the binding agent comprises a coding tag with identifying information regarding the binding agent; (g) transferring the information of the coding tag of the binding agent to the recording tag; (h) analyzing the recording tag extended after information transfer, thereby identifying the terminal amino acid of the polypeptide.

The methods disclosed herein are designed for high throughput identification of polypeptides, and at least 200, at least 500, at least 1000, at least 2000, at least 5000, or at least 1000 different polypeptides can be analyzed in parallel (in a single assay).

In some embodiments, methods disclosed herein further comprise attaching the polypeptide analyte to the recording tag optionally joined to the solid support before performing step (a).

In some embodiments of the disclosed methods, the binding agent comprises a polypeptide or an aptamer.

In some embodiments, the polypeptide analyte is associated with a first stabilizing component, and is further associated with a recording tag. These elements (first stabilizing component and recording tag) have different functions in the claimed method. The purpose of the first stabilizing component is to form a tethering complex with a second stabilizing component (attached to the coupler), and thus attach (temporarily) the coupler-amino acid complex to the solid support (indirectly via tethering complex) after cleaving the peptide bond between the terminal amino acid and the penultimate terminal amino acid of the polypeptide within the coupler-polypeptide complex. Instead, the purpose of the recording tag is usually to provide a means for transfer of information regarding the binding agent that binds to the coupler-amino acid complex from a coding tag associated with the binding agent to the recording tag. After the transfer of information, the recording tag is extended, since information encoded in the coding tag is transferred to the recording tag, for example, via polynucleotide extension or ligation. Different variants for association between the polypeptide, the first stabilizing component and recording tag are possible and disclosed herein. The first stabilizing component and recording tag can be independently attached to the polypeptide, or can be associated with the polypeptide by various ways disclosed herein (can be associated either directly or indirectly). In preferred embodiments, a particular way of association between the polypeptide, the first stabilizing component and recording tag is not important, as long as the first stabilizing component and recording tag remain functional (can be used to form a complex with the second stabilizing component, and can be used for information transfer, respectively). In addition, the polypeptide can be attached to the solid support by various ways (for example, directly, through a linker, through the recording tag, through the stabilizing component, through covalent and/or non-covalent interactions, or any combination thereof). Similarly, the coupler can be attached to a second stabilizing component by means of covalent and/or non-covalent interactions; and the binding agent can be attached to a coding tag by means of covalent and/or non-covalent interactions. In preferred embodiments, the coupler is attached to the second stabilizing component by covalent interactions, and the binding agent is attached to the coding tag by covalent interactions.

In preferred embodiments of the disclosed methods, the binding of the binding agent to the polypeptide analyte does not depend on the presence of the first stabilizing component and the second stabilizing component.

In different embodiments of the invention, after binding to the terminal amino acid of the polypeptide, the coupler can be either directly attached to the solid support (by a tethering group within the coupler), or can be indirectly attached to the solid support via formation of the tethering complex between the first and second stabilizing components. The latter method provides more control over the attachment reaction, since the formation of the tethering complex can be achieved by addition of a linking agent. The strength and reversibility of the binding of the coupler-amino acid complex to the solid support can be controlled by binding affinities of the stabilizing components to each other or to the linking agent. For example, when the first stabilizing component has a lower affinity to the linking agent in comparison to an affinity of the second stabilizing component to the linking agent, this may allow for efficient dissociation of the tethering complex after the information transfer. In addition, attachment of the coupler-amino acid complex through the tethering complex increases efficiency of information transfer after the binding agent binds to the isolated coupler-amino acid complex by ensuring sufficient spatial proximity between the coding tag and the recording tag. When the coupler-amino acid complex attaches to the solid support directly, the location of the attachment site may vary, and this creates suboptimal conditions for the following information transfer. The described attachment approach operates by transiently attaching the coupler-amino acid complex to the solid support after first and second stabilizing components form a tethering complex. Several kinds of stabilizing components can be employed, but in a preferred embodiment, the methods rely on a rapid means of reversibly attaching the coupler-amino acid complex to the solid support after it gets cleaved from the polypeptide.

In yet another embodiment, a method for identifying at least a portion of a sequence of a polypeptide is provided, comprising: (a) providing a polypeptide, an associated recording tag and an associated first stabilizing component attached to a solid support; (b) contacting the polypeptide with a coupler, wherein the coupler binds to a terminal amino acid of the polypeptide to form a coupler-polypeptide complex, and wherein the coupler comprises or is associated with a second stabilizing component; (c) after binding of the coupler to the terminal amino acid of the polypeptide, releasably linking the first and second stabilizing components together to form a tethering complex between the first stabilizing component attached to the solid support and the second stabilizing component linked to the coupler-polypeptide complex; (d) cleaving the coupler-polypeptide complex from the polypeptide, thereby exposing a new terminal amino acid of the polypeptide and providing a coupler-amino acid complex releasably attached to the solid support via the tethering complex; (e) contacting the coupler-amino acid complex with a binding agent capable of binding to the coupler-amino acid complex, wherein the binding agent comprises a coding tag with identifying information regarding the binding agent; (f) transferring the information of the coding tag of the binding agent to the recording tag; (g) releasing the coupler-amino acid complex from the solid support by breaking the tethering complex; (h) repeating steps (b) through (g) at least one more time; (i) analyzing the recording tag extended after information transfer, thereby identifying at least a portion of the sequence of the polypeptide.

In some embodiments, the terminal amino acid of the polypeptide is modified before contacting the polypeptide with the coupler to produce a modified terminal amino acid; the coupler binds to the modified terminal amino acid; a coupler-modified amino acid complex is provided after cleavage attached to the solid support; and the binding agent is capable of binding to the coupler-modified amino acid complex.

In some embodiments, the coupler-polypeptide complex is releasably attached to the solid support.

In some embodiments, the stabilizing components are linked upon introduction of a linking agent, the linking agent comprising a chemical reagent, a non-biological reagent, a biological reagent, or a combination thereof.

In some embodiments, the linking agent comprises a polypeptide.

In some embodiments, the stabilizing components are linked upon introduction of a linking agent, and no covalent bonds are formed during formation of the tethering complex.

In some embodiments, the coupler comprises a polynucleotide. In other embodiments, the coupler does not comprise a polynucleotide.

In some embodiments, the linking agent comprises a metal ion.

In some embodiments, the stabilizing components are linked upon exposure to light (or upon introduction of light). In some embodiments, the stabilizing components each comprises a polynucleotide. The linking agent may comprise at least one polynucleotide or nucleic acid comprising a sequence which hybridizes to at least one of the stabilizing components. In some embodiments, the light or linking agent induces uncaging of one or both of the stabilizing components, deblocking of one or both of the stabilizing components, isomerization of the stabilizing components, hybridization of the stabilizing components, and/or binding of the stabilizing components.

In some embodiments, the support is a polystyrene bead, a polyacrylate bead, a polymer bead, an agarose bead, a cellulose bead, a dextran bead, an acrylamide bead, a solid core bead, a porous bead, a paramagnetic bead, a glass bead, a controlled pore bead, a silica-based bead, or a combination thereof.

In some embodiments, the coding tag or the recording tag is a DNA molecule, an RNA molecule, a PNA molecule, a BNA molecule, an XNA, molecule, an LNA molecule, a γPNA molecule, or a combination thereof.

In some embodiments, the first stabilizing component is the same as the second stabilizing component. In other embodiments, the first stabilizing component is different from the second stabilizing component. In some embodiments, the stabilizing components are linked upon introduction of a linking agent, and the first stabilizing component has a lower affinity to the linking agent in comparison to an affinity of the second stabilizing component to the linking agent.

In some embodiments, the binding agent binds to the coupler-amino acid complex, but does not bind to the corresponding amino acid separated from the coupler-amino acid complex, or affinity of the binding agent for the corresponding amino acid separated from the coupler-amino acid complex is reduced compared to affinity of the binding agent for the coupler-amino acid complex by at least an order of magnitude.

In some embodiments, the binding agent comprises a plurality of binding agents, wherein each binding agent from the plurality of binding agents comprises a coding tag with identifying information regarding the binding agent, and at least one binding agent from the plurality of binding agents is capable of binding to the coupler-amino acid complex.

In some embodiments, the coupler is capable of binding to a N-terminal amino acid (NTAA) of the polypeptide or to a modified NTAA of the polypeptide. In some embodiments, the coupler binds to a post-translationally modified terminal amino acid.

In some embodiments, the recording tag extended after information transfer is analyzed using a nucleic acid sequencing method. In some embodiments, the nucleic acid sequencing method is sequencing by synthesis, sequencing by ligation, sequencing by hybridization, polony sequencing, ion semiconductor sequencing, or pyrosequencing. In some other embodiments, the nucleic acid sequencing method is single molecule real-time sequencing, nanopore-based sequencing, or direct imaging of DNA using advanced microscopy.

In some embodiments, transferring the information comprises contacting the coding tag with a reagent for transferring the information, the reagent comprising a reagent for primer extension reaction, a chemical ligation reagent or a biological ligation reagent.

In some embodiments, the coupler binds to a N-terminal amino acid (NTAA) of the polypeptide.

In some embodiments, prior to contacting the polypeptide with the coupler, the method further comprises the step of contacting the polypeptide with a N-terminal modifier agent to form the modified NTAA of the polypeptide.

In some embodiments, the method further comprises the step of releasing the coupler-amino acid complex or the coupler-modified amino acid complex from the solid support after transferring the information of the coding tag of the binding agent to the recording tag.

In some embodiments, the method further comprises the step of disrupting the tethering complex after the transfer of the information from the coding tag of the binding agent to the recording tag.

In some embodiments, releasing the coupler-amino acid complex or disrupting the tethering complex is conducted by introducing a destabilizing agent. In some embodiments, the disrupting is conducted by introducing a destabilizing agent, wherein the destabilizing agent comprises heat, a denaturing agent, an enzyme, or a competitor molecule.

In some embodiments, the method further comprises a washing step to remove the released coupler-amino acid complex.

In some embodiments, the polypeptide is obtained by fragmenting proteins from a biological sample. In some embodiments, the fragmenting is performed by contacting the proteins with a protease.

In some embodiments, the binding agent capable of binding to the coupler-amino acid complex is configured to bind specifically to the amino acid from the coupler-amino acid complex.

In some embodiments, the coupler-amino acid complex is cleaved from the polypeptide by a modified dipeptidyl peptidase enzyme. In some other embodiments, the coupler-amino acid complex is cleaved from the polypeptide by mild Edman degradation, Edmanase enzyme, or by applying acidic agents, such as applying anhydrous TFA.

In some embodiments, the linking of the stabilizing components forms a complex adequately or sufficiently stable for information transfer to occur from the coding tag to the recording tag.

In some embodiments, transferring the identifying information of the coding tag to the recording tag is effected by primer extension. In other embodiments, transferring the identifying information of the coding tag to the recording tag is effected by ligation.

In some embodiments, the coupler is configured to bind to a C-terminal amino acid residue of the polypeptide.

In some embodiments, the coding tag and/or the recording tag comprises: a universal priming site comprising a priming site for amplification, sequencing, or both; a unique molecule identifier (UMI); a barcode; a spacer at its 3′-terminus; and/or a 3′ blocking group. In some other embodiments, the coding tag further comprises a spacer, a binding cycle specific barcode, a unique molecular identifier, a universal priming site, a terminator nucleotide, or any combination thereof.

In some embodiments, the plurality of polypeptides to be analyzed or sequenced are spaced apart on the solid support at an average distance >50 nm.

In some embodiments, transferring the information of the coding tag to the recording tag is mediated by a DNA ligase, by a DNA polymerase, or by chemical ligation.

In some embodiments, the binding agent and the coding tag are joined by a linker.

In some embodiments, the binding agent is an aminopeptidase or variant, mutant, or modified protein thereof; an aminoacyl tRNA synthetase or variant, mutant, or modified protein thereof; an anticalin or variant, mutant, or modified protein thereof; a ClpS, ClpS2, or variant, mutant, or modified protein thereof; a UBR box protein or variant, mutant, or modified protein thereof; or an antibody or antigen-recognizing fragment thereof; a metalloenzyme or variant, mutant, or modified protein thereof; an amino acid binding periplasmic binding protein or variant, mutant, or modified protein thereof.

In yet another embodiment, a kit for analyzing a polypeptide is provided, comprising: a coupler, wherein the coupler is configured to bind to a terminal amino acid of the polypeptide to form a coupler-polypeptide complex; a reagent for cleaving the coupler-polypeptide complex from the polypeptide and forming a coupler-amino acid complex; a binding agent capable of binding to the coupler-amino acid complex.

In some embodiments, the kit further comprises a coding tag with identifying information regarding the binding agent and/or a recording tag configured to be associated with the polypeptide. In some embodiments, the coding tag or the recording tag comprises a unique molecular identifier (UMI) or a barcode sequence.

In some embodiments, the kit further comprises a solid support, wherein the polypeptide or an associated recording tag is configured to be attached to the solid support either directly or via a linker.

In some embodiments, the binding agent capable of binding to the coupler-amino acid complex is configured to bind specifically to the amino acid from the coupler-amino acid complex.

In some embodiments, the coupler-polypeptide complex is configured to be releasably attached to the solid support.

In some embodiments, the kit further comprises a terminal modifier agent configured to modify the terminal amino acid of the polypeptide and produce a modified terminal amino acid of the polypeptide, wherein the coupler is configured to bind to the modified terminal amino acid of the polypeptide.

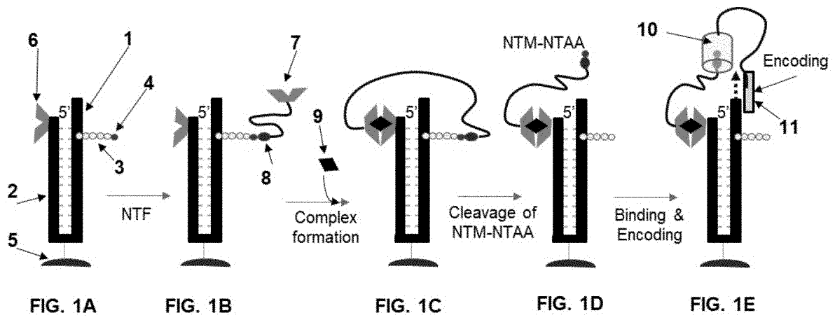

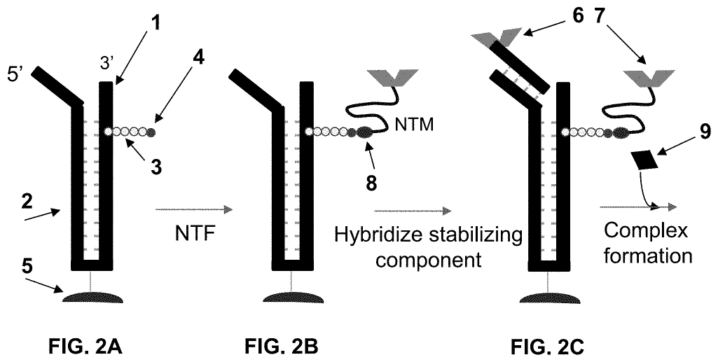

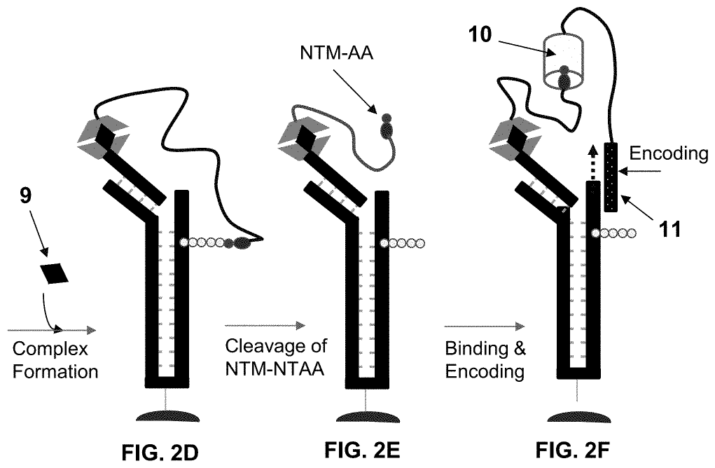

In some embodiments, the provided methods for performing a tethering reaction are performed in an assay for sequencing or analysis of the polypeptides. Before or after performing a tethering reaction (see e.g., FIGS. 1A-1B and FIGS. 2A-2C), other steps of an assay for analysis of the polypeptide may be performed. In some embodiments, the provided methods for performing a tethering reaction are compatible with a further information transfer step, such as information transfer between nucleic acids associated with the binding agent and the polypeptide. In some embodiments, the information transfer is between a nucleic acid tag associated with the binding agent and a nucleic acid tag associated with the polypeptide (e.g., by extension or ligation).

In some embodiments, the provided methods for performing a tethering reaction are performed without use of stabilizing components. In these embodiments, after contacting the immobilized polypeptide, the coupler is attached to the solid support via a tethering moiety or via a linker. In some embodiments, the tethering moiety that reacts with a moiety on the solid support is a part of the coupler. In other embodiments, the coupler does not comprise a tethering moiety, but instead comprises a reactive handle, and the tethering moiety that reacts with a moiety on the solid support comprises a complementary reactive handle that reacts specifically with the reactive handle on the coupler. In some embodiments, the tethering moiety that can be used for attaching the coupler to the solid support comprises one of the following moieties: a click chemistry moiety, an aldehyde, an azide, an alkyne, a maleimide, a thiol, an epoxide, a nucleophile, an inverse electron demand Diels-Alder (iEDDA) group, a moiety for a Staudinger reaction, dibenzocyclooctyne (DBCO), tetrazine, succinimide, allyl, vinyl, or a derivative thereof.

In some embodiments, the solid support is a functionalized solid support that comprises functional groups that react with one or more components of the claimed methods, such as polypeptide, recording tag, capture DNA, coupler, stabilizing components. In some embodiments, the coupler can be tethered to a functionalized solid support directly via a tethering moiety, or via a linker. For example, if the functionalized solid support comprises azide groups, then the coupler comprising an alkyne that reacts with the azide can be tethered directly to the support. In preferred embodiments, the groups used for tethering the coupler to the functionalized solid support form a bioorthogonal reactive pair, namely they would not react with other components of the system or with other biomolecules.

Forming a Tethering Complex with Linked Stabilizing Components

In some embodiments of the disclosed methods, a first stabilizing component may be directly or indirectly associated with or joined to the polypeptide analyte. In these embodiments, a second stabilizing component may be directly or indirectly associated with or joined to the coupler. In these embodiments, each of the stabilizing components is attached to or associated with the coupler and the polypeptide, respectively, at a site different from the binding site between the coupler and the polypeptide.

A particular way of association between the polypeptide analyte and the first stabilizing component (as well as between the binding agent and the second stabilizing component) is not limited. The purpose of using the stabilizing components is to stabilize or temporarily stabilize interaction between the polypeptide analyte and the binding agent that binds to the polypeptide analyte. To stabilize binding, a stabilizing component may be directly or indirectly associated with or joined to the polypeptide analyte. In some embodiments, this stabilization is achieved by introducing a linking agent that binds to the first stabilizing component and to the second stabilizing component and forms a stable complex that keeps the binding agent and the polypeptide analyte in a close proximity. This would allow for a low affinity binding agent with a relatively high dissociation constant (Kd) to interact with the polypeptide analyte for an extended period of time. In these embodiments, the stabilizing components should only be located within a close proximity from the polypeptide-binding agent complex in order to bind to the linking agent and stabilize the complex. However, the way(s) by which the stabilizing components are associated with their corresponding partners are be limited.

The described stabilization approach operates by transiently “cross-linking” the binding agent and the polypeptide analyte on a support after binding event forming a stable complex. Several kinds of stabilizing components can be employed, but in a preferred embodiment the stabilization methods rely on a rapid means of reversibly coupling the DNA-polypeptide complex to the binding agent after it binds to the polypeptide analyte.

In some embodiments, once the stabilizing components are linked, the binding agent and the polypeptide analyte remain bound. In some embodiments, once the stabilizing components are linked, the binding agent and the polypeptide analyte are released from each other, remaining in the vicinity of each other by virtue of the linked stabilizing components. In either case, when the stabilizing components remain linked, the binding agent remains either bound or in proximity to the polypeptide analyte, so that the process of information transfer (encoding) can occur (the identifying information regarding the binding agent is transferred from the coding tag to the recording tag).

In some embodiments, the linking agent contain a structure that allows the linking agent to bind to the first and second stabilizing components to form a stable complex comprising the binding agent, the polypeptide analyte and the stabilizing components. So long the linking agent and stabilizing components contain structures that allow binding between the linking agent and stabilizing components to form the stable complex, the types of the structures and binding between the linking agent and stabilizing components are not be limited to any specific types of the structures and binding. Accordingly, any suitable structures of stabilizing components can be used in the claimed methods as long as the stabilizing components can bind to linking agent and form a stable complex that stabilizes binding between the binding agent and the macromolecule. Exemplary structures of the linking agent, the first and second stabilizing components are disclosed in U.S. Ser. No. 11/169,157 B2, incorporated herein.

In some embodiments, the stabilizing components and linking agents with the desired binding affinity can be selected and used for the methods provided herein for the binding reaction. In some embodiments, the relative affinity of stabilizing components to each other and/or to the linking agent is at least as high as the affinity of the binding agent to the polypeptide analyte.

Provided herein are also methods for performing a tethering reaction that forms a tethering complex. To form a tethering complex, a coupler is covalently or non-covalently associated with a terminal amino acid of a polypeptide, and the coupler and the polypeptide each comprises, is joined to, or is associated with a stabilizing component. The coupler is allowed to interact with the polypeptide, then the stabilizing components are linked to form a tethering complex. In some embodiments, the linking of the stabilizing components can be controlled and/or inducible. In some cases, the linking of the stabilizing components does not occur until the stabilizing components are “activated”. For example, the stabilizing components are linked upon exposure to light. In some cases, the stabilizing components are linked upon introduction to a linking agent. For example, the linking agent comprises a chemical reagent, a biological reagent, or a combination thereof. In some embodiments, the linking agent comprises a protein or a polypeptide. In some embodiments, the linking agent comprises metal ions. In some embodiments, the linking agent comprises a heterobifunctional or homobifunctional crosslinking agent (Hermanson G, Bioconjugate Techniques, (2013) Academic Press). Once activated, the linking of the stabilizing components, either directly with each other or indirectly via a linker or other components, allows formation of a tethering complex connecting the coupler to a solid support.

In some embodiments, the linking of the stabilizing components (directly or indirectly) forms a tethering complex adequately or sufficiently stable for performing other steps of the amino acid identification or polypeptide sequencing. In some embodiments, the formation of the tethering complex is reversible, so the tethering complex is formed and then can be disassembled. In some embodiments, the method for performing a tethering reaction is temporally controlled. In some embodiments, the linking of the stabilizing components is inducible. In some embodiments, the method for performing a tethering reaction between two stabilizing components includes an activation step for the tethering complex to form. In some embodiments, the method for performing a tethering reaction includes an activation step for linking the stabilizing components. For example, the linking of the stabilizing components can involve photosensitive step (e.g. photoisomerization) or can involve hybridization-based interactions. In some cases, the stabilizing components comprise caged compounds or caged molecules, such as small organic molecules. In some cases, the stabilizing component is a photosensitive caged molecule. In some embodiments, the provided methods may provide the advantage of providing stability and extra control in forming the complex comprising the coupler and the polypeptide, allowing for efficient attachment to the solid support and subsequent release. In some embodiments, the relative affinity of stabilizing components to each other and/or to the linking agent is at least as high as the affinity of the coupler to the polypeptide. In some cases, the method includes a wash step after allowing the coupler to interact with the binding site located on the polypeptide. The wash step may remove non-specific binding of the coupler to non-target molecules. In some cases, the linking agent for linking the stabilizing components is provided and introduced after the wash step.

The formation of the tethering complex can be accomplished by a number of different ways depending on the design of the components in the complex. For example, the coupler may be joined to a stabilizing component by a linker of various lengths and the distance between the components may vary. In some embodiments, the polypeptide is associated or joined to a stabilizing component via a linker of various lengths based on the interaction of the components in the complex.

In some embodiments, the immobilized polypeptide is associated with a first stabilizing component and the coupler is associated with a second stabilizing component. In some aspects, the first and second stabilizing components are the same or different. The coupler or polypeptide may be directly associated with, joined to, attached to the stabilizing component(s). The coupler or polypeptide may be indirectly associated with, joined to, attached to the stabilizing component(s), such as via a linker of various lengths and flexibility (e.g., PEG linker of different length or another flexible linker). In some embodiments, the polypeptide is joined to a nucleic acid molecule (e.g., a recording tag) that is joined to the stabilizing component via a linker (e.g., PEG linker). In some embodiments, the polypeptide is joined to a bait nucleic acid molecule which hybridizes with at least a portion of the capture nucleic acid molecule that is immobilized on a solid support and the capture nucleic acid molecule is joined to the stabilizing component. In certain embodiments, a linker joins two molecules (polypeptide and stabilizing component) via enzymatic reaction or chemistry reaction (e.g., click chemistry). In some embodiments, the stabilizing components are joined to the polypeptide or coupler via a functional moiety, such as a click chemistry moiety, an aldehyde, an azide/alkyne, a maleimide/thiol, an epoxide/nucleophile, an inverse electron demand Diels-Alder (iEDDA) group, or a moiety for a Staudinger reaction. In some embodiments, the stabilizing components are joined to the polypeptide or coupler via hybridization of attached nucleic acid molecules or oligonucleotides.

In some embodiments, a stabilizing component is joined or attached (directly or indirectly via a linker) to a nucleic acid molecule or oligonucleotide. For example, the nucleic acid molecule or oligonucleotide joined or associated with the stabilizing component is configured for hybridization to a complementary nucleic acid molecule or oligonucleotide associated with the polypeptide (e.g. via the capture nucleic acid/recording tag). In some embodiments, the complementary nucleic acid molecule or oligonucleotide is associated or joined to a binding molecule or a binding pair member such as a biotin.

In some embodiments, a recording tag is joined to a binding pair member, e.g., a biotin molecule (or similar molecule) at the 5′ end. In some embodiments, a recording tag is joined to a stabilizing component DNA and the stabilizing component can be associated with its complementary stabilizing component nucleic acid which is joined to a binding pair member, e.g., a biotin (or similar molecule such as iEDDA group such methyl tetrazine (mTet) or trans-cyclooctene (TCO)). In some embodiments, a coupler is joined via a linker to a coding tag which is joined via a linker to a biotin molecule. In some embodiments, the stabilizing component can be associated with its complementary stabilizing component nucleic acid which is joined to a biotin (or similar molecule) (FIG. 2C). In some embodiments, the interaction of the stabilizing components with each other or with the linking agent is covalent or non-covalent.

The stabilizing components can be joined to the coupler and polypeptide using standard conjugation chemistries (Hermanson G, Bioconjugate Techniques, (2013) Academic Press). A variety of binding partners or pairs are known to those of skill in the art and may be used in the subject tethering reactions to stabilize the interaction between the stabilizing components. Selection of the stabilizing component may be based on affinity of the stabilizing components to each other or for the linking agent, speed of interaction, strength of the interaction, reversibility of the interaction, etc. In some embodiments, the stabilizing components each comprises a biological molecule, a chemical molecule, a small molecule or a combination thereof. In some embodiments, the stabilizing components comprises any appropriate binding partners, host-guest molecules or motifs, other interacting molecules, or portions thereof. See e.g. Liu et al., Chem Soc Rev. (2017); 46(9): 2391-2403; Mantooth et al., Macromol Biosci. (2019) 19(1):e1800281. Exemplar host-guest interactions include the supramolecular cyclic cucurbit[N]uril (N=5-8) host molecules which interact, in a reversible manner, with guest molecules with extremely high affinity (Ka˜ 10\hat{ }12 to 10\hat{ }15). For instance, cucurbit[7]uril rapidly (within minutes) forms host-guest complexes with ferrocene or adamantane derivatives with an binding affinity of 10\hat{ }12-10\hat{ }13, respectively (Barrow, S. J., et al. (2015). “Cucurbituril-Based Molecular Recognition.” Chem Rev 115(22): 12320-12406, incorporated by reference herein). In some aspects, the stabilizing component comprises an organic molecule or a synthetic molecule. In some embodiments, the stabilizing component is or comprises a small molecule, a compound, a protein, a protein complex, polypeptide, peptide, nucleic acid molecule, carbohydrate, lipid, macrocycle, a chimeric polypeptide, a synthetic host, or any combinations thereof. In some embodiments, the stabilizing component is or comprises an antibody, a catalytic antibody, an antigen, an enzyme, an inhibitor, a ligand, a protein, a substrate, or an organic compound. In some embodiments, the stabilizing component is or comprises a hapten. A hapten molecule may be attached at different positions in the hapten molecule to the binding agent or the polypeptide (or an associated polynucleotide or nucleic acid molecule).

In some embodiments, at least one of the stabilizing components may comprise a photosensitive molecule (e.g. photolabile or photoisomerization). In some embodiments, the stabilizing components are configured for nucleic acid hybridization-based interactions. In some cases, the stabilizing components comprise or are associated with caged compounds or caged molecules, such as small organic molecules. In some other embodiments, the stabilizing components comprise or are associated with one or more components of a known host-guest interaction.

In some embodiments, the introduction of the light, activating the stabilizing component, or providing the linking agent provides temporal control over the linking of the stabilizing components. In some embodiments, the stabilizing components are linked to each other (directly or indirectly) upon introduction of a linking agent or light. In some embodiments, the stabilizing components remain inactive, or are generally not linked to each other or to a linking agent until activated. In some cases, activation may refer to the introduction of a molecule, photoactivation (e.g., introduction of light, for example, UV or blue light), change in pH of the reaction, change in condition of the reaction (e.g., change in temperature), or destruction or removal of inhibition (e.g., uncaging of a molecule). In some aspects, upon activation, one or more of the stabilizing components undergoes a conformational change. In some cases, one or more of the stabilizing components is under allosteric control and upon activation (e.g., by binding to a linking agent), the stabilizing component is made available for interactions/binding. In some embodiments, to form the tethering complex, the light or linking agent induces uncaging of one or both of the stabilizing components, deblocking of one or both of the stabilizing components, isomerization of the stabilizing components, hybridization of the stabilizing components, and/or binding of the stabilizing components. In some embodiments, once activated, the linking of the stabilizing components occurs in less than about 10 seconds, less than about 30 seconds, less than about 60 seconds, less than about 80 seconds, less than about 100 seconds, less than about 2 minutes, less than about 5 minutes, less than about 10 minutes, or less than about 15 minutes. It may be desirable to select stabilizing components that may be linked in an amount of time less than the time for the binding agent to dissociate from the target, to maintain specificity of the binding agent with the target.

In some embodiments, linking of the stabilizing components is specific or occurs within the complex between a stabilizing component associated with the coupler and a stabilizing component associated with the target bound by said coupler. For example, the method is performed such that linking of stabilizing components is not intermolecular, e.g., between stabilizing components of different polypeptides. It may be preferred that linking does not occur between a stabilizing component of the coupler bound to the polypeptide and a stabilizing component of a different polypeptide. In some aspects, linking of intramolecular stabilizing components within a complex can be achieved by titrating or controlling the density of polypeptides on a support or within the volume of a support. In some cases, the control of density of the polypeptides is performed by controlling the density of functional coupling groups for attaching the polypeptides or by spiking a competitor or “dummy” reactive molecule when immobilizing the polypeptides to the support.

In some embodiments, the linking agent comprises a chemical reagent, a non-biological reagent, a biological reagent, or a combination thereof. In some cases, the linking agent comprises one or more proteins. In some embodiments, the stabilizing components are linked upon a change in pH of the reaction or reaction mixture or environment. In some embodiments, the linking agent comprises at least one polynucleotide or nucleic acid comprising a sequence which hybridizes to at least one of the stabilizing components. In some particular embodiments, the linking agent comprises a polynucleotide comprising two hybridization regions: one region for hybridizing to a polynucleotide of the first stabilizing component and one region for hybridizing to a polynucleotide of the second stabilizing component. In some embodiments, the stabilizing component comprises a biotin or an analog thereof (e.g. desthiobiotin) and the linking agent is or comprises an avidin (e.g., streptavidin or neutravidin). In some embodiments, each of the biotin or desthiobiotin may use any similar molecule or analog, depending on desired strength of the interaction. In another particular embodiment, the first stabilizing component is or comprises a first antibody or an antigen-recognizing fragment thereof; the second stabilizing component is or comprises a second antibody or an antigen-recognizing fragment thereof recognizing a different epitope from the first antibody; and the linking agent comprises two epitopes joined together and recognized by the first and second antibodies, so after introduction of the linking agent a tethering complex is formed comprising the first and second antibodies (or antigen-recognizing fragments thereof) and the linking agent. Alternatively, the linking agent can be a conjugate of two (different or identical) antigen-recognizing fragments, and the first and second stabilizing components comprise the corresponding antigen(s) that can bind antigen-recognizing fragments. In other examples, the linking agent can be a conjugate of two ligand-binding polypeptides joined together, or a dimer of a ligand-binding polypeptide, and the first and second stabilizing components comprise the corresponding ligand(s) that bind to the ligand-binding polypeptide(s). In yet other examples, the linking agent can be a conjugate of two ligands joined together, and the first and second stabilizing components comprise ligand-binding polypeptides that bind to the corresponding ligands. In some embodiments, the ligand-binding polypeptides can be functional fragments of ligand-binding proteins.

The following illustrates an exemplary workflow for performing a tethering reaction: a large collection of polypeptides (e.g., 50 million-1 billion or more) from a proteolytic digest of a biological sample is immobilized randomly on a solid support via DNA hybridization (e.g., beads) at an appropriate intermolecular spacing with nucleic acid capture molecules; the polypeptides are joined to nucleic acid capture molecules comprising the recording tag, which are each joined to a desthiobiotin molecule (as a first stabilizing component); the coupler comprising a biotin molecule (as a second stabilizing component) is contacted with the polypeptides; a wash is preformed to remove unreacted coupler; streptavidin is added to the reaction as a linking agent and associates with the biotin and desthiobiotin; a streptavidin molecule binds a biotin joined to the binding agent and a desthiobiotin joined to the target, thereby forming a tethering complex containing the coupler and the polypeptide. After that, the coupler-NTAA complex is cleaved from the polypeptide, but remains to be attached to the solid support via tethering complex. Then, binding agents are added that are configured to bind the tethered coupler-NTAA complex; the binding agents each joined to an associated nucleic acid molecule (coding tag) containing information regarding the binding agent. Next, binding of the binding agent to the tethered coupler-NTAA complex brings the coding tag of the binding agent in proximity to the recording tag associated with the polypeptide, so a transfer of the information from the coding tag to the recording tag occurs generating an extended recording tag. After information transfer, the stabilizing components, the bound binding agent with the coding tag, and the tethered coupler-NTAA can be removed using alkaline stripping conditions, and all the steps can be repeated with a newly exposed NTAA of the polypeptide. Finally, the extended recording tags are sequenced and the information is decoded to identify terminal amino acid(s) of the polypeptide.

In one embodiment, the first stabilizing component is the same as the second stabilizing component. For example, in the exemplary workflow from the previous paragraph, a biotin molecule can be used instead of desthiobiotin molecule, and two biotin molecules will interact with the linking agent and form the tethering complex. In another embodiment, the first stabilizing component has a lower affinity to the linking agent in comparison to an affinity of the second stabilizing component to the linking agent as shown in the exemplary workflow from the previous paragraph. In some embodiment, it will be preferable to use this combination of different stabilizing components such as desthiobiotin (DSB) and biotin. The use of a rapid high-affinity stabilizing component on the binding agent (biotin) and a lower affinity stabilizing component (DSB) associated with a target polypeptide provides for both rapid formation of the tethering complex and controllable release (disruption of the tethering complex) of the coupler, for example, by elution with biotin, which opens the target polypeptide for the next identification cycle to achieve reversible stabilization of the binding reaction.

In some embodiments, formation of the tethering complex is reversible and no covalent bonds are formed during formation of the tethering complex. Preferably, only non-covalent interactions are involved in the formation of the tethering complex. Examples of non-covalent interactions are electrostatic, 7-effects, van der Waals forces, formation of hydrogen bonds or other types of dipole-dipole interactions, hydrophobic interactions. In some embodiments, the disrupting is conducted by removing the linking agent. In some cases, the disrupting is conducted by introducing a destabilizing agent. For example, the destabilizing agent comprises heat, a denaturing agent, an enzyme, a competitor molecule, or a combination thereof. In some cases, the competitor molecule is a competitor for binding of or to the binding agent, the linking agent, and/or the stabilizing components. In other embodiments, reversible covalent bonds can be formed during formation of the tethering complex.

In some embodiments formation of the tethering complex is reversible via use of hybridization of the first stabilizing component to the capture DNA/recording tag complex associated with the polypeptide. Hybridization is reversible by using methods known in the art such as heat, low salt, chemical denaturants (e.g. alkaline reagents like NaOH, formamide, and DMSO) or some combination thereof. In a preferred embodiment, the tethering complex is dissociated using 0.1-1 N NaOH as a denaturant.

In some aspects, the disrupting the tethering complex allows the stabilizing component (e.g., associated with the polypeptide) to become available for interacting. In some cases, the method includes a repeated cycle of forming a tethering complex and disrupting the tethering complex such that the coupler is released from the target (polypeptide), allowing the target to be available for other reactions or treatments.

In some embodiments, the first stabilizing component associated with the polypeptide has a lower affinity to the linking agent in comparison to an affinity of the second stabilizing component to the linking agent. This setup allows for efficient disruption of the tethering complex and the coupler dissociation. Several types of stabilizing components can be utilized in this setup. One particular type includes using dethiobiotin (DSB) and biotin linked via streptavidin during the tethering complex formation, and then using biotin for dissociation. Other linking agents can also be used, preferably ones that have affinity sites for two different interacting partners. These partners can be included as stabilizing components and can be linked together upon introduction of the linking agent.

In some embodiments, one or more of the stabilizing components are cleavable. In some embodiments, two different cleavable stabilizing components (e.g., haptens) are attached to the target and coupler respectively, directly or indirectly via a nucleic acid molecule. Specific cleaving agents (e.g. chemical reagent for cleaving) can be used to cleave one stabilizing component while leaving the other stabilizing component intact. For example, the method may include using linking the stabilizing components to form a tethering complex comprising the coupler, the target and the stabilizing components, then cleaving the stabilizing component associated with the coupler while the stabilizing component remains associated with the polypeptide.

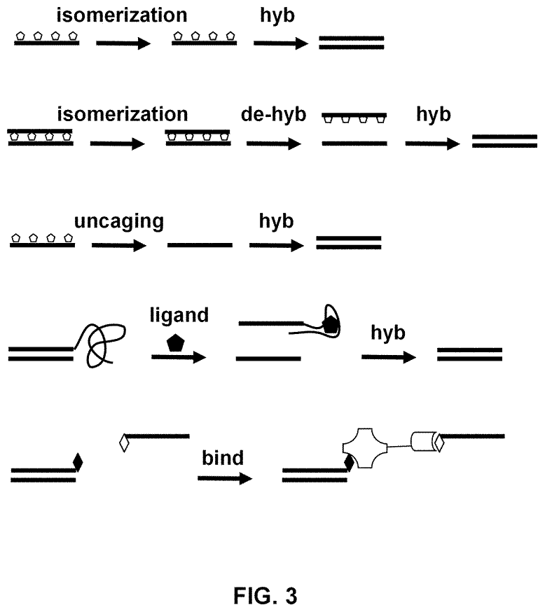

In some embodiments, the first or second stabilizing component comprises a polynucleotide, and the linking agent comprises a linking polynucleotide that hybridizes to the polynucleotide of one of the stabilizing components. In some embodiments, known approaches can be used to generate controllable hybridization of two polynucleotides that will result in formation of the tethering complex containing the coupler and polypeptide. Several potential embodiments of controllable hybridization of two polynucleotides (used as stabilizing components) are illustrated in FIG. 3. For example, photoisomerization or uncaging can trigger hybridization, as disclosed in Szymanski W, et al., Reversible photocontrol of biological systems by the incorporation of molecular photoswitches. Chem Rev. 2013 Aug. 14; 113(8):6114-78; Asanuma H, et al., Synthesis of azobenzene-tethered DNA for reversible photo-regulation of DNA functions: hybridization and transcription. Nat Protoc. 2007; 2(1):203-12; Yunqi Yan et al., Photocontrolled DNA hybridization stringency with fluorescence detection in heterogeneous assays, ACS Sens. 2016, 1, 5, 566-571; Goldau T, et al., Azobenzene C-Nucleosides for Photocontrolled Hybridization of DNA at Room Temperature. Chemistry. 2015 Dec. 1; 21(49):17870-6; Menge C, Heckel A. Coumarin-caged dG for improved wavelength-selective uncaging of DNA. Org Lett. 2011 Sep. 2; 13(17):4620-3; Ruble B K, et al., Caged oligonucleotides for studying biological systems, J Inorg Biochem. 2015 September; 150: 182-188; Adam V, et al., Expanding the Toolbox of Photoswitches for DNA Nanotechnology Using Arylazopyrazoles. Chemistry. 2018 Jan. 24; 24(5):1062-1066, which are incorporated herein by reference. “Caged” compounds have inactivating groups bonded to bioactive molecules that can be readily removed in an orthogonal manner, for example, by UV light or visible light photoirradiation. By using light to turn on activity, high spatial and temporal control of polynucleotide hybridization can be attained.

The following embodiment illustrates another exemplary workflow including a tethering reaction: a large collection of polypeptides (e.g., 1 million, 10 millions, 100 millions, 1 billion or more) from a proteolytic digest are immobilized randomly on a substrate (e.g., beads) at an appropriate intermolecular spacing with nucleic acid capture molecules. The methods disclosed herein are designed for high throughput identification of peptides, and at least 200, at least 500, at least 1000, at least 2000, at least 5000, or at least 1000 different polypeptides can be analyzed in parallel (in a single assay). The target polypeptides are joined to nucleic acid capture molecules which are each joined to a hybridizable polynucleotide (the first stabilizing component); the coupler joined to a complementary hybridizable polynucleotide (the second stabilizing component) is contacted with the target polypeptides and allowed to interact; a wash is preformed to remove non-specific binding. The hybridizable polynucleotide is modified by introducing photoswitchable nucleotides or caged nucleotides to prevent hybridization with its complementary polynucleotide. Light of a certain wavelength is introduced to the reaction as a linking agent, inducing uncaging of nucleotides and allowing hybridization and formation of a tethering complex containing the coupler and polypeptide. Several caged or modified nucleotide variants can be used. First, diethylaminocoumarin (DEACM) as a photoremovable protecting group for 2′-deoxyguanosine can be used, and light with 405 nm wavelength can be used for uncaging as disclosed in Menge C, Heckel A. Coumarin-caged dG for improved wavelength-selective uncaging of DNA. Org Lett. 2011 Sep. 2; 13(17):4620-3. Second, azobenzene moieties can be introduced into certain DNA nucleotides on a conventional DNA synthesizer using a phosphoramidite monomer bearing an azobenzene synthesized from D-threoninol as disclosed in Asanuma H, et al., Synthesis of azobenzene-tethered DNA for reversible photo-regulation of DNA functions: hybridization and transcription. Nat Protoc. 2007; 2(1):203-12. Hybridization of a polynucleotide having azobenzene-modified DNA can be reversibly photo-controlled by controlling cis-trans isomerization of the azobenzene. The hybridization can be photo-induced by cis-trans isomerization of the azobenzene moiety by irradiation of a visible light (wavelength is more than 400 nm). When azobenzene is in a trans-form, a stable duplex can be formed with a complementary strand. Importantly, hybridization is reversible and can be disrupted by UV light irradiation (wavelength between 300 nm and 400 nm), which induces isomerization of the trans-azobenzene to its cis-form. Thus, several cycles of formation and disruption of the tethering complex containing binding agent and target polypeptide can be achieved. In addition to azobenzenes, other known groups that undergo photo-induced structural switches include stilbenes, hemithioindigos, spiropyrans, diarylethenes and fulgides (Szymanski W, et al., Reversible photocontrol of biological systems by the incorporation of molecular photoswitches. Chem Rev. 2013 Aug. 14; 113(8):6114-78). Photoswitchable units can be introduced to nucleotide monophosphates in nucleic acid oligomers via two methods: alkylation of a thiophosphate-modified backbone and amidation of the ribose moiety on a 2′-aminodeoxyuridylate analog as disclosed in Szymanski W, et al., Reversible photocontrol of biological systems by the incorporation of molecular photoswitches. Chem Rev. 2013 Aug. 14; 113(8):6114-78 and references therein.

In some embodiments, the linking agent comprises a metal ion that links two stabilizing components together. One particular example of such embodiment is described in Nakamura T, et al., A metal-ion-responsive adhesive material via switching of molecular recognition properties. Nat Commun. 2014 Aug. 7; 5:4622, where divalent metal ions (Fe²⁺, Co²⁺, Ni²⁺, Cu²⁺, Zn²⁺) are used specifically for adherence of two hydrogels. Metal ions can bring together spatially separated metal-chelating or metal-coordinating groups to form a tethering complex having a metal ion in its center.

The methods provided herein describe a tethering reaction with a coupler and a target (polypeptide). Prior to performing the tethering reaction, a target may be obtained from a source and treated in various ways to prepare the target for the tethering reaction, such as by joining to a stabilizing component. The tethering reaction may be performed on a plurality of targets. In some embodiments, the target is immobilized on a support. In some cases, the targets are polypeptide from a mixture of polypeptides obtained from a sample. In some embodiments, the sample comprises but is not limited to, mammalian or human cells, yeast cells, and/or bacterial cells. In some embodiments, the sample contains cells that are from a sample obtained from a multicellular organism. For example, the sample may be isolated from an individual. In some embodiments, the sample may comprise a single cell type or multiple cell types. In some embodiments, the sample may be obtained from a mammalian organism or a human, for example by puncture, or other collecting or sampling procedures. In some embodiments, the sample comprises two or more cells. In some embodiments, the biological sample may contain whole cells and/or live cells and/or cell debris. In some embodiments, a suitable source or sample, may include but is not limited to: biological samples, such as biopsy samples, cell cultures, cells (both primary cells and cultured cell lines), sample comprising cell organelles or vesicles, tissues and tissue extracts; of virtually any organism. For example, a suitable source or sample, may include but is not limited to: biopsy; fecal matter; bodily fluids (such as blood, whole blood, serum, plasma, urine, lymph, bile, aqueous humor, breast milk, cerumen (earwax), chyle, chyme, endolymph, perilymph, exudates, cerebrospinal fluid, interstitial fluid, aqueous or vitreous humor, colostrum, sputum, amniotic fluid, saliva, anal and vaginal secretions, gastric acid, gastric juice, lymph, mucus (including nasal drainage and phlegm), pericardial fluid, peritoneal fluid, pleural fluid, pus, rheum, saliva, sebum (skin oil), sputum, synovial fluid, perspiration and semen, a transudate, vomit and mixtures of one or more thereof, an exudate (e.g., fluid obtained from an abscess or any other site of infection or inflammation) or fluid obtained from a joint (normal joint or a joint affected by disease such as rheumatoid arthritis, osteoarthritis, gout or septic arthritis) of virtually any organism, with mammalian-derived samples, including microbiome-containing samples, being preferred and human-derived samples, including microbiome-containing samples, being particularly preferred; environmental samples (such as air, agricultural, water and soil samples); microbial samples including samples derived from microbial biofilms and/or communities, as well as microbial spores; tissue samples including tissue sections, research samples including extracellular fluids, extracellular supernatants from cell cultures, inclusion bodies in bacteria, cellular components including mitochondria and cellular periplasm. In some embodiments, the biological sample comprises a body fluid or is derived from a body fluid, wherein the body fluid is obtained from a mammal or a human. In some embodiments, the sample includes bodily fluids, or cell cultures from bodily fluids. In certain embodiments, a peptide, polypeptide, or protein can be fragmented. Peptides, polypeptides, or proteins can be fragmented by any means known in the art, including fragmentation by a protease or endopeptidase. In some embodiments, fragmentation of a peptide, polypeptide, or protein is targeted by use of a specific protease or endopeptidase. A specific protease or endopeptidase binds and cleaves at a specific consensus sequence (e.g., TEV protease). In other embodiments, fragmentation of a peptide, polypeptide, or protein is non-targeted or random by use of a non-specific protease or endopeptidase. Protein and polypeptide fragmentation into peptides can be performed before or after attachment of a DNA tag or DNA recording tag.

Chemical reagents can also be used to digest proteins into peptide fragments. A chemical reagent may cleave at a specific amino acid residue (e.g., cyanogen bromide hydrolyzes peptide bonds at the C-terminus of methionine residues). Chemical reagents for fragmenting polypeptides or proteins into smaller peptides include cyanogen bromide (CNBr), hydroxylamine, hydrazine, formic acid, BNPS-skatole [2-(2-nitrophenylsulfenyl)-3-methylindole], iodosobenzoic acid, •NTCB +Ni (2-nitro-5-thiocyanobenzoic acid), etc.

In certain embodiments, following enzymatic or chemical cleavage, the resulting peptide fragments are approximately the same desired length, e.g., from about 10 amino acids to about 70 amino acids, from about 10 amino acids to about 60 amino acids, from about 10 amino acids to about 50 amino acids, about 10 to about 40 amino acids, from about 10 to about 30 amino acids, from about 20 amino acids to about 70 amino acids, from about 20 amino acids to about 60 amino acids, from about 20 amino acids to about 50 amino acids, about 20 to about 40 amino acids, from about 20 to about 30 amino acids, from about 30 amino acids to about 70 amino acids, from about 30 amino acids to about 60 amino acids, from about 30 amino acids to about 50 amino acids, or from about 30 amino acids to about 40 amino acids. A cleavage reaction may be monitored, preferably in real time, by spiking the protein or polypeptide sample with a short test FRET (fluorescence resonance energy transfer) peptide comprising a peptide sequence containing a proteinase or endopeptidase cleavage site. In the intact FRET peptide, a fluorescent group and a quencher group are attached to either end of the peptide sequence containing the cleavage site, and fluorescence resonance energy transfer between the quencher and the fluorophore leads to low fluorescence. Upon cleavage of the test peptide by a protease or endopeptidase, the quencher and fluorophore are separated giving a large increase in fluorescence. A cleavage reaction can be stopped when a certain fluorescence intensity is achieved, allowing a reproducible cleavage endpoint to be achieved.

In some embodiments, a subset of proteins within a sample is fractionated such that a subset of the proteins is sorted from the rest of the sample. For example, the sample may undergo fractionation methods prior to attachment to a support. Alternatively, or additionally, protein enrichment methods may be used to select for a specific protein or peptide (see, e.g., Whiteaker et al., 2007, Anal. Biochem. 362:44-54, incorporated by reference in its entirety) or to select for a particular post translational modification (see, e.g., Huang et al., 2014. J. Chromatogr. A 1372:1-17, incorporated by reference in its entirety). Alternatively, a particular class or classes of proteins such as immunoglobulins, or immunoglobulin (Ig) isotypes such as IgG, can be affinity enriched or selected for analysis. Overly abundant proteins can also be subtracted from the sample using standard immunoaffinity methods. Depletion of abundant proteins can be useful for plasma samples where over 80% of the protein constituent is albumin and immunoglobulins. Several commercial products are available for depletion of plasma samples of overly abundant proteins, including depletion spin columns that remove top 2-20 plasma proteins (Pierce, Agilent), or PROTIA and PROT20 (Sigma-Aldrich).

In certain embodiments, a protein sample dynamic range can be modulated by fractionating the protein sample using standard fractionation methods, including electrophoresis and liquid chromatography (Zhou et al., 2012, Anal Chem 84(2): 720-734), or partitioning the fractions into compartments (e.g., droplets) loaded with limited capacity protein binding beads/resin (e.g. hydroxylated silica particles) (McCormick, 1989, Anal Biochem 181(1): 66-74) and eluting bound protein. Excess protein in each compartmentalized fraction is washed away. Examples of electrophoretic methods include capillary electrophoresis (CE), capillary isoelectric focusing (CIEF), capillary isotachophoresis (CITP), free flow electrophoresis, gel-eluted liquid fraction entrapment electrophoresis (GELFrEE). Examples of liquid chromatography protein separation methods include reverse phase (RP), ion exchange (IE), size exclusion (SE), hydrophilic interaction, etc. Examples of compartment partitions include emulsions, droplets, microwells, physically separated regions on a flat substrate, etc. Exemplary protein binding beads/resins include silica nanoparticles derivatized with phenol groups or hydroxyl groups (e.g., StrataClean Resin from Agilent Technologies, RapidClean from LabTech, etc.). By limiting the binding capacity of the beads/resin, highly-abundant proteins eluting in a given fraction will only be partially bound to the beads, and excess proteins removed.

In some embodiments, a partition barcode is used which comprises assignment of a unique barcode to a subsampling of polypeptides from a population of polypeptides within a sample. This partition barcode may be comprised of identical barcodes arising from the partitioning of polypeptides within compartments labeled with the same barcode (e.g., a barcoded bead population in which multiple beads share the same barcode). The use of physical compartments effectively subsamples the original sample to provide assignment of partition barcodes. For instance, a set of beads labeled with 10,000 different compartment barcodes is provided. Furthermore, suppose in a given assay, that a population of 1 million beads are used in the assay. On average, there are 100 beads per compartment barcode (Poisson distribution). Further suppose that the beads capture an aggregate of 10 million polypeptides. On average, there are 10 polypeptides per bead, with 100 compartments per compartment barcode, there are effectively 1,000 polypeptides per partition barcode (comprised of 100 compartment barcodes for 100 distinct physical compartments).

In another embodiment, single molecule partitioning and partition barcoding of polypeptides is accomplished by labeling polypeptides (chemically or enzymatically) with an amplifiable DNA UMI tag (e.g., recording tag) at the N or C terminus, or both. DNA tags are attached to the body of the polypeptide (internal amino acids) via non-specific photo-labeling or specific chemical attachment to reactive amino acids such as lysines. Information from the recording tag attached to the terminus of the peptide is transferred to the DNA tags via an enzymatic emulsion PCR (Williams et al., Nat Methods, (2006) 3(7):545-550; Schutze et al., Anal Biochem. (2011) 410(1):155-157) or emulsion in vitro transcription/reverse transcription (IVT/RT) step. In the preferred embodiment, a nanoemulsion is employed such that, on average, there is fewer than a single polypeptide per emulsion droplet with size from 50 nm-1000 nm (Nishikawa et al., J Nucleic Acids. (2012) 2012: 923214; Gupta et al., Soft Matter. (2016) 12(11):2826-41; Sole et al., Langmuir (2006, 22(20):8326-8332). Additionally, all the components of PCR are included in the aqueous emulsion mix including primers, dNTPs, Mg2+, polymerase, and PCR buffer. If IVT/RT is used, then the recording tag is designed with a T7/SP6 RNA polymerase promoter sequence to generate transcripts that hybridize to the DNA tags attached to the body of the polypeptide (Ryckelynck et al., RNA. (2015) 21(3):458-469). A reverse transcriptase (RT) copies the information from the hybridized RNA molecule to the DNA tag. In this way, emulsion PCR or IVT/RT can be used to effectively transfer information from the terminus recording tag to multiple DNA tags attached to the body of the polypeptide.

In some embodiments, a sample of polypeptide targets (e.g., peptides, polypeptides, or proteins) can be processed into a physical area or volume e.g., into a compartment. Various processing and/or labeling steps may be performed on the sample prior to performing the tethering reaction. In some embodiments, the compartment separates or isolates a subset of polypeptides from a sample of polypeptides. In some embodiments, the compartment may be an aqueous compartment (e.g., microfluidic droplet), a solid compartment (e.g., picotiter well or microtiter well on a plate, tube, vial, bead), or a separated region on a surface. In some cases, a compartment may comprise one or more beads to which polypeptides may be immobilized. In some embodiments, polypeptides in a compartment is labeled with a compartment tag including a barcode. For example, the polypeptides in one compartment can be labeled with the same barcode or polypeptides in multiple compartments can be labeled with the same barcode. See e.g., Valihrach et al., Int J Mol Sci. 2018 Mar. 11; 19(3). pii: E807. Encapsulation of cellular contents via gelation in beads is a useful approach to single cell analysis (Tamminen et al., Front Microbiol (2015) 6: 195; Spencer et al., ISME J (2016) 10(2): 427-436). Barcoding single cell droplets enables all components from a single cell to be labeled with the same identifier (Klein et al., Cell (2015) 161(5): 1187-1201; Zilionis et al., Nat Protoc (2017) 12(1): 44-73; International Patent Publication No. WO 2016/130704). Compartment barcoding can be accomplished in a number of ways including direct incorporation of unique barcodes into each droplet by droplet joining (Bio-Rad Laboratories), by introduction of barcoded beads into droplets (10× Genomics), or by combinatorial barcoding of components of the droplet post encapsulation and gelation using and split-pool combinatorial barcoding as described by Gunderson et al. (International Patent Publication No. WO 2016/130704, incorporated by reference in its entirety).

In some embodiments, the target (e.g., polypeptide) is joined to a support before performing the tethering reaction. In some cases, it is desirable to use a support with a large carrying capacity to immobilize a large number of polypeptides. In some embodiments, it is preferred to immobilize the targets using a three-dimensional support (e.g., a porous matrix or a bead). For example, the preparation of the polypeptides including joining the polypeptide to a support is performed prior to performing the tethering reaction. In some embodiments, the preparation of the polypeptides including joining the polypeptide to nucleic acid molecule or a oligonucleotide may be performed prior to or after immobilizing the polypeptide.

Various reactions may be used to attach the polypeptides to a support (e.g., a solid or a porous support). The polypeptides may be attached directly or indirectly to the support. In some cases, the polypeptides are attached to the support via a nucleic acid. Exemplary reactions include click chemistry reactions, such as the copper catalyzed reaction of an azide and alkyne to form a triazole (Huisgen 1,3-dipolar cycloaddition), strain-promoted azide alkyne cycloaddition (SPAAC), reaction of a diene and dienophile (Diels-Alder), strain-promoted alkyne-nitrone cycloaddition, reaction of a strained alkene with an azide, tetrazine or tetrazole, alkene and azide [3+2] cycloaddition, alkene and tetrazine inverse electron demand Diels-Alder (IEDDA) reaction (e.g., m-tetrazine (mTet) or phenyl tetrazine (pTet) and trans-cyclooctene (TCO)); or pTet and an alkene), alkene and tetrazole photoreaction, Staudinger ligation of azides and phosphines, and various displacement reactions, such as displacement of a leaving group by nucleophilic attack on an electrophilic atom (Horisawa 2014, Knall, Hollauf et al. 2014). Exemplary displacement reactions include reaction of an amine with: an activated ester; an N-hydroxysuccinimide ester; an isocyanate; an isothioscyanate, an aldehyde, an epoxide, or the like. In some embodiments, iEDDA click chemistry is used for immobilizing polypeptides to a support since it is rapid and delivers high yields at low input concentrations. In another embodiment, m-tetrazine rather than tetrazine is used in an iEDDA click chemistry reaction, as m-tetrazine has improved bond stability. In another embodiment, phenyl tetrazine (pTet) is used in an iEDDA click chemistry reaction. In one case, a polypeptide is labeled with a bifunctional click chemistry reagent, such as alkyne-NHS ester (acetylene-PEG-NHS ester) reagent or alkyne-benzophenone to generate an alkyne-labeled polypeptide. In some embodiments, an alkyne can also be a strained alkyne, such as cyclooctynes including Dibenzocyclooctyl (DBCO) and others.

In certain embodiments where multiple polypeptides are immobilized on the same support, the polypeptides can be spaced appropriately to accommodate methods of performing the tethering reaction and any downstream analysis steps to be used to assess the polypeptide. For example, it may be advantageous to space the polypeptides that optimally to allow a nucleic acid-based method for assessing and sequencing the polypeptides to be performed. In some embodiments, the method for assessing and sequencing protein targets involve a binding agent which binds to the isolated amino acid molecule of the polypeptide and the binding agent comprises a coding tag with information that is transferred to a nucleic acid attached to the polypeptide. In some cases, spacing of the polypeptides on the support is determined based on the consideration that information transfer from a coding tag of a binding agent bound to one coupler-amino acid may reach a neighboring polypeptide.

In some embodiments, the surface of the support is passivated (blocked). A “passivated” surface refers to a surface that has been treated with outer layer of material. Methods of passivating surfaces include standard methods from the fluorescent single molecule analysis literature, including passivating surfaces with polymer like polyethylene glycol (PEG) (Pan et al., 2015, Phys. Biol. 12:045006), polysiloxane (e.g., Pluronic F-127), star polymers (e.g., star PEG) (Groll et al., 2010, Methods Enzymol. 472:1-18), hydrophobic dichlorodimethylsilane (DDS)+self-assembled Tween-20 (Hua et al., 2014, Nat. Methods 11:1233-1236), diamond-like carbon (DLC), DLC+PEG (Stavis et al., 2011, Proc. Natl. Acad. Sci. USA 108:983-988), and zwitterionic moiety (e.g., U.S. Patent Application Publication US 2006/0183863). In addition to covalent surface modifications, a number of passivating agents can be employed as well including surfactants like Tween-20, polysiloxane in solution (Pluronic series), poly vinyl alcohol (PVA), and proteins like BSA and casein. Alternatively, density of polypeptides (e.g., proteins, polypeptide, or peptides) can be titrated on the surface or within the volume of a solid substrate by spiking a competitor or “dummy” reactive molecule when immobilizing the polypeptides or peptides to the solid substrate.

To control spacing of the immobilized polypeptides on the support, the density of functional coupling groups for attaching the target (e.g., TCO or carboxyl groups (COOH)) may be titrated on the substrate surface. In some embodiments, multiple polypeptides are spaced apart on the surface or within the volume (e.g., porous supports) of a support such that adjacent molecules are spaced apart at a distance of about 50 nm to about 500 nm, or about 50 nm to about 400 nm, or about 50 nm to about 300 nm, or about 50 nm to about 200 nm, or about 50 nm to about 100 nm. In some embodiments, multiple molecules are spaced apart on the surface of a support with an average distance of at least 50 nm, at least 60 nm, at least 70 nm, at least 80 nm, at least 90 nm, at least 100 nm, at least 150 nm, at least 200 nm, at least 250 nm, at least 300 nm, at least 350 nm, at least 400 nm, at least 450 nm, or at least 500 nm. In some embodiments, multiple molecules are spaced apart on the surface of a support with an average distance of at least 50 nm. In some embodiments, polypeptides are spaced apart on the surface or within the volume of a support such that, empirically, the relative frequency of inter- to intra-molecular events (e.g. transfer of information) is <1:10; <1:100; <1:1,000; or <1:10,000.

In some embodiments, the plurality of polypeptides is coupled on the support spaced apart at an average distance between two adjacent molecules which ranges from about 50 to 100 nm, from about 50 to 250 nm, from about 50 to 500 nm, from about 50 to 750 nm, from about 50 to 1,000 nm, from about 50 to 1,500 nm, from about 50 to 2,000 nm, from about 100 to 250 nm, from about 100 to 500 nm, from about 200 to 500 nm, from about 300 to 500 nm, from about 100 to 1000 nm, from about 500 to 600 nm, from about 500 to 700 nm, from about 500 to 800 nm, from about 500 to 900 nm, from about 500 to 1,000 nm, from about 500 to 2,000 nm, from about 500 to 5,000 nm, from about 1,000 to 5,000 nm, or from about 3,000 to 5,000 nm.

In some embodiments, appropriate spacing of the polypeptides on the support is accomplished by titrating the ratio of available attachment molecules on the substrate surface. In some embodiments, the substrate surface (e.g., bead surface) is functionalized with a carboxyl group (COOH) which is treated with an activating agent (e.g., activating agent is EDC and Sulfo-NHS). In some embodiments, the substrate surface (e.g., bead surface) comprises NHS moieties. In some embodiments, a mixture of mPEGₙ-NH₂ and NH₂-PEGₙ-mTet is added to the activated beads (wherein n is any number, such as 1-100). The ratio between the mPEG₃-NH₂ (not available for coupling) and NH₂-PEG₂₄-mTet (available for coupling) is titrated to generate an appropriate density of functional moieties available to attach the polypeptides on the substrate surface. In certain embodiments, the mean spacing between coupling moieties (e.g., NH₂-PEG₄-mTet) on the solid surface is at least 50 nm, at least 100 nm, at least 250 nm, or at least 500 nm. In some specific embodiments, the ratio of NH₂-PEGₙ-mTet to mPEG₃-NH₂ is about or greater than 1:1000, about or greater than 1:10,000, about or greater than 1:100,000, or about or greater than 1:1,000,000. In some further embodiments, the recording tag attaches to the NH₂-PEGₙ-mTet. In some embodiments, the spacing of the polypeptides on the support is achieved by controlling the concentration and/or number of available COOH or other functional groups on the support.

The provided methods for performing a tethering reaction between stabilizing components may be used in combination with an assay for analyzing the target, such as in a reaction between binding agent and target (isolated amino acid in complex with the coupler) that results in information transfer and finally identification of the isolated amino acid. In some embodiments, additional treatments and reactions may be performed with the target before or after the tethering reaction. In some cases, some of the additional reactions and treatments may be performed while the tethering complex is intact. In some cases, the target or plurality of targets is obtained from a sample and immobilized on a support (e.g., on a bead). In some embodiments, the tethering reaction is useful for identifying the target or a portion thereof, such as by using a binding agent with a known binding profile. In certain embodiments, the binding agent comprises one or more detectable labels.

In some aspects, the amino acid identification assay includes contacting the target with a binding agent capable of binding to the target, wherein the binding agent comprises a coding tag with identifying information regarding the binding agent; and transferring the information of the coding tag to a recording tag (associated with the target polypeptide) to generate an extended recording tag. In some aspects, for releasable attachment of the isolated amino acid to a solid support, a tethering complex is formed by linking the stabilizing components (associated or joined to the polypeptide and the coupler). In some further embodiments, transferring the information of the coding tag to the recording tag to extend the recording tag may be repeated one or more times. In some cases, the analysis assay is performed on immobilized target molecules bound a cognate binding agent (e.g., antibody), then transferring information from the coding tags of bound antibodies to the recording tag associated with the target.

Provided herein is a method for identifying a terminal amino acid of a polypeptide, comprising the steps of: (a) providing a polypeptide and an associated recording tag attached to a solid support; (b) contacting the polypeptide with a coupler, wherein the coupler binds to a terminal amino acid of the polypeptide to form a coupler-polypeptide complex;

(c) attaching the coupler complex to the solid support;
(d) cleaving the coupler-polypeptide complex from the polypeptide, thereby providing a coupler-amino acid complex attached to the solid support;
(e) contacting the coupler-amino acid complex with a binding agent capable of binding to the coupler-amino acid complex, wherein the binding agent comprises a coding tag with identifying information regarding the binding agent;
(f) transferring the information of the coding tag of the binding agent to the recording tag;
(g) analyzing the recording tag extended after information transfer, thereby identifying the terminal amino acid of the polypeptide.

In preferred embodiments, the steps are performed in the order: (a), (b), (c), (d), (e), (f), (g), optionally repeating steps (b) through (f) one or more times. In preferred particular embodiments, the method further comprises releasing the coupler-amino acid complex after contacting with the binding agent and transferring the information of the coding tag of the binding agent, e.g. after step (f). In these embodiments, repeating steps (b) through (f) occur after releasing the coupler-amino acid complex from the solid support. In some cases, the releasing the coupler-amino acid complex from the solid support occurs by disrupting the tethering complex that is formed prior the step of contacting with the binding agent (prior step (e)). The disrupting may be performed by introducing a destabilizing agent, such as heat, a denaturing agent, an enzyme or a competitor molecule. In some embodiments, the bound binding agent and annealed coding tag can be removed following transfer of the identifying information (e.g., primer extension) by using highly denaturing conditions (e.g., 0.1-0.2 N NaOH, 6M Urea, 2.4 M guanidinium isothiocyanate, 95% formamide, etc.). In some embodiments, instead of releasing the coupler-amino acid complex from the solid support, the method further comprises blocking the coupler-amino acid complex after step (f) to prevent further interaction with binding agents. Blocking can be achieved, for example, by providing a blocking agent that interacts tightly with the coupler-amino acid complex and provides a steric shield to prevent further interaction of the coupler-amino acid complex with binding agents from the next cycles. In these embodiments, repeating steps (b) through (f) occurs after blocking the coupler-amino acid complex.

### Binding Agents

The methods described herein use a binding agent capable of binding to the single amino acid in complex with the coupler. The binding reaction may be performed by contacting a single binding agent with a single complex, a single binding agent with a plurality of complexes (different amino acids in complex with the coupler), a plurality of binding agents with a single complex, or a plurality of binding agents to a plurality of complexes. In preferred embodiments, the plurality of binding agents includes a mixture of binding agents, and each binding agent is capable of specific binding to the coupler-amino acid complex.

A binding agent can be any molecule (e.g., peptide, polypeptide, protein, nucleic acid, carbohydrate, small molecule, and the like) capable of binding to a single amino acid in complex with the coupler. A binding agent can be a naturally occurring, synthetically produced, or recombinantly expressed molecule. In some embodiments, the scaffold used to engineer a binding agent can be from any species, e.g., human, non-human, transgenic. A binding agent may bind to the amino acid portion of the target, or to both amino acid portion and coupler portion. In some embodiments, the binding agent comprises an antibody, an antigen-binding antibody fragment, a single-domain antibody (sdAb), a recombinant heavy-chain-only antibody (VHH), a single-chain antibody (scFv), a shark-derived variable domain (vNARs), a Fv, a Fab, a Fab′, a F(ab′)2, a linear antibody, a diabody, an aptamer, a peptide mimetic molecule, a fusion protein, a reactive or non-reactive small molecule, or a synthetic molecule.

In certain embodiments, a binding agent may be designed to bind covalently. Covalent binding can be designed to be conditional or favored upon binding to the correct moiety. For example, an target and its cognate binding agent may each be modified with a reactive group such that once the target-specific binding agent is bound to the target, a coupling reaction is carried out to create a covalent linkage between the two. Non-specific binding of the binding agent to other locations that lack the cognate reactive group would not result in covalent attachment. In some embodiments, the coupler comprises a ligand that is capable of forming a covalent bond to a binding agent. In some embodiments, the coupler comprises a ligand group that is capable of covalent binding to a binding agent. Covalent binding between a binding agent and its target may allow for more stringent washing to be used to remove binding agents that are non-specifically bound, thus increasing the specificity of the assay. In some embodiment, the method further includes performing one or more wash steps. In some embodiments, the method includes a wash step after contacting the binding agent to the amino acid-coupler complex to remove non-specifically bound binding agents. In some embodiments, the method includes a wash step after linking the stabilizing reagents and forming the tethering complex. The stringency of the wash step may be tuned depending on the affinity of the binding agent to the target and/or the strength and stability of the complex formed.

In some embodiments, the binding reaction involves binding agents configured to provide specificity for binding of the binding agent to the amino acid-coupler complex. A binding agent may bind to a specific amino acid from the amino acid-coupler complex, and bind much less efficient or not bind at all to other amino acids from amino acid-coupler complexes. A binding agent may also preferably bind to a chemically modified or labeled amino acid in the amino acid-coupler complex. In certain embodiments, a binding agent may be a selective binding agent. As used herein, selective binding refers to the ability of the binding agent to preferentially bind to a specific ligand (e.g., amino acid or class of amino acids) relative to binding to a different ligand (e.g., amino acid or class of amino acids). Selectivity is commonly referred to as the equilibrium constant for the reaction of displacement of one ligand by another ligand in a complex with a binding agent. Typically, such selectivity is associated with the spatial geometry of the ligand and/or the manner and degree by which the ligand binds to a binding agent, such as by hydrogen bonding, hydrophobic binding, and Van der Waals forces (non-covalent interactions) or by reversible or non-reversible covalent attachment to the binding agent. It should also be understood that selectivity may be relative, and as opposed to absolute, and that different factors can affect the same, including ligand concentration. Thus, in one example, a binding agent selectively binds one of the twenty standard amino acids. In some embodiments, a binding agent binds to a particular group of amino acids in complex with the coupler, for example, binds to positively charged amino acids in complex with the coupler.

In some embodiments, the binding agent is partially specific or selective. In some aspects, the binding agent preferentially binds one or more amino acids. In some embodiments, a binding agent may bind to or is capable of binding to two or more of the twenty standard amino acids. For example, a binding agent may preferentially bind the amino acids A, C, and G over other amino acids. In some other examples, the binding agent may selectively or specifically bind more than one amino acid. In some specific examples, binding agents with different specificities can share the same coding tag. In some embodiments, selectivity of a binding agent need not be absolute to a specific amino acid, but could be selective to a class of amino acids, such as amino acids with polar or non-polar side chains, or with electrically (positively or negatively) charged side chains, or with aromatic side chains, or some specific class or size of side chains, and the like. In some embodiments, the ability of a binding agent to selectively bind a component of an amino acid-coupler complex is characterized by comparing binding abilities of binding agents. In some embodiments, a binding agent selective for non-polar side chains is compared to a binding agent selective for polar side chains. In some embodiments, a binding agent selective for a component of an amino acid-coupler complex exhibits at least 1×, at least 2×, at least 5×, at least 10×, at least 50×, at least 100×, or at least 500× more binding compared to a binding agent selective for a different component.

In a particular embodiment, the binding agent has a high affinity and high selectivity for the component of the amino acid-coupler complex. In particular, a high binding affinity with a low off-rate may be efficacious for information transfer between the coding tag and recording tag. In certain embodiments, a binding agent has a Kd of about <500 nM, <200 nM, <100 nM, <50 nM, <10 nM, <5 nM, <1 nM, <0.5 nM, or <0.1 nM. In a particular embodiment, the binding agent is added to the amino acid-coupler complex at a concentration >1×, >5×, >10×, >100×, or >1000× its Kd to drive binding to completion. For example, binding kinetics of an antibody to a single protein molecule is described in Chang et al., J Immunol Methods (2012) 378(1-2): 102-115. In a particular embodiment, the provided methods for performing a binding reaction are compatible with a binding agent with medium to low affinity for the amino acid-coupler complex. A binding agent may be engineered for high affinity for a particular amino acid, high specificity for a particular amino acid, or both. In some embodiments, binding agents can be developed through directed evolution of promising affinity scaffolds using phage display. In certain embodiments, a binding agent may bind to a post-translational modification of an amino acid.

As used herein, the terms antibody and antibodies are used in a broad sense, to include not only intact antibody molecules, for example but not limited to immunoglobulin A, immunoglobulin G, immunoglobulin D, immunoglobulin E, and immunoglobulin M, but also any immunoreactive component(s) of an antibody molecule or portion thereof that specifically bind to at least one epitope (antigen-recognizing fragments). An antibody may be naturally occurring, synthetically produced, or recombinantly expressed. An antibody may be a fusion protein. An antibody may be an antibody mimetic. Examples of antibodies include but are not limited to, Fab fragments, Fab′ fragments, F(ab′)2 fragments, single chain antibody fragments (scFv), miniantibodies, nanobodies, diabodies, crosslinked antibody fragments, Affibody™, nanobodies, single domain antibodies, DVD-Ig molecules, alphabodies, affimers, affitins, cyclotides, molecules, and the like. Antigen-recognizing fragments derived from antibody using antibody engineering or protein engineering techniques are also used herein within the meaning of the term antibodies. Detailed descriptions of antibody and/or protein engineering, including relevant protocols, can be found in, among other places, J. Maynard and G. Georgiou, 2000, Ann. Rev. Biomed. Eng. 2:339-76; Antibody Engineering, R. Kontermann and S. Dubel, eds., Springer Lab Manual, Springer Verlag (2001); U.S. Pat. No. 5,831,012; and S. Paul, Antibody Engineering Protocols, Humana Press (1995).

As with antibodies, nucleic acid and peptide aptamers that specifically recognize an amino acid in complex with the coupler can be produced using known methods. Aptamers bind target molecules in a highly specific, conformation-dependent manner, typically with very high affinity, although aptamers with lower binding affinity can be selected if desired. Aptamers have been shown to distinguish between targets based on very small structural differences such as the presence or absence of a methyl or hydroxyl group and certain aptamers can distinguish between D- and L-enantiomers. Aptamers have been obtained that bind small molecular targets, including drugs, metal ions, and organic dyes, peptides, biotin, and proteins, including but not limited to streptavidin, VEGF, and viral proteins. Aptamers which specifically bind arginine and AMP have been described as well (see, Patel and Suri, 2000, J. Biotech. 74:39-60). Oligonucleotide aptamers that bind to a specific amino acid have been disclosed in Gold et al. (1995, Ann. Rev. Biochem. 64:763-97). RNA aptamers that bind amino acids have also been described (Ames and Breaker, 2011, RNA Biol. 8; 82-89; Mannironi et al., 2000, RNA 6:520-27; Famulok, 1994, J. Am. Chem. Soc. 116:1698-1706).

A binding agent can be made by modifying naturally-occurring or synthetically-produced proteins by genetic engineering to introduce one or more mutations in the amino acid sequence to produce engineered proteins that bind to a specific component of the amino acid-coupler complex. For example, exopeptidases (e.g., aminopeptidases, carboxypeptidases), exoproteases, mutated exoproteases, mutated anticalins, mutated ClpSs, antibodies, or tRNA synthetases can be modified to create a binding agent that selectively binds to a particular amino acid in complex with the coupler.

In some embodiments, the binding agent comprises an engineered carboxypeptidase. Carboxypeptidases can be modified to create a binding agent that selectively binds to a particular amino acid in complex with the coupler. A binding agent can also be designed or modified, and utilized, to specifically bind a modified amino acid in complex with the coupler, for example one that has a post-translational modification (e.g., phosphorylated amino acid). Strategies for directed evolution of proteins are known in the art (e.g., Yuan et al., 2005, Microbiol. Mol. Biol. Rev. 69:373-392), and include phage display, ribosomal display, mRNA display, CIS display, CAD display, emulsions, cell surface display method, yeast surface display, bacterial surface display, etc. In this manner, the binding agent may be engineered to selectively bind the combination of the coupler and the amino acid. For example, the NTAA of the polypeptide may be reacted with phenylisothiocyanate (PITC) to form a phenylthiocarbamoyl-NTAA derivative. After cleavage, a phenylthiocarbamoyl-amino acid derivative can be used for reaction with the binding agent, which may be fashioned to selectively bind both the phenyl group of the phenylthiocarbamoyl moiety as well as the alpha-carbon R group of the amino acid. Use of PITC in this manner allows for cleavage of the NTAA by Edman degradation as discussed below. In another embodiment, the NTAA may be reacted with Sanger's reagent (DNFB), to generate a DNP-labeled NTAA. Optionally, DNFB is used with an ionic liquid such as 1-ethyl-3-methylimidazolium bis[(trifluoromethyl)sulfonyl]imide ([emim][Tf2N]), in which DNFB is highly soluble. The addition of the DNP moiety provides a larger “handle” for the interaction of the binding agent with the amino acid, and should lead to a higher affinity interaction.

In yet another embodiment, a binding agent may be a modified aminopeptidase. In some embodiments, the binding agent may be a modified aminopeptidase that has been engineered to recognize the DNP-labeled amino acid providing cyclic control of aminopeptidase binding of the polypeptide. In one example, once the DNP-labeled NTAA is cleaved, another cycle of DNFB derivatization is performed in order to bind and cleave the newly exposed NTAA. In preferred particular embodiment, the aminopeptidase is a monomeric metalloprotease, such an aminopeptidase activated by zinc (Calcagno et al., Appl Microbiol Biotechnol. (2016) 100(16):7091-7102). In another example, a binding agent is derived from amino acid binding periplasmic binding proteins (PBPs) that demonstrate exquisite specificity towards particular amino acids, such as glutamate/aspartate binding protein (Hu Y, et al., Crystal structure of a glutamate/aspartate binding protein complexed with a glutamate molecule: structural basis of ligand specificity at atomic resolution. J Mol Biol. 2008 Sep. 26; 382(1):99-111) or histidine binding protein (Paul S, et al., Ligand binding specificity of the Escherichia coli periplasmic histidine binding protein, HisJ. Protein Sci. 2017 February; 26(2):268-279). PBP scaffolds may be evolved using phage display to recognize an amino acid in complex with the coupler.

In another example, highly-selective engineered ClpSs have also been described in the literature. Emili et al. describe the directed evolution of an E. coli ClpS protein via phage display, resulting in four different variants with the ability to selectively bind NTAAs for aspartic acid, arginine, tryptophan, and leucine residues (U.S. Pat. No. 9,566,335, incorporated herein). In one embodiment, the binding moiety of the binding agent comprises a member of the evolutionarily conserved ClpS family of adaptor proteins involved in natural N-terminal protein recognition and binding or a variant thereof (See e.g., Schuenemann et al., (2009) EMBO Reports 10(5); Roman-Hernandez et al., (2009) PNAS 106(22):8888-93; Guo et al., (2002) JBC 277(48): 46753-62; Wang et al., (2008) Molecular Cell 32: 406-414).

In certain embodiments, the binding agent further comprises one or more detectable labels such as fluorescent labels, in addition to the binding moiety. In some embodiments, the binding agent does not comprise a polynucleotide such as a coding tag. Optionally, the binding agent comprises a synthetic or natural antibody. In one embodiment, the binding agent comprises a polypeptide, such as a modified member of the ClpS family of adaptor proteins, such as a variant of an E. coli ClpS binding polypeptide, and a detectable label. In one embodiment, the detectable label is optically detectable. In some embodiments, the detectable label comprises a fluorescently moiety, a color-coded nanoparticle, a quantum dot or any combination thereof. In one embodiment the label comprises a polystyrene dye encompassing a core dye molecule such as a FluoSphere™, Nile Red, fluorescein, rhodamine, derivatized rhodamine dyes, such as TAMRA, phosphor, polymethadine dye, fluorescent phosphoramidite, TEXAS RED, green fluorescent protein, acridine, cyanine, cyanine 5 dye, cyanine 3 dye, 5-(2′-aminoethyl)-aminonaphthalene-1-sulfonic acid (EDANS), BODIPY, 120 ALEXA or a derivative or modification of any of the foregoing.

In a particular embodiment, anticalins are engineered for both high affinity and high specificity to amino acids in complex with the coupler. Certain varieties of anticalin scaffolds have suitable shape for binding single amino acids, by virtue of their beta barrel structure. An single amino acid in complex with the coupler can potentially fit and be recognized in this “beta barrel” bucket. High affinity anticalins with engineered novel binding activities have been described (reviewed by Skerra, 2008, FEBS J. 275: 2677-2683). For example, anticalins with high affinity binding (low nM) to fluorescein and digoxygenin have been engineered (Gebauer et al., 2012, Methods Enzymol 503: 157-188).

In some embodiments, the binding agent is derived from a biological, naturally occurring, non-naturally occurring, or synthetic source. In some embodiments, the binding agent is derived from de novo protein design (Huang et al., (2016) 537(7620):320-327). In some embodiments, the binding agent has a structure, sequence, and/or activity designed from first principles.

In certain embodiments, an amino acid in complex with the coupler is also contacted with a non-cognate binding agent. As used herein, a non-cognate binding agent is referring to a binding agent that is selective for a different target (e.g. different amino acid) than the particular target being considered. For example, if the amino acid in complex with the coupler is phenylalanine, and it is contacted with three binding agents selective for phenylalanine, tyrosine and asparagine in complex with the coupler, respectively, the first binding agent selective for the phenylalanine target would be a cognate binding agent, while the other two binding agents would be non-cognate binding agents for that target (since they are selective for amino acids other than phenylalanine). The tyrosine and asparagine binding agents may, however, be cognate binding agents for other targets immobilized on the support. If the phenylalanine target was then released from the support, and in the next cycle a tyrosine target is attached (the next amino acid of the polypeptide is tyrosine), and it was then contacted with the same three binding agents, the binding agent selective for tyrosine in complex with the coupler would be cognate binding agent, while the other two binding agents would be non-cognate binding agents. Thus, it should be understood that whether an agent is a cognate binding agent or a non-cognate binding agent will depend on the nature of the particular amino acid currently available for binding. Also, if multiple polypeptides are analyzed in a multiplexed reaction on a support, a binding agent for one target may be a non-cognate binding agent for another target, and vice versa. According, it should be understood that the following description concerning binding agents is applicable to any type of binding agent described herein (i.e., both cognate and non-cognate binding agents).

In certain embodiments, the concentration of the binding agents in a solution is controlled to reduce background and/or false positive results of the assay.

In some embodiments, the concentration of a binding agent can be at any suitable concentration, e.g., between about 0.01 nM and about 0.1 nM, between about 0.1 nM and about 1 nM, between about 1 nM and about 10 nM, between about 10 nM and about 100 nM, between about 100 nM and about 1000 nM, or more than about 1,000 nM.

Various binding agents capable of specific binding to a single amino acid in complex with the coupler can be utilized in the claimed methods. In some embodiments, binging agents can bind selectively to a few or several structurally similar amino acids in complex with the coupler. High affinity binging agents can be engineered by methods known in the art, since their affinity towards a particular amino acid does not now depend on neighboring amino acid residues of the polypeptide, which was the case when the amino acid was a terminal amino acid residue of the polypeptide.

In some embodiments, the coupler comprises an N-terminal modification group (NTM) that binds the N-terminal amino acid (NTAA) of the immobilized polypeptide, so after release of the coupler-amino acid complex, a free carboxyl group of the amino acid is available for recognition by the binging agent. Accordingly, in some embodiments, binding agents capable of binding to the coupler-amino acid complex comprise engineered catalytically inactive carboxypeptidases. To act as a binding agent, the catalytic residues of the carboxypeptidase can be mutated to create a catalytically inactive enzyme, which still retains its binding ability. This is exemplified with the subtilisin serine proteases comprised of a canonical Ser-His-Asp catalytic triad, in which any or all of the catalytic residues can be mutated to alanine to render the enzyme largely catalytically inactive without affecting binding Km's (disclosed in Carter P, Wells J A. Dissecting the catalytic triad of a serine protease. Nature. 1988 Apr. 7; 332(6164):564-8). Exemplar carboxypeptidases suitable for engineering include the MEROPS (Rawlings, N., et al., MEROPS: the database of proteolytic enzymes, their substrates and inhibitors. Nucleic Acids Res (2014) 42: D503-D509) family S10, M14, M20 and M32 members; IUPAC classifications include EC 3.4.16 (serine carboxypeptidases), EC 2.4.17 (metallo-carboxypeptidases), and EC 3.4.18 (cysteine carboxypeptidases). The serine carboxypeptidases, the metallocarboxypeptidases, and the cysteine carboxypeptidases can be rendered catalytically inactivate by replacing residues within the Ser-His-Asp catalytic triad, the His-Xaa-Xaa-Glu (M14) or His-Glu-X-X-His (M32) motif, or the Cys-His motif with alanine, respectively.

A number of proteinaceous binding scaffolds have been evolved in nature, including antibodies, enzymes, and structural proteins (e.g. adaptor proteins). Nucleic acids also provide mechanisms to bind peptides/proteins with high affinity and specificity (e.g. aptamers). In vitro evolution of these scaffolds affords accelerated pace for discovery of binding agents with desired affinity and specificity. Several specific examples of potential binding agent scaffolds are highlighted below, with particular emphasis on those presenting biomolecular recognition of the C-terminal carboxylate of amino acids or peptides.

In some embodiments, antibodies generated using immunogen, amino acid conjugated to adjuvant, can be used for detection of the isolated amino acid in complex with the coupler. Commercially available antibodies, Anti-Phenylalanine antibody (LSBio, #LS-C696853-200), Anti-L-Aspartate antibody (abcam, #ab9439), Anti-Glycine antibody (abcam, ab9442), Anti-valine antibody (biorbyt, #orb449633) and Anti-Tryptophan Antibody (Millipore, #AB135) can be conjugated to the coding tag on Lys residue for the encoding assay.

In some embodiments, selective binding agents can be derived from a carboxypeptidase. Carboxypeptidases are proteolytic enzymes that remove C-terminal amino acids/peptides from proteins. Two enzymatic mechanisms for carboxypeptidase activity have been identified: metalloproteases that employ zinc ions to affect catalysis and serine carboxypeptidases that employ an activated serine nucleophile (Breddam, K. Serine carboxypeptidases. A review. Carlsberg Res. Commun. (1986) 51, 83). Carboxypeptidases are a diverse enzyme family that generally demonstrate substrate sequence specificity derived from interactions between the substrate and enzyme active site. Metallo-carboxypeptidases are classified in the MEROPS peptidase database as families as M14, M15, M20, M28B, and M32 whereas serine carboxypeptidases belong to the S10, S11, S12, S28, S41, and S66. In many carboxypeptidases, C-terminal specificity (vs. aminopeptidase activity) is mediated by interactions between the C-terminal carboxylate on the substrate and an arginine in the metalloprotease. Carboxypeptidases vary in C-terminal amino acid specificity, and highly specific enzymes may represent particularly useful candidates for binding agent derivation. Importantly, catalytic activity may be removed through genetic engineering or biochemical regulation (e.g. addition of inhibitor or metal chelator).

In some embodiments, selective binding agents can be derived from the S10 family, which comprises serine proteases with yeast Carboxypeptidase Y (CPY) as an exemplar member. CPY is used in peptide sequencing since it is processive and has very little bias for amino acid residues it removes from the C-terminus of the peptide (Patterson, D., et al, C-terminal ladder sequencing via matrix-assisted laser desorption mass spectrometry coupled with carboxy-peptidase Y time-dependent and concentration-dependent digestions. Anal. Chem. (1995) 67:3971-3978 Jung G, et al., Carboxypeptidase Y: structural basis for protein sorting and catalytic triad. J Biochem. 1999 July; 126(1):1-6). The CPY binding pocket for the C-terminal amino acids (P₁′ residue) is comprised of mostly hydrophobic residues including Trp49, Asn51, Gly52, Cys56, Thr60, Phe64, Glue5, Glu145, Tyr256, Tyr269, Leu272, Ser297, Cys298 and Met398. CPY exhibits a broad specificity to the C-terminal residue of polypeptide substrates accommodating hydrophobic, hydrophilic, and aliphatic, residues due to its large binding pocket at the S₁′ site in CPY. In contrast, CPY exhibits much greater specificity for the P1 residue (penultimate to C-terminus). The S1 subsite is a deep pocket mainly constructed of hydrophobic residues, Tyr147, Leu178, Tyr185, Tyr188, Asn241, Leu245, Trp312, Ile340, and Cys341 rendering a hydrophobic preference for the C-terminal penultimate residue (disclosed in U.S. Pat. No. 5,945,329 Customized Protease; U.S. Pat. No. 5,985,627 Modified Carboxypeptidase). The C-terminal recognition of CPY is accomplished strictly by hydrogen bonding. The carboxyl terminus of the peptide forms hydrogen bonds with the backbone amide of Gly52 and the side chains of Asn5l and Glu145 in CPY. Accordingly, the coupler can be designed to mimic a hydrophobic preference for the C-terminal penultimate residue, and the large binding pocket at the S₁′ site in CPY can be engineered in multiple ways to provide selectivity for multiple isolated amino acids in complex with the coupler.

In some embodiments, selective binding agents can be derived from the M14 family, which is comprised of metallo-carboxypeptidases including Carboxypeptidase A (CPA), Carboxypeptidase B (CPB), and Carboxypeptidase T (CPT) such as the thermophilic bacterial carboxypeptidase from Thermoactinomyces vulgaris (F. Gomis-Ruth, Structure and Mechanism of Metallocarboxypeptidases, Critical Reviews in Biochemistry and Molecular Biology, (2008) 43:5, 319-345). The compact globular shape of the funnelin carboxypeptidases and cone-like entrance to the binding pocket are well suited to being engineered as C-terminal binding agents. Various members of the M14 family exhibit different C-terminus specificities. For instance, Thermoactinomyces vulgaris CPT has broad substrate specificity against hydrophobic, hydrophilic, and charged residues at the C-terminus. This contrasts with the narrow substrate specificity of the CPA and CPB families which hydrolyze hydrophobic and positively charged residues, respectively, from the C-termini of the peptides (Akparov, V., et al., Structural insights into the broad substrate specificity of carboxypeptidase T from Thermoactinomyces vulgaris. FEBS Journal, (2015), 282(7), 1214-1224; Akparov, V., et al., Structural principles of the wide substrate specificity of Thermoactinomyces vulgaris carboxypeptidase T. reconstruction of the carboxypeptidase B primary specificity pocket. Biochemistry. Biokhimiia, (2007), 72(4), 416-423).

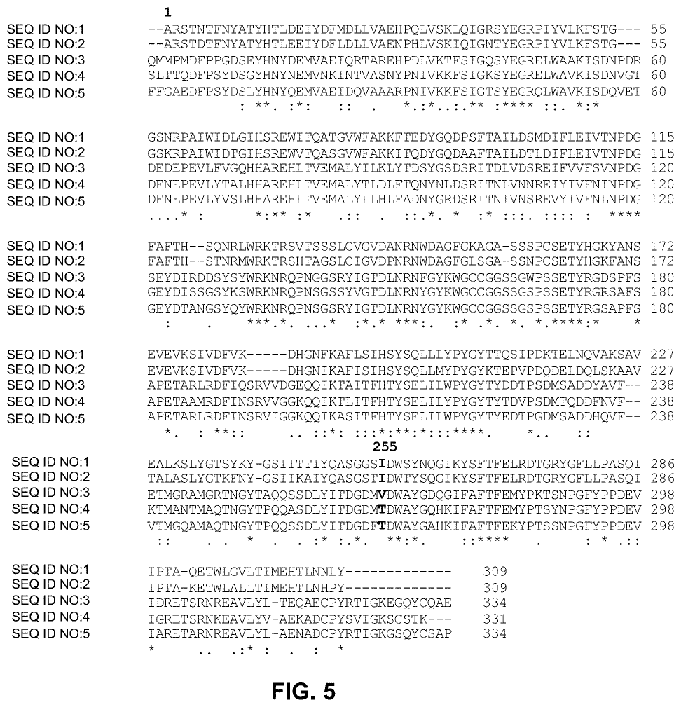

The specificity for the C-terminal residue is largely determined by the identity of the amino acids comprising the specificity or binding pocket. As such, the M14 funnelin family of carboxypeptidases can have their specificity/binding pocket altered through directed evolution by mutating residues in the specificity/binding pocket. In particular, residues (CPA numbering) at locations 194, 203, 207, 243, 247, 248, 250, 253-255, and 268 play critical roles in C-terminal amino acid specificity (Gomis-Ruth, 2008). This is exemplified by the specificity conferred by residue 255 in which isoleucine is present in CPA (hydrophobic residue preference), aspartate in CPB (positively charged residue preference), threonine in CPT (broad specificity), and arginine in M14 N/E-type carboxypeptidases (negatively charged residue specificity) (Akparov, 2007), see FIG. 5. Other residues such as Arg145, Tyr248, and Asn144 involved in binding to the C-terminal carboxylate should remain unaltered since they are involved in formation of a salt bridge with the C-terminal-carboxylate of the substrate. In a preferred embodiment, a thermophilic CPT such as P. halophilum, Th. vulgaris, or L. thermophila is used as a scaffold for binding agent.

Human leukocyte antigens (HLA's) in humans (aka major histocompatibility complex (MHC) in animals) are part of a naturally evolved mechanism to derive antibodies against a particular (protein derived) antigen. Upon intracellular digestion of a foreign protein, resulting peptides are bound to HLA molecules and displayed on the extracellular surface for recognition by host T-lymphocytes. Significant binding energy is derived from interactions with the C-terminal region of the peptide (Guillaume P, et al., Proceedings of the National Academy of Sciences, 2018, 115 (20) 5083-5088), indicating these proteins may provide useful scaffolds to derive C-terminal binding agents. While N-terminal degrons were the first identified, a number of C-terminal degrons have been identified recently (Guillaume P, et al., Proceedings of the National Academy of Sciences, 2018, 115 (20) 5083-5088). E3 ubiquitin ligases of the cullin-RING (CRL) family recognize C-terminal amino acids or motifs to facilitate targeted protein degradation (Varshavsky A. N-degron and C-degron pathways of protein degradation. Proc Natl Acad Sci USA. 2019; 116(2):358-366). C-degron pathways for glycine, arginine, aspartate, alanine, and valine have identified and these proteins are therefore potential candidates to derive tethered amino acid binding agent scaffolds (Timms and Koren, “Tying up loose ends: the N-degron and C-degron pathways of protein degradation”. Biochem Soc Trans 28 Aug. 2020; 48 (4): 1557-1567).

In some embodiments, a binding agent configured to bind selectively to the tethered coupler-amino acid complex comprises an amino acid sequence having at least 60%, at least 70%, at least 80%, at least 90%, or at least 95% sequence identity to any one of the following amino acid sequences: SEQ ID NO: 1-SEQ ID NO: 5. The sequences listed are the active carboxypeptidase enzymes of about 35 kDa in size. The active enzyme is derived from the pre-pro-carboxypeptidase sequences. Typically a 13 amino acid signal sequence is cleaved upon secretion, and a 95 amino acid propeptide is cleaved to activate the carboxypeptidase (Clauser E. et al., J. Biol. Chem. 1988, Vol. 263, 17837-45).

In some embodiments, binding agents capable of binding to the coupler-amino acid complex are engineered from aminoacyl tRNA synthetases (aaRSs). The aaRSs are a class of proteins with exquisite amino acid binding specificity. The set of 20+ aaRSs exhibit various modes of binding to the amino acids (AAs) including hydrophobic binding, hydrogen binding, salt-bridges, and pi-pi stacking (Kaiser F, et al. The structural basis of the genetic code: amino acid recognition by aminoacyl-tRNA synthetases. Sci Rep (2020) 10, 12647). In fact, aaRSs have been suggested as tools for single molecule protein sequencing (U.S. Pat. No. 9,435,810 B2; Borgo, B, “Strategies for Computational Protein Design with Application to the Development of a Biomolecular Tool-kit for Single Molecule Protein Sequencing” (2014). All Theses and Dissertations (ETDs). 1221; Sampath G., “Single Molecule Protein Sequencing Based on the Superspecificity of tRNA Synthetases”. Preprints.org; 2020). These references describe using engineered aaRSs as N-terminal binding agents, but engineered aaRSs also offer the capability of generating high affinity interactions with amino acids having an exposed C-terminus. The aaRSs activate the cognate amino acids through C-terminal conjugation of ATP to produce an aminoacyl adenylate intermediate, which involves significant binding energy generation due to hydrophobic and hydrogen bonding. The activated amino acid is subsequently transferred to a respective tRNA to “charge” the tRNA for subsequent protein synthesis. As such, adenylation of the C-terminal amino acid on the coupler-amino acid complex during aaRS binding and subsequent adenylation should lead to greatly increased affinity (Carter C W Jr, et al. “The Rodin-Ohno hypothesis that two enzyme superfamilies descended from one ancestral gene: an unlikely scenario for the origins of translation that will not be dismissed”. Biol Direct. 2014; 9:11). In one embodiment, an engineered catalytic portion of aaRS is used for coupler-amino acid complex binding. In another embodiment, an engineered enzyme portion of the aaRS is used for coupler-amino acid complex binding (Pham, Yen et al., Tryptophanyl-tRNA Synthetase Enzyme, Journal of Biological Chemistry, Volume 285, Issue 49, 38590-38601).

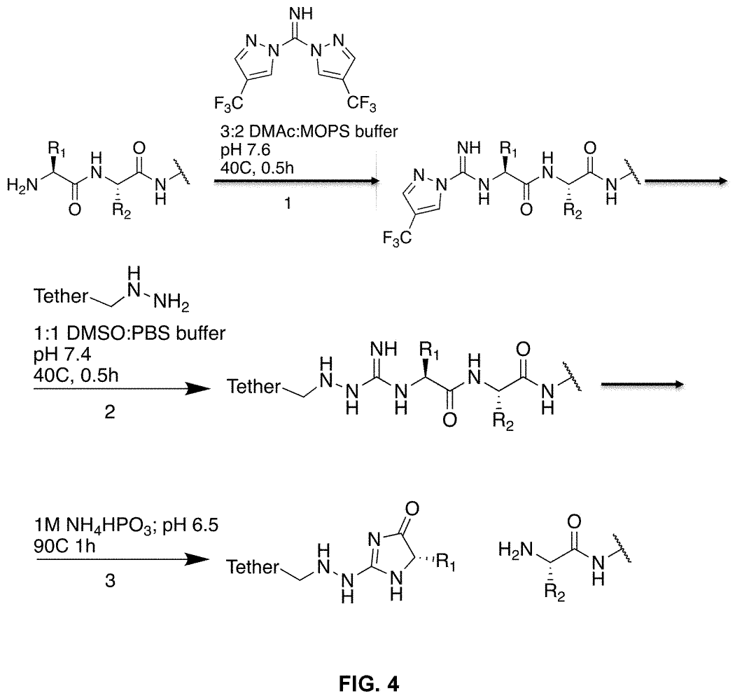

To obtain at least partially selective binding agents, portions of the coupler-amino acid complexes can be used as antigens for the development of antibodies with high affinity and specificity. In one method, the coupler-amino acid complexes can be injected into rabbits (optionally conjugated with Keyhole limpet haemocyanin antigen) to elicit an immune response and production of antibodies against the complexes. Exemplary portions of the coupler-amino acid complexes are shown in FIG. 4 (as a product of step 3 attached to the tether moiety). Monoclonal antibodies generated via rabbit hybridoma technology can be tested for affinity, specificity and cross-reactivity. The antibodies secreted by the different clones can be assayed for cross-reactivity using enzyme-linked immunosorbent assay and affinity of the antibodies can be measured using the label-free method BioLayer Interferometry.

If produced antibodies do not display robust affinity or specificity towards coupler-amino acid complexes, directed evolution approaches can be used to improve antibody affinity or specificity. Standard, well known screening techniques can be utilized, including phage display or yeast display. For example, clones generated from rabbit hybridoma can be used to construct an antibody library in yeast. Yeast display has been used to successfully engineer antibodies that target small molecules with high affinity by directed evolution via mutagenesis. Negative selection can be utilized in yeast display to remove antibodies that cross-react with other complexes. Negative selection would involve incubating yeast cells expressing the antibody library with beads conjugated to non-target antigens and pulling them out of solution. When targeting antibodies specific for a particular coupler-amino acid complex, the other 19 amino acids in complex with the coupler can be negatively selected against to improve the probability of generating a specific binding agent.

Methods to select binding agents that specifically recognize modified NTAA residues of immobilized peptides have been disclosed (see, e.g., U.S. patent application Ser. Nos. 17/539,033 and 17/727,677, incorporated herein). Similar techniques can be applied to select binding agents specific for particular coupler-amino acid complexes (see also Examples 6 and 7).

### Coupler

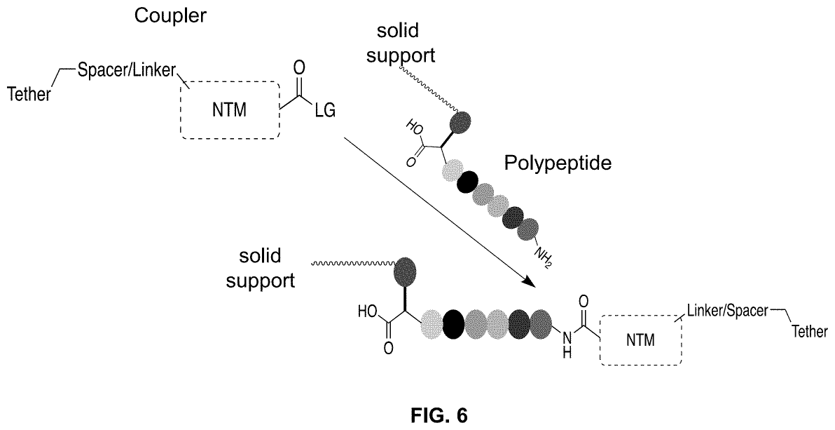

In some embodiments, the method comprises treating the immobilized polypeptide with a coupler that modifies a terminal amino acid of the polypeptide. In preferred embodiments, the coupler amenable to proximity tethering comprises a N-terminal modification group (NTM group) that reacts with the N-terminal amino acid (NTAA) of the immobilized polypeptide, and also comprises a pre-installed linker or bioorthogonal handle (see, for example, FIG. 6). For reaction between the NTM group and the polypeptide, NTM groups in the form of active esters can be dissolved in one of the following polar organic solvents; acetonitrile (ACN), N,N-dimethylformamide (DMF), N,N-dimethylacetamide (DMAc), N-methyl-2-pyrrolidone (NMP), sulfolane, dimethylsulfoxide (DMSO), cyrene, 1,3-dimethyl-2-imidazolidinone (DMI), and 1,3-Dimethyl-3,4,5,6-tetrahydro-2(1H)-pyrimidinone (DMPU). Buffers used for this reaction are typically selected from: sodium acetate, potassium acetate, ammonium acetate, sodium phosphate, potassium phosphate, ammonium phosphate, PBS, MES, MOPS, HEPES, Tris-HCl, NEMA, PIPES, HEPPSO, triethylammonium acetate, triethanolammonium acetate, citrate, cit-phos, CAPS, CAPSO, bicarbonate, carbonate-bicarbonate, carbonate, borate, and bis-tris, where the pH of the buffer is in a range of 4-12; typically 6-11, and preferably 7-10. In preferred embodiments, to affix a NTM group (examples of shown by Formulas 1-3 below) onto the N-terminus of a solid surface-attached polypeptide, an active ester is formed from a carboxylic acid and a leaving group, the reagent is dissolved in a polar organic solvent or mixture thereof in 1-200 mM concentration with some percentage of aqueous buffer (1-80%). The solution is then added to the polypeptide and allow to react for 1-180 minutes at a temperature 4-60° C. Upon completion of the reaction, the solid surface is washed with 70% ethanol and PBS-T buffer to remove excess reagents.

In some embodiments, the coupler comprises a carbo- or hetero-cyclic ring containing various positional substituents including a linker or spacer, an optional natural or unnatural amino acid, and a tethering or attachment moiety.

In some embodiments, the unnatural amino acid is comprised of one of the following (Lee K J, et al., Site-Specific Labeling of Proteins Using Unnatural Amino Acids. Mol Cells. 2019 May; 42(5) 386-396): Nε-p-azidobenzyloxycarbonyl lysine (PABK), Propargyl-L-lysine (PrK), Nε-(1-methylcycloprop-2-enecar-boxamido)lysine (CpK), Nε-acryllysine (AcrK), Nε-(cyclooct-2-yn-1-yloxy)carbonyl)L-lysine (CoK), bicyclo[6.1.0]non-4-yn-9-ylmethanol lysine (BCNK), trans-cyclooct-2-ene lysine (2′-TCOK), trans-cyclooct-4-ene lysine (4′-TCOK), dioxo-TCO lysine (DOTCOK), 3-(2-cyclobutene-1-yl)propanoic acid (CbK), NF-5-norbornene-2-yloxycarbonyl-L-lysine (NBOK), cyclooctyne lysine (SCOK), 5-norbornen-2-ol tyrosine (NOR), cyclooct-2-ynol tyrosine (COY), (E)-2-(cyclooct-4-en-1-yloxyl)ethanol tyrosine (DS1/2), 19: azidohomoalanine (AHA), homopropargylglycine (HPG), azidonorleucine (ANL), and NF-2-azideoethyloxycarbonyl-L-lysine (NEAK).

In some embodiments, the NTAA of the polypeptide is contacted with the NTM group of the coupler under conditions that allow the NTAA to conjugate to the primary amine reactive moiety of the coupler to form a coupler-polypeptide complex.

In preferred embodiments, upon binding the coupler forms a covalent linkage with the terminal amino acid of the polypeptide or with the modified terminal amino acid of the polypeptide forming the coupler-polypeptide complex.

In some embodiments, the coupler has a structure encompassed by one of the following Formulas (1-3):

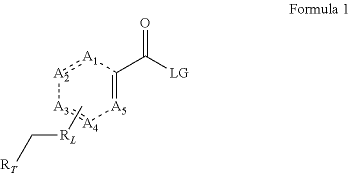

wherein: LG is a leaving group selected from halo, N-hydroxysuccinimide (NHS), N-hydroxybenzotriazole, sulfo N-hydroxysuccinimide (sulfoNHS), 2,3,4,5,6-pentafluorophenol (PFP), 4-sulfo-2,3,5,6-tetrafluoro phenol, chloro, 4-nitrophenol, hexafluoroisopropanol and —O(C═)—O—(C1-C6 alkyl);

A₁-A₅ are each independently selected from CH, CX, and N;
X at each occurrence is independently selected from H, C₁-C₂ alkyl, NO₂, C₁-C₂ haloalkyl, C₁-C₂ haloalkoxy, halo, —OR₂, —N(R₂)₂, —SR₂, SO₂R₃, SO₃R₂, —B(OR₂)₂, C(═O)R₂, CN, CON(R₂)₂, —COOR₂, —C(—O)Ar, and tetrazole;
RL linkers at each occurrence is independently selected from
Polyethylene glycol (PEGₙ), where n=1-24 (number of PEG molecule joined together);
Beta-alanine repeated units, where number of repeats is from 1 to 24;
6-aminohexanoic acid repeat units, where number of repeats is from 1 to 12;
or independently selected from the following:

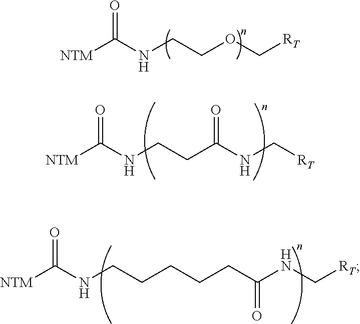

RT at each occurrence is independently selected from biotin, desthiobiotin, 4-chlorobenzamide, 4-sulfamoylbenzamide, 3-sulfamoyl-4-chlorobenzamide, or another protein/enzyme ligand. In another embodiment, RT can be a chemically reactive moiety such a click chemistry moiety including iEDDA moieties such as mTET or TCO.

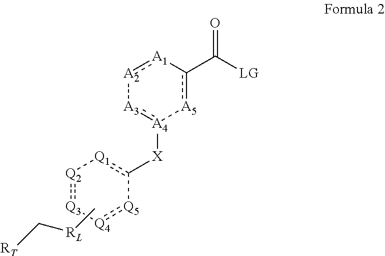

wherein:

LG is a leaving group selected from halo, N-hydroxysuccinimide (NHS), N-hydroxybenzotriazole, sulfo N-hydroxysuccinimide (sulfoNHS), 2,3,4,5,6-pentafluorophenol (PFP), 4-sulfo-2,3,5,6-tetrafluoro phenol, chloro, 4-nitrophenol, hexafluoroisopropanol and —O(C═)—O—(C1-6 alkyl);
A₁-A₅ are each independently selected from CH, CY, and N;
Y at each occurrence is independently selected from H, C₁-C₂ alkyl, NO₂, C₁-C₂ haloalkyl, C₁-C₂ haloalkoxy, halo, —OR₂, —N(R₂)₂, —SR₂, SO₂, SO₃, —B(OH)₂, C(═O), CN, CON, —COOR₂, —C(—O)Ar, and tetrazole;
X at each occurrence is independently selected from C1-C2 alkyl, CH2OCH2-, NH—, CONH—, C(═O)—, CON—, SO2-, NHSO2-, 1,2,3-triazole-;
RL linkers at each occurrence is independently selected from
Polyethylene glycol (PEGₙ), where n=1-24 (number of PEG molecule joined together);
Beta-alanine repeated units, where number of repeats is from 1 to 24;
6-aminohexanoic acid repeat units, where number of repeats is from 1 to 12;
or independently selected from the following:

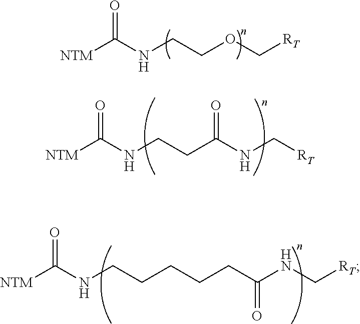

each of Q₁-Q₅ is individually selected from CH, CZ, and N;
Z at each occurrence is individually selected from H, C₁-C₂ alkyl, NO₂, C₁-C₂ haloalkyl, C₁-C₂ haloalkoxy, halo, —OH, —OCH₃, —OCF₃, —OCF₂H, —N(CH₃)₂, —SCH₃, SO₂NH₂, SO₃, —B(OH)₂, C(═O)CH₃, CN, CONH₂, —COOH, NO₂, tetrazole;
RT at each occurrence is independently selected from biotin, desthiobiotin, 4-chlorobenzamide, 4-sulfamoylbenzamide, 3-sulfamoyl-4-chlorobenzamide, or another protein/enzyme ligand. In another embodiment, RT can be a chemically reactive moiety such a click chemistry moiety including iEDDA moieties such as mTET or TCO.

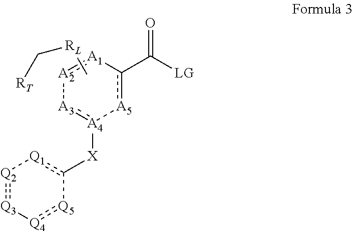

wherein:
LG is a leaving group selected from halo, N-hydroxysuccinimide (NHS), N-hydroxybenzotriazole, sulfo N-hydroxysuccinimide (sulfoNHS), 2,3,4,5,6-pentafluorophenol (PFP), 4-sulfo-2,3,5,6-tetrafluoro phenol, chloro, 4-nitrophenol, hexafluoroisopropanol and —O(C═)—O—(C1-6 alkyl);
A₁-A₅ are each independently selected from CH, CY, and N;
Y at each occurrence is independently selected from H, C₁-C₂ alkyl, NO₂, C₁-C₂ haloalkyl, C₁-C₂ haloalkoxy, halo, —OR₂, —N(R₂)₂, —SR₂, SO₂, SO₃, —B(OH)₂, C(═O), CN, CON, —COOR₂, —C(—O)Ar, and tetrazole;
X at each occurrence is independently selected from C₁-C₂ alkyl, CH₂OCH₂—, NH—, CONH—, C(═O)—, CON—, SO₂—, NHSO₂—, 1,2,3-triazole-;
RL linkers at each occurrence is independently selected from
Polyethylene glycol (PEGn), where n=1-24 (number of PEG molecule joined together);
Beta-alanine repeated units, where number of repeats is from 1 to 24;
6-aminohexanoic acid repeat units, where number of repeats is from 1 to 12;
or independently selected from the following:

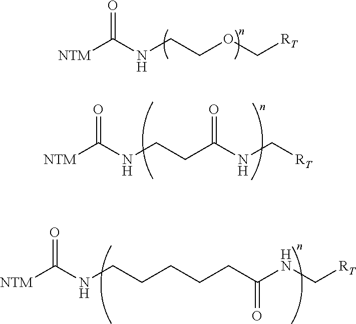

each of Q₁-Q₅ is individually selected from CH, CZ, and N;

Z at each occurrence is individually selected from H, C₁-C₂ alkyl, NO₂, C₁-C₂ haloalkyl, C₁-C₂ haloalkoxy, halo, —OH, —OCH₃, —OCF₃, —OCF₂H, —N(CH₃)₂, —SCH₃, SO₂NH₂, SO₃, —B(OH)₂, C(═O)CH₃, CN, CONH₂, —COOH, NO₂, tetrazole;
RT at each occurrence is independently selected from biotin, desthiobiotin, 4-chlorobenzamide, 4-sulfamoylbenzamide, 3-sulfamoyl-4-chlorobenzamide, or another protein/enzyme ligand. In another embodiment, RT can be a chemically reactive moiety such a click chemistry moiety including iEDDA moieties such as mTET or TCO.

In some embodiments, the coupler comprises an N-terminal modification group (NTM) that comprises one of the following moieties: N-hydroxysuccinimide esters (NHS esters), isothiocyanate, tetrabutylammonium isothiocyanate, diphenylphosphoryl isothiocyanate, phenyl isothiocyanate (PITC), glyoxals, oxiranes, isocyanates, acyl azides, sulfonyl chlorides, aldehydes, carbonates, epoxides, aryl halides, imidoesters, carbodiimides, anhydrides, and fluorophenyl esters.

In a preferred embodiment, the N-terminal amino acid (NTAA) 4 of the DNA-polypeptide conjugate is functionalized with a coupler comprising a NTM group 8 (for interaction with the NTAA), a tether, and a second stabilizing component 7 (FIG. 1B and FIG. 2B). Before, together with or after the coupler binding to the NTAA, a first stabilizing component 6 is associated with the recording tag 1. In a preferred embodiment, the first stabilizing component 1 is reversibly associated with a capture DNA 2 comprising the recording tag 1 by hybridization of a complementary DNA with the first stabilizing component 1 to a cognate region on the capture DNA 2 (FIG. 2C). After tethering, cleavage, and binding/encoding of the tethered single amino acid (AA) complex with the coupler, the complex can be removed by dehybridization via heat or use of denaturing reagents such as NaOH (e.g. 0.1 NaOH/0.1% Tween-20), formamide (e.g. 90-100%), urea (e.g. 6M), and so forth. After washing with PBS-T buffer, the immobilized conjugate is now ready for the next cycle of the assay that will analyze newly exposed terminal amino acid.

In some embodiments, the N-terminal modification of the immobilized polypeptide occurs before contacting the polypeptide with the coupler. In this embodiment of the claimed methods, the terminal amino acid is modified to produce a modified terminal amino acid before contacting the polypeptide with the coupler; during contacting the coupler binds to the modified terminal amino acid; after cleaving the peptide bond between the terminal amino acid and the penultimate terminal amino acid of the polypeptide, a coupler-modified amino acid complex attached to the solid support is generated; and the binding agent is used that is capable of binding to the coupler-modified amino acid complex. In some embodiments of this embodiment, the N-terminal modification occurs by contacting the immobilized polypeptide with one of the NTM structures from Formulas 1-3 above, additionally comprising a reactive handle; and the coupler has a complementary reactive handle that reacts specifically with the reactive handle, and additionally comprises a tethering group or a stabilizing component for attachment to a solid support. In some embodiments, the N-terminal modification of the immobilized polypeptide is produced enzymatically by a natural or engineered enzyme.

In a preferred embodiment, after the stabilizing components are attached to the capture DNA and to the polypeptide through the coupler, the immobilized DNA-polypeptide conjugate is incubated with a linking agent that crosslinks the two stabilizing components forming a tethering complex. The polypeptide together with the coupler, linking agent and two stabilizing components are forming a “bridge” structure, where both termini of the polypeptide are tethered or attached indirectly to the solid support. The NTAA of the polypeptide modified by the coupler is then cleaved. In a preferred embodiment, the NTAA is cleaved with a engineered enzyme such as modified dipeptidyl peptidase enzyme that cleaves at the peptide bond between the NTAA and the penultimate amino acid. The cleaved NTAA is now tethered to capture DNA associated with the recording tag and is accessible to interaction with binding agents and subsequent encoding. Given that the alpha amino group of the NTAA is modified with the coupler, the carboxyl terminus of the NTAA is exposed for binding. In a preferred embodiment, the binding agent makes contact with both the tethered NTAA and the NTM group of the coupler. This format provides greater surface area and ostensibly higher binding affinity for the binding agent. In a preferred embodiment, the tethering complex is stable enough during transfer of the information regarding the binding agent; the tethering complex is released from the surface or disassembled after the transfer of the information regarding the binding agent is complete. In this embodiment, the tethering complex should be stable for the whole period of time that is required for the information transfer, which can be in the range of a few minutes to several hours depending on the means used for the information transfer, such as primer extension reaction, a chemical ligation or a biological ligation. At the same time, in this embodiment, the formation of the tethering complex should be controllable and reversible. After the transfer of the information, the tethering complex can be released from the solid support or disassembled via a number of ways. For example, the tethering complex can be disrupted by introducing a destabilizing agent, such as heat, a denaturing agent, an enzyme, a competitor molecule, or a combination thereof. In another example, the first stabilizing component is releasably associated with the polypeptide attached to the solid support, for example, by comprising a polynucleotide that hybridizes to a capture DNA associated with the polypeptide and the recording tag. In this example, the first stabilizing component has two or more components; one component is the polynucleotide used for hybridization with the capture DNA, and another component is a moiety that interacts with a stabilizing component or with the linking agent. In yet another example, the first stabilizing component has a lower affinity to the linking agent in comparison to an affinity of the second stabilizing component to the linking agent, which can be utilized during disassembly of the tethering complex and elution of its components (see Example 1). In some embodiments, the claimed methods further comprise a washing step to remove the released coupler-amino acid complex.

In some embodiments, the first stabilizing component can be associated with the polypeptide either before or after contacting the polypeptide with the coupler. In this embodiment, the first stabilizing component can be releasably associated with the polypeptide attached to the solid support, for example, by comprising a polynucleotide that hybridizes to a capture DNA associated with the polypeptide and the recording tag (FIG. 2C).

In some embodiments of the claimed methods, the linking agent activates one of the stabilizing components. In one example, the linking agent could act to activate the second stabilizing component by deblocking it. For instance, the second stabilizing component could be a “blocked” tetrazine which is either photoactivated or chemically activated by addition of the linking agent. After activation, the second stabilizing component is capable of reacting specifically with the first stabilizing component, forming a tethering complex. In another example, linking agent can activate blocked desthiobiotin or biotin, which then can react with the corresponding partner (e.g. avidin), acting as the first stabilizing component.

In some embodiments of the claimed methods, the polypeptide is obtained by fragmenting proteins from a biological sample.

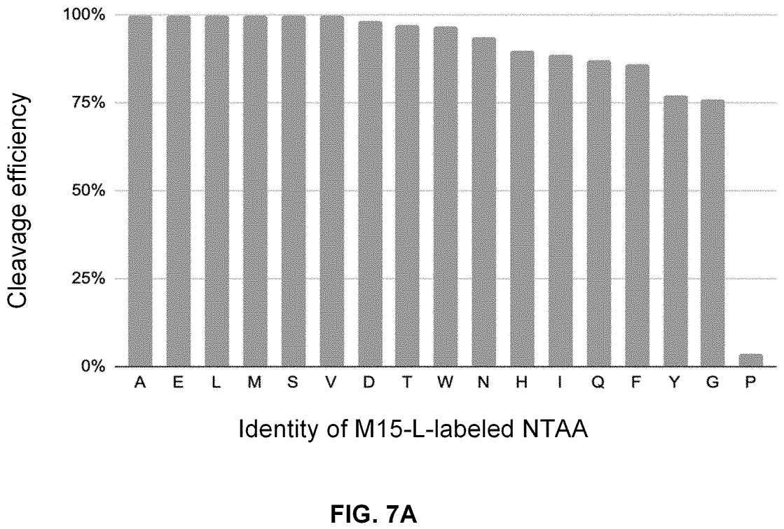

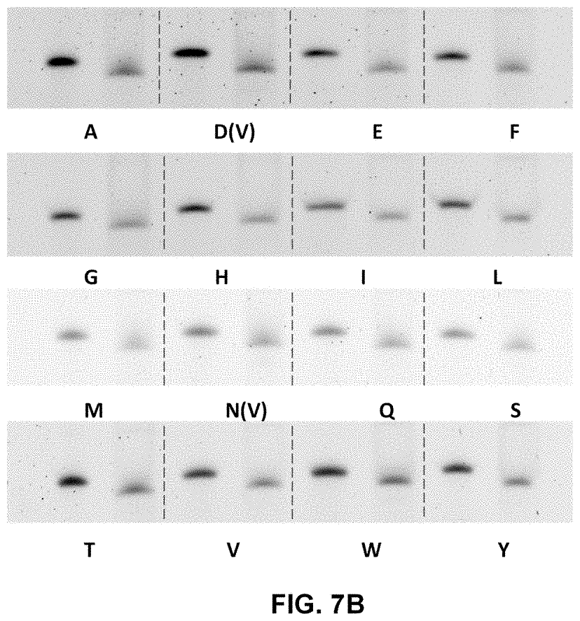

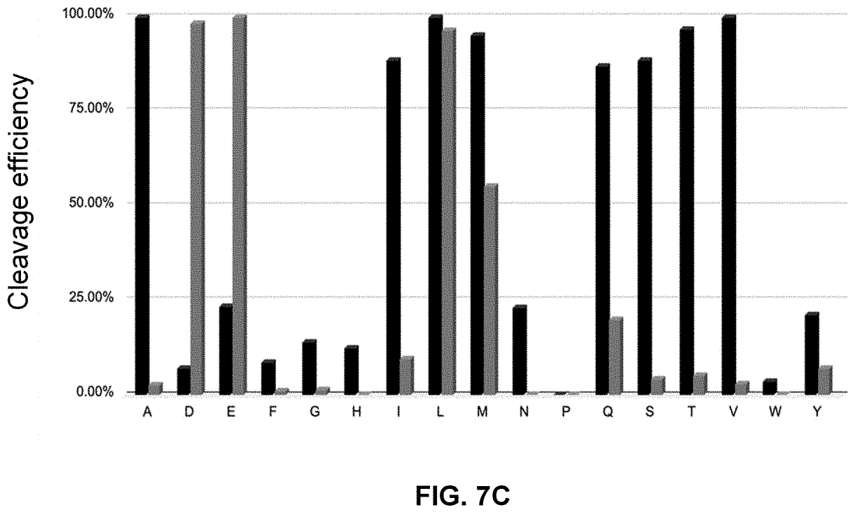

In some embodiments, the coupler-amino acid complex is cleaved from the polypeptide by a modified dipeptidyl peptidase comprising an unmodified dipeptidyl peptidase comprising at least one mutation in a substrate binding site, wherein (i) the unmodified dipeptidyl peptidase removes two terminal amino acids from a polypeptide; and (ii) the modified dipeptidyl peptidase is configured to cleave the coupler-amino acid complex from the polypeptide. Dipeptidyl peptidases can be evolved by introducing mutations in the substrate binding site to recognize and cleave the NTAA in complex with the coupler instead of a native terminal dipeptide from the polypeptide. Particular examples of such dipeptidyl peptidases together with methods of making such dipeptidyl peptidases were disclosed by the inventors in U.S. application Ser. No. 17/213,169, WO 2020/198264 and international application PCT/US2021/023347. In particular, feasibility of evolving DAP BII enzyme to cleave N-terminal modified amino acids have been demonstrated. The evolved cleavase is capable of cleaving peptide substrates that are anchored by the C-terminus to a solid support through a long PEG linker. The S2 pockets of dipeptidyl peptidases were shown to be tolerant to a variety of different amino acid sidechains, which is not surprising since dipeptidyl peptidases rely on S1 pocket for substrate specificity. The newly evolved substrate pocket for N-terminal modification also showed tolerance to alteration around the starting modification, showing plasticity to modifications (FIG. 7A-FIG. 7C). Based on these observations, random mutations can introduced around of the substrate binding site of the DAP BII enzyme in areas that are involved in recognition of the coupler moiety (“mod pocket”) and the terminal amino acid (“S2 pocket”) to create tolerance for tethered substrates.

In some embodiments, modified dipeptidyl peptidase configured to cleave the peptide bond between the terminal amino acid and the penultimate terminal amino acid of the immobilized polypeptide within the coupler-polypeptide complex comprises an amino acid sequence having at least 30%, at least 40%, at least 50%, at least 60%, at least 70%, at least 80%, or at least 90% sequence identity to the amino acid sequence of SEQ ID NO: 6. In some embodiments, modified dipeptidyl peptidase comprises an amino acid sequence having at least 30%, at least 40%, at least 50%, at least 60%, at least 70%, at least 80%, or at least 90% sequence identity to the amino acid sequence of SEQ ID NO: 6, and further comprises one or more amino acid modifications in residues corresponding to positions 214, 215, 219, 329, 673 of SEQ ID NO: 6.

In some embodiments of the method, exemplary stabilizing components comprise both covalent and non-covalent interactions with the respective linking agents. Examples of pairs of stabilizing components and linking agents comprise: streptavidin (and variants such as avidin, avidin homologues, neutravidin, traptavidin, etc.) and biotin (and variants such as iminobiotin, desthiobiotin, and so on); covalent coupling pairs comprised of nucleophilic and electrophilic reactive moieties (for example, listed in Hermanson G, Bioconjugate Techniques, (2013) Academic Press) and moieties that can interact with each other through bioorthogonal click chemistry pairs including azide-alkyne (CUAAC and SPAAC pairs), iEDDA pairs including tetrazine derivatives and dienophiles such as strained trans-cyclooctene (TCOs), norbornenes, cyclootynes, cyclopropenes (Oliveira et al. Inverse electron demand Diels-Alder reactions in chemical biology. Chem. Soc. Rev., 2017, 46, 4895-4950). Bioorthogonal reaction pairs react with a high yield and high specificity suitable for isolating a target molecule from complex biological mixtures; in particular they would not react with other components of the system or with other biomolecules.

In the preferred embodiment, the linking agent is multifunctional. Exemplar chemical linking agents include both homobifunctional (e.g. bind to the same stabilizing components) and heterobifunctional (e.g. bind to different stabilizing components) linking agents that comprise reactive moieties flanking an aliphatic or a polyethylene glycol (PEG)-based linker (such as based on PEG4, or tetraethyleneglycol). Commercially useful linkers include TCO-PEG11-TCO (Conju-Probe), San Diego, Calif.) used to link mTet stabilizing components, with one mTet attached to a DNA probe hybridized to the capture DNA comprising the recording tag, and the other mTet molecule incorporated in the coupler attached to the NTAA of the polypeptide (see Example 2 below). Other homobifunctional (e.g. bind to the same stabilizing components) and heterobifunctional (e.g. bind to different stabilizing components) linking agents comprise streptavidin and its variants such as avidin, avidin homologues, neutravidin, traptavidin, which can bind two ligands (either identical or different). In some embodiments, the iEDDA moieties are rendered inactive via photocaging or chemical caging, and activated on demand via light or chemical decaging, respectively (Li et al. Photo-controllable bioorthogonal chemistry for spatiotemporal control of bio-targets in living systems Chem. Sci., 2020, 11, 3390). The use of “on-demand” decaging of tetrazines enables activation of tetrazine after the N-terminal modification and direct coupling to a TCO stabilizing component associated with the recording tag-polypeptide conjugate; the use of a linking agent is not needed in this case. If the TCO is associated to the recording tag-polypeptide conjugate via hybridization, the tethering complex can be easily removed by heat or denaturation.

In some embodiments of the method, exemplary components of the tethering complex formed by stabilizing components and a linking agent comprise engineered macrocyclic receptors configured to bind their corresponding ligands. These engineered macrocyclic receptors include host calixarenes, cucurbiturils, cyclodextrins, and pillararenes with two independent guests. In one example, cucurbituril CB[8] rotaxane-based complexes can be formed between a first guest attached to 5′ end of capture DNA comprising the recording tag and a second guest attached to the coupler. The exemplary first and second guests include: one electron-poor guest (e.g. methyl viologen, MV) and one electron rich guest (e.g. Naphthyl, Na). The tethering complex is formed between the two guests, MV, Na, and CB[8] lead to a high-affinity complex with a dissociation constant of 5×10¹¹ M⁻² (Gurbuz, S., et al., Cucurbituril-based supramolecular engineered nanostructured materials, (2015), Org Biomol Chem 13(2): 330-347). This CD[8] complex can be dissociated, on demand, by incubating with mM concentrations of the competitive binding compound, 1-adamantylamine (ADA), which has an affinity constant of 10¹². Alternatively, 2,6-dihydroxynaphthalene (DHN) can be used. Additional chemical structures of guests that can be used with corresponding hosts are disclosed in Rauwald et al., Correlating Solution Binding and ESI-MS Stabilities by Incorporating Solvation Effects in a Confined Cucurbit[8]uril System, The Journal of Physical Chemistry, 2010 114 (26), 8606-8615 and in Martins et al., Selective Recognition of Amino Acids and Peptides by Small Supramolecular Receptors”, Molecules, 2021, 26, no. 1: 106.

In some embodiments, the linking agent comprises an oligonucleotide comprising a sequence complementary to a nucleic acid joined to the binding agent. In this aspect, the linking agent is also hybridized to the stabilizing component of the recording tag. In some embodiments, information is transferred from the coding tag to the linking agent, and this information is subsequently transferred to the recording tag.

In some embodiments, the polypeptide sequencing assay comprises the steps of:

(a) providing a polypeptide and an associated recording tag attached to a solid support;
(b) contacting the polypeptide with a coupler, wherein the coupler binds to a terminal amino acid of the polypeptide to form a coupler-polypeptide complex;
(c) attaching the coupler-polypeptide complex to the solid support;
(d) cleaving the coupler-polypeptide complex from the polypeptide, thereby exposing a new terminal amino acid of the polypeptide and providing a coupler-amino acid complex attached to the solid support;
(e) contacting the coupler-amino acid complex with a binding agent capable of binding to the coupler-amino acid complex, wherein the binding agent comprises a coding tag with identifying information regarding the binding agent;
(f) transferring the information of the coding tag of the binding agent to the recording tag;
(g) releasing the coupler-amino acid complex from the solid support;
(h) repeating steps (b) through (g) at least one more time;
(i) analyzing the recording tag extended after information transfer, thereby identifying at least a portion of the sequence of the polypeptide.

In some embodiments, the attaching step (c) occurs via formation of a tethering complex. In some embodiments, to perform this attachment, at step (a) the polypeptide is further associated with a first stabilizing component; at step (b) the coupler is linked to a second stabilizing component; at step (c) after binding of the coupler to the terminal amino acid of the polypeptide, the first and second stabilizing components are releasably linked together to form a tethering complex between the first stabilizing component attached to the solid support and the second stabilizing component linked to the coupler-polypeptide complex; and at step (d) the coupler-amino acid complex is releasably attached to the solid support via the tethering complex.

In some embodiments, the method comprises transferring information of a coding tag associated with the binding agent to the recording tag associated with the target polypeptide to generate an extended recording tag, thereby generating an extended recording tag. In some cases, transferring information of the coding tag to the recording tag is performed after the stabilizing components are linked and the tethering complex is formed. Methods of transferring information of a coding tag associated with the binding agent to the recording tag are disclosed in applications US 20190145982 A1, US 20200348308 A1, US 20200348307 A1, the content of which are incorporated herein.

In some embodiments, the method comprises one or more cycles, wherein in each cycle a terminal amino acid of a polypeptide immobilized on a solid support is contacted with a coupler, cleaved off the polypeptide to reveal a new terminal amino acid of the polypeptide and attached to the solid support in an isolated form available for interaction with the binding agent, which results in transferring information of the binding agent's associated coding tag to a recording tag of the polypeptide. Each cycle extends the recording tag, accumulating information regarding the binding agent that recognized the current terminal amino acid of the polypeptide. Before beginning of the next cycle, the isolated amino acid in complex with the coupler is either released or blocked to prevent further interaction with binding agents of the following cycles. On the next cycle, the next terminal amino acid of the polypeptide is contacted with the coupler, cleaved off the polypeptide and contacted in complex with the coupler with a new binding agent, followed by transferring information of the new binding agent's associated coding tag to the recording tag, further extending the recording tag associated with the polypeptide. Accordingly, in each cycle the current terminal amino acid is cleaved off the polypeptide, exposing a new terminal amino acid of the polypeptide for the next cycle. After completion of the n^{th} cycle (where n is a natural number), the recording tag is extended n times in each cycle of binding; in each cycle the transferred extension contains information regarding the binding agent bound to the current terminal amino acid of the polypeptide, and thus information regarding the terminal amino acid itself. The order of the recording tag extensions matches the order of amino acids in the polypeptide. After completion of the n^{th} cycle, the recording tag is analyzed and recovered information is used to identify at least a portion of polypeptide's sequence. In preferred embodiments, multiple polypeptides are analyzed in parallel, and the recording tags associated with these polypeptides and extended after completion of the n^{th} cycle are also analyzed in parallel, for example by next generation sequencing. Sequences of the extended recording tags are decoded to provide information regarding amino acid sequences of the polypeptides. In certain embodiments, multiple binding agents are used at the same time in parallel. This parallel approach saves time and reduces non-specific binding by non-cognate binding agents to a site that is bound by a cognate binding agent (because the binding agents are in competition). In some embodiments, C terminal amino acids (CTAAs) or N terminal amino acids (CTAAs) can be analyzed by the methods described herein. To obtain N-terminal amino acid sequences of polypeptides, the coupler is used that can recognize and bind to N-terminal amino acids of polypeptides. To obtain C-terminal amino acid sequences of polypeptides, the coupler is used that can recognize and bind to C-terminal amino acids of polypeptides. The transfer of the identifying information can be achieved by any suitable means such as by extension or ligation. In some embodiments, a spacer is added to the end of the recording tag, and the spacer comprises a sequence that is capable of hybridizing with a sequence on the coding tag to facilitate transfer of the identifying information.

Coding tag information associated with a specific binding agent may be transferred to a recording tag using a variety of methods. In certain embodiments, information of a coding tag is transferred to a recording tag via primer extension (Chan et al. (2015) Curr Opin Chem Biol 26: 55-61). A spacer sequence on the 3′-terminus of a recording tag or an extended recording tag anneals with complementary spacer sequence on the 3′ terminus of a coding tag and a polymerase (e.g., strand-displacing polymerase) extends the recording tag sequence, using the annealed coding tag as a template. In some embodiments, oligonucleotides complementary to coding tag encoder sequence and 5′ spacer can be pre-annealed to the coding tags to prevent hybridization of the coding tag to internal encoder and spacer sequences present in an extended recording tag. The 3′ terminal spacer, on the coding tag, remaining single stranded, preferably binds to the terminal 3′ spacer on the recording tag. In other embodiments, a nascent recording tag can be coated with a single stranded binding protein to prevent annealing of the coding tag to internal sites. Alternatively, the nascent recording tag can also be coated with RecA (or related homologues such as uvsX) to facilitate invasion of the 3′ terminus into a completely double stranded coding tag (Bell et al., 2012, Nature 491:274-278). This configuration prevents the double stranded coding tag from interacting with internal recording tag elements, yet is susceptible to strand invasion by the RecA coated 3′ tail of the extended recording tag (Bell, et al., 2015, Elife 4: e08646). The presence of a single-stranded binding protein can facilitate the strand displacement reaction.

The extended nucleic acid (e.g., recording tag) is any nucleic acid molecule or sequenceable polymer molecule (see, e.g., Niu et al., 2013, Nat. Chem. 5:282-292; Roy et al., 2015, Nat. Commun. 6:7237; Lutz, 2015, Polypeptides 48:4759-4767; each of which are incorporated by reference in its entirety) that comprises identifying information for a polypeptide, e.g., a polypeptide. In certain embodiments, after a binding agent binds to a polypeptide, information from a coding tag linked to a binding agent can be transferred to the nucleic acid associated with the polypeptide while the binding agent is bound to the isolated amino acid (e.g. amino acid in complex with the binding agent).

An extended recording tag associated with the polypeptide may comprise information from binding agent's coding tags representing each binding cycle performed. However, in some cases, an extended recording tag may also experience a “missed” binding cycle, e.g., if a binding agent fails to bind to the isolated amino acid, because the coding tag was missing, damaged, or defective, because the primer extension reaction failed. Even if a binding event occurs, transfer of information from the coding tag may be incomplete or less than 100% accurate, e.g., because a coding tag was damaged or defective, because errors were introduced in the primer extension reaction). Thus, an extended recording tag may represent 100%, or up to 95%, 90%, 85%, 80%, 75%, 70%, 65%, 60%, 65%, 55%, 50%, 45%, 40%, 35%, 30%, or any subrange thereof, of binding events that have occurred on its associated polypeptide. Moreover, the coding tag information present in the extended recording tag may have at least 30%, 35%, 40%, 45%, 50%, 55%, 60%, 65%, 70%, 75%, 80%, 85%, 90%, 95%, or 100% identity the corresponding coding tags.

In certain embodiments, an extended recording tag associated with the immobilized peptide may comprise information from multiple coding tags representing multiple, successive binding events. In these embodiments, a single, concatenated extended recording tag associated with the immobilized polypeptide can be representative of a single polypeptide. As referred to herein, transfer of coding tag information to the recording tag associated with the immobilized peptide also includes transfer to an extended recording tag as would occur in methods involving multiple, successive binding events.

In any of the preceding embodiments, the transfer of identifying information (e.g., from a coding tag to a recording tag) can be accomplished by ligation (e.g., an enzymatic or chemical ligation, a splint ligation, a sticky end ligation, a single-strand (ss) ligation such as a ssDNA ligation, or any combination thereof), a polymerase-mediated reaction (e.g., primer extension of single-stranded nucleic acid or double-stranded nucleic acid), or any combination thereof.

In some embodiments, a DNA polymerase that is used for primer extension possesses strand-displacement activity and has limited or is devoid of 3′-5 exonuclease activity. Several of many examples of such polymerases include Klenow exo− (Klenow fragment of DNA Pol 1), T4 DNA polymerase exo−, T7 DNA polymerase exo (Sequenase 2.0), Pfu exo−, Vent exo−, Deep Vent exo−, Bst DNA polymerase large fragment exo−, Bca Pol, 9° N Pol, and Phi29 Pol exo−. In a preferred embodiment, the DNA polymerase is active at room temperature and up to 45° C. In another embodiment, a “warm start” version of a thermophilic polymerase is employed such that the polymerase is activated and is used at about 40° C.-50° C. An exemplary warm start polymerase is Bst 2.0 Warm Start DNA Polymerase (New England Biolabs).

Additives useful in strand-displacement replication include any of a number of single-stranded DNA binding proteins (SSB proteins) of bacterial, viral, or eukaryotic origin, such as SSB protein of E. coli, phage T4 gene 32 product, phage T7 gene 2.5 protein, phage Pf3 SSB, replication protein A RPA32 and RPA14 subunits (Wold, Annu. Rev. Biochem. (1997) 66:61-92); other DNA binding proteins, such as adenovirus DNA-binding protein, herpes simplex protein ICP8, BMRF1 polymerase accessory subunit, herpes virus UL29 SSB-like protein; any of a number of replication complex proteins known to participate in DNA replication, such as phage T7 helicase/primase, phage T4 gene 41 helicase, E. coli Rep helicase, E. coli recBCD helicase, recA, E. coli and eukaryotic topoisomerases (Annu Rev Biochem. (2001) 70:369-413).

Mis-priming or self-priming events, such as when the terminal spacer sequence of the recording tag primes extension self-extension may be minimized by inclusion of single stranded binding proteins (T4 gene 32, E. coli SSB, etc.), DMSO (1-10%), formamide (1-10%), BSA (10-100 ug/ml), TMACl (1-5 mM), ammonium sulfate (10-50 mM), betaine (1-3 M), glycerol (5-40%), or ethylene glycol (5-40%), in the primer extension reaction.

Most type A polymerases are devoid of 3′ exonuclease activity (endogenous or engineered removal), such as Klenow exo-, T7 DNA polymerase exo− (Sequenase 2.0), and Taq polymerase catalyzes non-templated addition of a nucleotide, preferably an adenosine base (to lesser degree a G base, dependent on sequence context) to the 3′ blunt end of a duplex amplification product. For Taq polymerase, a 3′ pyrimidine (C>T) minimizes non-templated adenosine addition, whereas a 3′ purine nucleotide (G>A) favours non-templated adenosine addition. In some embodiments, using Taq polymerase for primer extension, placement of a thymidine base in the coding tag between the spacer sequence distal from the binding agent and the adjacent barcode sequence (e.g., encoder sequence or cycle specific sequence) accommodates the sporadic inclusion of a non-templated adenosine nucleotide on the 3′ terminus of the spacer sequence of the recording tag. In this manner, the extended recording tag associated with the immobilized peptide (with or without a non-templated adenosine base) can anneal to the coding tag and undergo primer extension.

Alternatively, addition of non-templated base can be reduced by employing a mutant polymerase (mesophilic or thermophilic) in which non-templated terminal transferase activity has been greatly reduced by one or more point mutations, especially in the 0-helix region (see U.S. Pat. No. 7,501,237) (Yang et al., Nucleic Acids Res. (2002) 30(19): 4314-4320). Pfu exo-, which is 3′ exonuclease deficient and has strand-displacing ability, also does not have non-templated terminal transferase activity.

In another embodiment, polymerase extension buffers are comprised of 40-120 mM buffering agent such as Tris-Acetate, Tris-HCl, HEPES, etc. at a pH of 6-9.

In certain embodiments, the annealing of the spacer sequence on the recording tag to the complementary spacer sequence on the coding tag is metastable under the primer extension reaction conditions (i.e., the annealing Tm is similar to the reaction temperature). This allows the spacer sequence of the coding tag to displace any blocking oligonucleotide annealed to the spacer sequence of the recording tag (or extensions thereof).

Coding tag information associated with a specific binding agent may be transferred to a nucleic acid on the recording tag associated with the immobilized peptide via ligation. Ligation may be a blunt end ligation or sticky end ligation. Ligation may be an enzymatic ligation reaction. Examples of ligases include, but are not limited to CV DNA ligase, T4 DNA ligase, T7 DNA ligase, T3 DNA ligase, Taq DNA ligase, E. coli DNA ligase, 9° N DNA ligase, Electroligase® (See e.g., U.S. Patent Publication No. US20140378315). Alternatively, a ligation may be a chemical ligation reaction, such as chemical ligation using standard chemical ligation or “click chemistry” (Gunderson et al., Genome Res (1998) 8(11): 1142-1153; Peng et al., European J Org Chem (2010) (22): 4194-4197; El-Sagheer et al., Proc Natl Acad Sci USA (2011) 108(28): 11338-11343; El-Sagheer et al., Org Biomol Chem (2011) 9(1): 232-235; Sharma et al., Anal Chem (2012) 84(14): 6104-6109; Roloff et al., Bioorg Med Chem (2013) 21(12): 3458-3464; Litovchick et al., Artif DNA PNA XNA (2014) 5(1): e27896; Roloff et al., Methods Mol Biol (2014) 1050:131-141).

In another embodiment, transfer of PNAs can be accomplished with chemical ligation using published techniques. The structure of PNA is such that it has a 5′ N-terminal amine group and an unreactive 3′ C-terminal amide. Chemical ligation of PNA requires that the termini be modified to be chemically active. This is typically done by derivatizing the 5′ N-terminus with a cysteinyl moiety and the 3′ C-terminus with a thioester moiety. Such modified PNAs easily couple using standard native chemical ligation conditions (Roloff et al., (2013) Bioorgan. Med. Chem. 21:3458-3464).

In some embodiments, coding tag information can be transferred using topoisomerase. Topoisomerase can be used be used to ligate a topo-charged 3′ phosphate on the recording tag (or extensions thereof or any nucleic acids attached) to the 5′ end of the coding tag, or complement thereof (Shuman et al., 1994, J. Biol. Chem. 269:32678-32684).

In certain embodiments, the binding event information is transferred from a coding tag to the recording tag associated with the immobilized peptide in a cyclic fashion. Cross-reactive binding events can be informatically filtered out after sequencing by requiring that at least two different coding tags, identifying two or more independent binding events, map to the same class of binding agents (cognate to a particular amino acid). The coding tag may contain an optional UMI sequence in addition to one or more spacer sequences. Universal priming sequences may also be included in extended nucleic acids on the recording tag associated with the immobilized peptide for amplification and NGS sequencing.

In some embodiments, the final extended recording tag containing information from one or more binding agents is optionally flanked by universal priming sites to facilitate downstream amplification and/or DNA sequencing. The forward universal priming site (e.g., Illumina's P5-S1 sequence) can be part of the original design of the recording tag and the reverse universal priming site (e.g., Illumina's P7-S2′ sequence) can be added as a final step in the extension of the nucleic acid. In some embodiments, the addition of forward and reverse priming sites can be done independently of a binding agent.

Recording Tag

In some embodiments, the target polypeptide may be labeled with a nucleic acid molecule or a oligonucleotide (e.g., DNA recording tag). In some aspects, a plurality of target polypeptides in the sample is provided with recording tags. The recording tags may be associated or attached, directly or indirectly to the target polypeptides using any suitable means. In some embodiments, a polypeptide may be associated with one or more recording tags. In some aspects, the recording tag may be any suitable sequenceable moiety to which identifying information can be transferred (e.g., information from one or more coding tags).

In some embodiments, at least one recording tag is associated or co-localized directly or indirectly with the target polypeptide. In a particular embodiment, a single recording tag is attached to a polypeptide, such as via the attachment to a N- or C-terminal amino acid. In another embodiment, multiple recording tags are attached to the polypeptide, such as to the lysine residues or peptide backbone. In some embodiments, a polypeptide labeled with multiple recording tags is fragmented or digested into smaller peptides, with each peptide labeled on average with one recording tag.

A recording tag may comprise DNA, RNA, or polynucleotide analogs including PNA, gPNA, GNA, HNA, BNA, XNA, TNA, or a combination thereof. A recording tag may be single stranded, or partially or completely double stranded. A recording tag may have a blunt end or overhanging end. In certain embodiments, all or a substantial amount of the polypeptides (e.g., at least 50%, 55%, 60%, 65%, 70%, 75%, 80%, 85%, 90%, 95%, 96%, 97%, 98%, 99%, or 100%) within a sample are labeled with a recording tag. In other embodiments, a subset of polypeptides within a sample are labeled with recording tags. In a particular embodiment, a subset of polypeptides from a sample undergo targeted (analyte specific) labeling with recording tags. For example, targeted recording tag labeling of proteins may be achieved using target protein-specific binding agents (e.g., antibodies, aptamers, etc.). In some embodiments, the recording tags are attached to the polypeptides prior to providing the sample on a support. In some embodiments, the recording tags are attached to the polypeptides after providing the sample on the support.

In some embodiments, the recording tag may comprise other nucleic acid components. In some embodiments, the recording tag may comprise a unique molecular identifier, a compartment tag, a partition barcode, sample barcode, a fraction barcode, a spacer sequence, a universal priming site, or any combination thereof. In some embodiments, the recording tag may comprise a blocking group, such as at the 3′-terminus of the recording tag. In some cases, the 3′-terminus of the recording tag is blocked to prevent extension of the recording tag by a polymerase.

In some embodiments, the recording tag can include a sample identifying barcode. A sample barcode is useful in the multiplexed analysis of a set of samples in a single reaction vessel or immobilized to a single solid substrate or collection of solid substrates (e.g., a planar slide, population of beads contained in a single tube or vessel, etc.). For example, polypeptides from many different samples can be labeled with recording tags with sample-specific barcodes, and then all the samples pooled together prior to immobilization to a support, cyclic binding of the binding agent, and recording tag analysis. Alternatively, the samples can be kept separate until after creation of a DNA-encoded library, and sample barcodes attached during PCR amplification of the DNA-encoded library, and then mixed together prior to sequencing. This approach could be useful when assaying analytes (e.g., proteins) of different abundance classes.

In certain embodiments, a recording tag comprises an optional, unique molecular identifier (UMI), which provides a unique identifier tag for each polypeptides (e.g., polypeptide) to which the UMI is associated with. In some embodiments, within a library of polypeptides, each polypeptide is associated with a single recording tag, with each recording tag comprising a unique UMI. In other embodiments, multiple copies of a recording tag are associated with a single polypeptide, with each copy of the recording tag comprising the same UMI. In some embodiments, a UMI has a different base sequence than the spacer or encoder sequences within the binding agents' coding tags to facilitate distinguishing these components during sequence analysis. In some embodiments, the UMI may provide function as a location identifier and also provide information in the polypeptide analysis assay. For example, the UMI may be used to identify molecules that are identical by descent, and therefore originated from the same initial molecule. In some aspects, this information can be used to correct for variations in amplification, and to detect and correct sequencing errors.

In some embodiments, the recording tags associated with a library of polypeptides share a common spacer sequence. In other embodiments, the recording tags associated with a library of polypeptides have binding cycle specific spacer sequences that are complementary to the binding cycle specific spacer sequences of their cognate binding agents. In some aspects, the spacer sequence in the recording tag is designed to have minimal complementarity to other regions in the recording tag; likewise, the spacer sequence in the coding tag should have minimal complementarity to other regions in the coding tag. In some cases, the spacer sequence of the recording tags and coding tags should have minimal sequence complementarity to components such unique molecular identifiers, barcodes (e.g., compartment, partition, sample, spatial location), universal primer sequences, encoder sequences, cycle specific sequences, etc. present in the recording tags or coding tags.

In certain embodiments, a recording tag comprises a universal priming site, e.g., a forward or 5′ universal priming site. A universal priming site is a nucleic acid sequence that may be used for priming a library amplification reaction and/or for sequencing. A universal priming site may include, but is not limited to, a priming site for PCR amplification, flow cell adaptor sequences that anneal to complementary oligonucleotides on flow cell surfaces (e.g., Illumina next generation sequencing), a sequencing priming site, or a combination thereof. A universal priming site can be about 10 bases to about 60 bases. In some embodiments, a universal priming site comprises an Illumina P5 primer (5′-AATGATACGGCGACCACCGA-3′—SEQ ID NO: 8) or an Illumina P7 primer (5′-CAAGCAGAAGACGGCATACGAGAT-3′—SEQ ID NO: 9).

In some embodiments, information of one or more tags are transferred to the recording tag (e.g., via primer extension or ligation) to extend the recording tag. In some embodiments, one or more of the tags (e.g., compartment tag, a partition barcode, sample barcode, a fraction barcode, etc.) further comprise a functional moiety capable of reacting with an internal amino acid, the peptide backbone, or N-terminal amino acid on the plurality of protein complexes, proteins, or polypeptides. In some embodiments, the functional moiety is a click chemistry moiety, an aldehyde, an azide/alkyne, or a maleimide/thiol, or an epoxide/nucleophile, an inverse electron demand Diels-Alder (iEDDA) group, or a moiety for a Staudinger reaction. In some specific embodiments, a plurality of compartment tags is formed by printing, spotting, ink-jetting the compartment tags into the compartment, or a combination thereof. In some embodiments, the tag is attached to a polypeptide to link the tag to the polypeptide via a polypeptide-polypeptide linkage. In some embodiments, the tag-attached polypeptide comprises a protein ligase recognition sequence.

In certain embodiments, a polypeptide can be immobilized to a support by an affinity capture reagent (and optionally covalently crosslinked), wherein the recording tag is associated with the affinity capture reagent directly, or alternatively, the polypeptide can be directly immobilized to the support with a recording tag. In one embodiment, the polypeptide is attached to a bait nucleic acid which hybridizes to a capture nucleic acid and is ligated to a capture nucleic acid which comprises a reactive coupling moiety for attaching to the support. In some embodiments, the bait or capture nucleic acid may serve as a recording tag to which information regarding the polypeptide can be transferred. In some embodiments, the polypeptide is attached to a bait nucleic acid to form a nucleic acid-polypeptide conjugate. In some embodiments, the immobilization methods comprise bringing the nucleic acid-polypeptide conjugate into proximity with a support by hybridizing the bait nucleic acid to a capture nucleic acid attached to the support, and covalently coupling the nucleic acid-polypeptide conjugate to the solid support. In some cases, the nucleic acid-polypeptide conjugate is coupled indirectly to the solid support, such as via a linker. In some embodiments, a plurality of the nucleic acid-polypeptide conjugates is coupled on the solid support and any adjacently coupled nucleic acid-polypeptide conjugates are spaced apart from each other at an average distance of about 50 nm or greater.

In some embodiments, the density or number of polypeptides provided with a recording tag is controlled or titrated. In some embodiments, the desired spacing, density, and/or amount of recording tags in the sample may be titrated by providing a diluted or controlled number of recording tags. In some embodiments, the desired spacing, density, and/or amount of recording tags may be achieved by spiking a competitor or “dummy” competitor molecule when providing, associating, and/or attaching the recording tags. In some cases, the “dummy” competitor molecule reacts in the same way as a recording tag being associated or attached to a polypeptide in the sample but the competitor molecule does not function as a recording tag. In some specific examples, if a desired density is 1 functional recording tag per 1,000 available sites for attachment in the sample, then spiking in 1 functional recording tag for every 1,000 “dummy” competitor molecules is used to achieve the desired spacing. In some embodiments, the ratio of functional recording tags is adjusted based on the reaction rate of the functional recording tags compared to the reaction rate of the competitor molecules.

In some embodiments, the labeling of the polypeptide with a recording tag is performed using standard amine coupling chemistries. For example, the e-amino group (e.g., of lysine residues) and the N-terminal amino group may be susceptible to labeling with amine-reactive coupling agents, depending on the pH of the reaction (Mendoza et al., Mass Spectrom Rev (2009) 28(5): 785-815). In a particular embodiment, the recording tag comprises a reactive moiety (e.g., for conjugation to a solid surface, a multifunctional linker, or a polypeptide), a linker, a universal priming sequence, a barcode (e.g., compartment tag, partition barcode, sample barcode, fraction barcode, or any combination thereof), an optional UMI, and a spacer (Sp) sequence for facilitating information transfer to/from a coding tag. In another embodiment, the protein can be first labeled with a universal DNA tag, and the barcode-Sp sequence (representing a sample, a compartment, a physical location on a slide, etc.) are attached to the protein later through and enzymatic or chemical coupling step. A universal DNA tag comprises a short sequence of nucleotides that are used to label a protein or polypeptide polypeptide and can be used as point of attachment for a barcode (e.g., compartment tag, recording tag, etc.). For example, a recording tag may comprise at its terminus a sequence complementary to the universal DNA tag. In certain embodiments, a universal DNA tag is a universal priming sequence. Upon hybridization of the universal DNA tags on the labeled protein to complementary sequence in recording tags (e.g., bound to beads), the annealed universal DNA tag may be extended via primer extension, transferring the recording tag information to the DNA tagged protein. In a particular embodiment, the protein is labeled with a universal DNA tag prior to proteinase digestion into peptides. The universal DNA tags on the labeled peptides from the digest can then be converted into an informative and effective recording tag.

The recording tags may comprise a reactive moiety for a cognate reactive moiety present on the target polypeptide, e.g., the target protein, (e.g., click chemistry labeling, photoaffinity labeling). For example, recording tags may comprise an azide moiety for interacting with alkyne-derivatized proteins, or recording tags may comprise a benzophenone for interacting with native proteins, etc. Upon binding of the target protein by the target protein specific binding agent, the recording tag and target protein are coupled via their corresponding reactive moieties. After the target protein is labeled with the recording tag, the target-protein specific binding agent may be removed by digestion of the DNA capture probe linked to the target-protein specific binding agent. For example, the DNA capture probe may be designed to contain uracil bases, which are then targeted for digestion with a uracil-specific excision reagent (e.g., USER™), and the target-protein specific binding agent may be dissociated from the target protein. In some embodiments, other types of linkages besides hybridization can be used to link the recording tag to a polypeptide. A suitable linker can be attached to various positions of the recording tag, such as the 3′ end, at an internal position, or within the linker attached to the 5′ end of the recording tag.

In some aspects, the spacer sequence in the recording is designed to have minimal complementarity to other regions in the recording tag; likewise, the spacer sequence in the coding tag should have minimal complementarity to other regions in the coding tag. In other words, the spacer sequence of the recording tags and coding tags should have minimal sequence complementarity to components such unique molecular identifiers, barcodes (e.g., compartment, partition, sample, spatial location), universal primer sequences, encoder sequences, cycle specific sequences, etc. present in the recording tags or coding tags.

In some embodiments, a recording tag comprises from 5′ to 3′ direction: a universal forward (or 5′) priming sequence, information transferred from the coding tag, and a spacer sequence. In some embodiments, an extended recording tag comprises from 5′ to 3′ direction: a universal forward (or 5′) priming sequence, information transferred from the coding tag, optionally other barcodes (e.g., sample barcode, partition barcode, compartment barcode, or any combination thereof), a spacer sequence, a universal reverse (or 3′) priming sequence. In some other embodiments, a recording tag comprises from 5′ to 3′ direction: a universal forward (or 5′) priming sequence, information transferred from the coding tag, optionally other barcodes (e.g., sample barcode, partition barcode, compartment barcode, or any combination thereof), an optional UMI, and a spacer sequence.

Coding Tag

The coding tag associated with the binding agent is or comprises a polynucleotide with any suitable length, e.g., a nucleic acid molecule of about 2 bases to about 100 bases, including any integer including 2 and 100 and in between, that comprises identifying information for its associated binding agent. A coding tag may comprise an encoder sequence or a sequence with identifying information, which is optionally flanked by one spacer on one side or optionally flanked by a spacer on each side. A coding tag may also be comprised of an optional UMI and/or an optional binding cycle-specific barcode. A coding tag may refer to the coding tag that is directly attached to a binding agent, to a complementary sequence hybridized to the coding tag directly attached to a binding agent (e.g., for double stranded coding tags), or to coding tag information present in an extended nucleic acid on the recording tag. In certain embodiments, a coding tag may further comprise a binding cycle specific spacer or barcode, a unique molecular identifier, a universal priming site, or any combination thereof.

A coding tag may be a single stranded molecule, a double stranded molecule, or a partially double stranded. A coding tag may comprise blunt ends, overhanging ends, or one of each. In some embodiments, a coding tag is partially double stranded, which prevents annealing of the coding tag to internal encoder and spacer sequences in a growing extended recording tag. In some embodiments, the coding tag may comprise a hairpin. In certain embodiments, the hairpin comprises mutually complementary nucleic acid regions are connected through a nucleic acid strand. In some embodiments, the nucleic acid hairpin can also further comprise 3′ and/or 5′ single-stranded region(s) extending from the double-stranded stem segment. In some embodiments, the hairpin comprises a single strand of nucleic acid.

In some embodiments, a binding agent described comprises a coding tag containing identifying information regarding the binding agent. In some embodiments, the identifying information from the coding tag comprises information regarding the identity of the target bound by the binding agent. In some embodiments, the identifying information from the coding tag comprises information regarding the identity of the terminal amino acid of the polypeptide bound by the binding agent.

In some embodiments, each unique binding agent within a library of binding agents has a unique encoder sequence. For example, 20 unique encoder sequences may be used for a library of 20 binding agents that bind to the 20 standard amino acids. Additional coding tag sequences may be used to identify modified amino acids (e.g., post-translationally modified amino acids). In another example, 30 unique encoder sequences may be used for a library of 30 binding agents that bind to the 20 standard amino acids and 10 post-translational modified amino acids (e.g., phosphorylated amino acids, acetylated amino acids, methylated amino acids). In other embodiments, two or more different binding agents may share the same encoder sequence. For example, two binding agents that each bind to a different standard amino acid may share the same encoder sequence.

In some embodiments, the coding tags within a collection of binding agents share a common spacer sequence used in an assay (e.g. the entire library of binding agents used in a multiple binding cycle method possess a common spacer in their coding tags). In another embodiment, the coding tags are comprised of a binding cycle tags, identifying a particular binding cycle. In other embodiments, the coding tags within a library of binding agents have a binding cycle specific spacer sequence. In some embodiments, a coding tag comprises one binding cycle specific spacer sequence. For example, a coding tag for binding agents used in the first binding cycle comprise a “cycle 1” specific spacer sequence, a coding tag for binding agents used in the second binding cycle comprise a “cycle 2” specific spacer sequence, and so on up to “n” binding cycles. In further embodiments, coding tags for binding agents used in the first binding cycle comprise a “cycle 1” specific spacer sequence and a “cycle 2” specific spacer sequence, coding tags for binding agents used in the second binding cycle comprise a “cycle 2” specific spacer sequence and a “cycle 3” specific spacer sequence, and so on up to “n” binding cycles. In some embodiments, a spacer sequence comprises a sufficient number of bases to anneal to a complementary spacer sequence in a recording tag or extended recording tag to initiate a primer extension reaction or sticky end ligation reaction.

A cycle specific spacer sequence in the coding tag can be used to concatenate information of coding tags onto a single recording tag when a population of recording tags is associated with a polypeptide. The first binding cycle transfers information from the coding tag to a randomly-chosen recording tag, and subsequent binding cycles can prime only the extended recording tag using cycle dependent spacer sequences. More specifically, coding tags for binding agents used in the first binding cycle comprise a “cycle 1” specific spacer sequence and a “cycle 2” specific spacer sequence, coding tags for binding agents used in the second binding cycle comprise a “cycle 2” specific spacer sequence and a “cycle 3” specific spacer sequence, and so on up to “n” binding cycles. Coding tags of binding agents from the first binding cycle are capable of annealing to recording tags via complementary cycle 1 specific spacer sequences. Upon transfer of the coding tag information to the recording tag, the cycle 2 specific spacer sequence is positioned at the 3′ terminus of the extended recording tag at the end of binding cycle 1. Coding tags of binding agents from the second binding cycle are capable of annealing to the extended recording tags via complementary cycle 2 specific spacer sequences. Upon transfer of the coding tag information to the extended recording tag, the cycle 3 specific spacer sequence is positioned at the 3′ terminus of the extended recording tag at the end of binding cycle 2, and so on through “n” binding cycles. This embodiment provides that transfer of binding information in a particular binding cycle among multiple binding cycles will only occur on (extended) recording tags that have experienced the previous binding cycles. However, sometimes a binding agent may fail to bind to a cognate polypeptide. Oligonucleotides comprising binding cycle specific spacers after each binding cycle as a “chase” step can be used to keep the binding cycles synchronized even if the event of a binding cycle failure. For example, if a cognate binding agent fails to bind to a polypeptide during binding cycle 1, adding a chase step following binding cycle 1 using oligonucleotides comprising both a cycle 1 specific spacer, a cycle 2 specific spacer, and a “null” encoder sequence. The “null” encoder sequence can be the absence of an encoder sequence or, preferably, a specific barcode that positively identifies a “null” binding cycle. The “null” oligonucleotide is capable of annealing to the recording tag via the cycle 1 specific spacer, and the cycle 2 specific spacer is transferred to the recording tag. Thus, binding agents from binding cycle 2 are capable of annealing to the extended recording tag via the cycle 2 specific spacer despite the failed binding cycle 1 event. The “null” oligonucleotide marks binding cycle 1 as a failed binding event within the extended recording tag.

An extended recording tag can be built up from a series of binding events using coding tags comprising analyte-specific spacers and encoder sequences. In one embodiment, a first binding event employs a binding agent with a coding tag comprised of a generic 3′ spacer primer sequence and an analyte-specific spacer sequence at the 5′ terminus for use in the next binding cycle; subsequent binding cycles then use binding agents with encoded analyte-specific 3′ spacer sequences. This design results in amplifiable library elements being created only from a correct series of cognate binding events. Off-target and cross-reactive binding interactions will lead to a non-amplifiable extended recording tag. In one example, a pair of cognate binding agents to a particular polypeptide analyte is used in two binding cycles. The first cognate binding agent contains a coding tag comprised of a generic spacer 3′ sequence for priming extension on the generic spacer sequence of the recording tag, and an encoded analyte-specific spacer at the 5′ end, which will be used in the next binding cycle. For matched cognate binding agent pairs, the 3′ analyte-specific spacer of the second binding agent is matched to the 5′ analyte-specific spacer of the first binding agent. In this way, only correct binding of the cognate pair of binding agents will result in an amplifiable extended recording tag. Cross-reactive binding agents will not be able to prime extension on the recording tag, and no amplifiable extended recording tag product generated. This approach greatly enhances the specificity of the methods disclosed herein. The same principle can be applied to triplet binding agent sets, in which 3 cycles of binding are employed. In a first binding cycle, a generic 3′ Sp sequence on the recording tag interacts with a generic spacer on a binding agent coding tag. Primer extension transfers coding tag information, including an analyte specific 5′ spacer, to the recording tag. Subsequent binding cycles employ analyte specific spacers on the binding agents' coding tags.

A coding tag may include a terminator nucleotide incorporated at the 3′ end of the 3′ spacer sequence. After a binding agent binds to a polypeptide and their corresponding coding tag and recording tags anneal via complementary spacer sequences, it is possible for primer extension to transfer information from the coding tag to the recording tag, or to transfer information from the recording tag to the coding tag. Addition of a terminator nucleotide on the 3′ end of the coding tag prevents transfer of recording tag information to the coding tag. It is understood that for embodiments described herein involving generation of extended coding tags, it may be preferable to include a terminator nucleotide at the 3′ end of the recording tag to prevent transfer of coding tag information to the recording tag.

A coding tag can be joined to a binding agent directly or indirectly, by any means known in the art, including covalent and non-covalent interactions. In some embodiments, a coding tag may be joined to binding agent enzymatically or chemically. In some embodiments, a binding agent is joined to a coding tag via SpyCatcher-SpyTag interaction. The SpyTag peptide forms an irreversible covalent bond to the SpyCatcher protein via a spontaneous isopeptide linkage, thereby offering a genetically encoded way to create peptide interactions that resist force and harsh conditions (Zakeri et al., 2012, Proc. Natl. Acad. Sci. 109:E690-697; Li et al., 2014, J. Mol. Biol. 426:309-317). A binding agent may be expressed as a fusion protein comprising the SpyCatcher protein. In some embodiments, the SpyCatcher protein is appended on the N-terminus or C-terminus of the binding agent. The SpyTag peptide can be coupled to the coding tag using standard conjugation chemistries (Hermanson, Bioconjugate Techniques, (2013) Academic Press).

In other embodiments, a binding agent is joined to a coding tag via SnoopTag-SnoopCatcher peptide-protein interaction. The SnoopTag peptide forms an isopeptide bond with the SnoopCatcher protein (Veggiani et al., Proc. Natl. Acad. Sci. USA, 2016, 113:1202-1207). A binding agent may be expressed as a fusion protein comprising the SnoopCatcher protein. In some embodiments, the SnoopCatcher protein is appended on the N-terminus or C-terminus of the binding agent. The SnoopTag peptide can be coupled to the coding tag using standard conjugation chemistries.

In certain embodiments, a coding tag may further comprise a unique molecular identifier for the binding agent to which the coding tag is linked. A UMI for the binding agent may be useful in embodiments utilizing extended coding tags or di-tag molecules for sequencing readouts, which in combination with the encoder sequence provides information regarding the identity of the binding agent and number of unique binding events for a polypeptide.

Amino Acid Cleavage

In embodiments relating to methods of analyzing target polypeptides using a degradation-based approach, following contacting, tethering and cleaving off the coupler with the NTAA, followed by binding of a first binding agent to an isolated NTAA of the polypeptide of n amino acids, and transferring of the first binding agent's coding tag information to a nucleic acid associated with the polypeptide, thereby generating a first order extended recording tag, the new NTAA of the polypeptide is exposed and the isolated NTAA in complex with the coupler is released. In the next cycle, the coupler is contacted with the newly exposed NTAA, followed by tethering and cleaving off the coupler with the NTAA, followed by binding of a second binding agent comprising a second coding tag with identifying information regarding the second binding agent to the isolated coupler-NTAA complex. In this cycle, information from the second coding tag is transferred to the first order extended recording tag, thereby generating a second order extended recording tag. Additional cycles of coupler binding, tethering, cleaving off, binding and information transfer can occur as described above up to n amino acids to generate an n^{th} order extended recording tag or n separate extended recording tags, which collectively represent the polypeptide. In some embodiments, steps including the NTAA in the described exemplary approach can be performed instead with the C-terminal amino acid (CTAA).

Cleavage of a terminal amino acid in complex with the coupler can be accomplished by any number of known techniques, including chemical cleavage and enzymatic cleavage. In some embodiments, an engineered enzyme that catalyzes or reagent that promotes the removal of the PITC-derivatized or other labeled N-terminal amino acid is used. In some embodiments, the terminal amino acid in complex with the coupler is removed or eliminated using any of the methods as described in published US patent publication US 20200348307 A1. In some embodiments, cleavage of a terminal amino uses a carboxypeptidase, an aminopeptidase, a dipeptidyl peptidase, a dipeptidyl aminopeptidase or a variant, mutant, or modified protein thereof, a hydrolase or a variant, mutant, or modified protein thereof, a mild Edman degradation reagent; an Edmanase enzyme; anhydrous TFA, a base; or any combination thereof. In some embodiments, the mild Edman degradation uses a dichloro or monochloro acid; the mild Edman degradation uses TFA, TCA, or DCA; or the mild Edman degradation uses triethylamine, triethanolamine, or triethylammonium acetate (Et₃NHOAc).

Preferred enzymatic cleavage of a NTAA in complex with the coupler may be accomplished by a modified aminopeptidase. Aminopeptidases naturally occur as monomeric and multimeric enzymes, and may be metal or ATP-dependent. Natural aminopeptidases have very limited specificity, and generically cleave N-terminal amino acids in a processive manner, cleaving one amino acid off after another. For the methods described here, aminopeptidases (e.g., M29 metalloenzymatic aminopeptidase) may be engineered to possess specific binding or catalytic activity to the NTAA only when modified with the coupler. Exemplar members of the M29 aminopeptidase family include, Mesoamp, Aminopeptidase T, Leucine Aminopeptidase, BsAmpII, PseA PepB, Aquifex aeolicus thermostable aminopeptidase, Aminopeptidase PepS, leucine aminopeptidase LAP, and homologues thereof (Sierra, E. M., et al. Halotolerant aminopeptidase M29 from Mesorhizobium SEMIA 3007 with biotechnological potential and its impact on biofilm synthesis. Sci Rep (2017) 7, 10684). For example, an aminopeptidase may be engineered such than it only cleaves an N-terminal amino acid if it is modified by a group such as isothiocyanate, 2-Aminobenzoyl (Abz), 2,4-dinitrophenyl (DNP), guanidinyl, diheterocyclic methanimine. In this way, the aminopeptidase cleaves only a single terminal amino acid in complex with the coupler at a time, and allows control of the degradation cycle. In some embodiments, the modified aminopeptidase is non-selective as to amino acid residue identity while being selective for the coupler. In other embodiments, the modified aminopeptidase is selective for both amino acid residue identity and the coupler. Engineered aminopeptidase variants that bind to and cleave individual labelled NTAA residues have been described (see, US 2021/0214701 A1; PCT Publication No. WO2010/065322).

In another example, Havranak et al. (U.S. Patent Publication No. US 2014/0273004) describes engineering aminoacyl tRNA synthetases (aaRSs) as specific NTAA binding agents. The amino acid binding pocket of the aaRSs has an intrinsic ability to bind cognate amino acids (Kaiser F, et al., The structural basis of the genetic code: amino acid recognition by aminoacyl-tRNA synthetases. Sci Rep. 2020 Jul. 28; 10(1):12647), but generally exhibits poor binding affinity and specificity in the context of peptides (Borgo, Benjamin, “Strategies for Computational Protein Design with Application to the Development of a Biomolecular Tool-kit for Single Molecule Protein Sequencing” (2014). All Theses and Dissertations (ETDs). 1221). Moreover, these natural amino acid binding agents don't recognize N-terminal labels. Directed evolution of aaRS scaffolds can be used to generate higher affinity, higher specificity binding agents that recognized the N-terminal amino acids in the context of an N-terminal label.

In certain embodiments, the aminopeptidase may be engineered to be non-specific, such that it does not selectively recognize one particular amino acid over another, but rather just recognizes the labeled N-terminus. In yet another embodiment, cyclic cleavage is attained by using an engineered acylpeptide hydrolase (APH) to cleave an acetylated NTAA. In yet another embodiment, amidination (guanidinylation) of the NTAA is employed to enable mild cleavage of the labeled NTAA using NaOH (Hamada, (2016) Bioorg Med Chem Lett 26(7): 1690-1695).

In some embodiments, the method further comprises contacting the polypeptide with a proline aminopeptidase under conditions suitable to cleave an N-terminal proline in complex with the coupler. In some embodiments, a proline aminopeptidase (PAP) is an enzyme that is capable of specifically cleaving an N-terminal proline from a polypeptide. PAP enzymes that cleave N-terminal prolines are also referred to as proline iminopeptidases (PIPs). Known monomeric PAPs include family members from B. coagulans, L. delbrueckii, N. gonorrhoeae, F. meningosepticum, S. marcescens, T. acidophilum, L. plantarum (MEROPS 533.001) Nakajima et al., J Bacteriol. (2006) 188(4):1599-606; Kitazono et al., Bacteriol (1992) 174(24):7919-7925). Known multimeric PAPs including D. hansenii (Bolumar et al., (2003) 86(1-2):141-151) and similar homologues from other species (Basten et al., Mol Genet Genomics (2005) 272(6):673-679). Either native or engineered variants/mutants of PAPs may be employed.

For embodiments relating to polypeptides immobilized on a solid support via the N-terminus, a coupler with a CTAA-binding moiety can be utilized, followed by the CTAA cleavage. For example, U.S. Pat. No. 6,046,053 discloses a method of reacting the polypeptide with an alkyl acid anhydride to convert the carboxy-terminal into oxazolone, liberating the C-terminal amino acid by reaction with acid and alcohol or with ester. Enzymatic cleavage of a CTAA may also be accomplished by a carboxypeptidase. Several carboxypeptidases exhibit amino acid preferences, e.g., carboxypeptidase B preferentially cleaves at basic amino acids, such as arginine and lysine. As described above, carboxypeptidases may also be modified in the same fashion as aminopeptidases to engineer carboxypeptidases that specifically bind to CTAAs having a C-terminal label (coupler). In this way, the carboxypeptidase cleaves only a single amino acid at a time from the C-terminus, and allows control of the degradation cycle. In some embodiments, the modified carboxypeptidase is non-selective as to amino acid residue identity while being selective for the C-terminal label (coupler). In other embodiments, the modified carboxypeptidase is selective for both amino acid residue identity and the coupler.

In some embodiments, the coupler comprises a C-terminal modification group (CTM) that is configured to react with the CTAA of the polypeptide and comprises one of the following moieties: to, isothiocyanate, diphenylphosphoryl isothiocyanate, tetrabutylammonium isothiocyanate, sodium thiocyanate, ammonium thiocyanate, acetyl chloride, and cyanogen bromide. In some embodiments, the CTAA of the polypeptide is contacted with the CTM group of the coupler under conditions that allow the CTAA to be conjugated to the carboxyl reactive moiety of the coupler to form a coupler-polypeptide complex.

Analysis

The methods disclosed herein can be used for high throughput analysis, including detection, quantitation and/or sequencing, of a plurality of polypeptide analytes simultaneously (multiplexing). Multiplexing as used herein refers to analysis of a plurality of polypeptide analytes in the same assay. The plurality of polypeptide analytes can be derived from the same sample or different samples. The plurality of polypeptide analytes can be derived from the same subject or different subjects. The plurality of polypeptide analytes that are analyzed can be different polypeptide analytes, or the same polypeptide analytes derived from different samples. A plurality of polypeptide analytes includes 5 or more polypeptides, 10 or more polypeptides, 100 or more polypeptides, 500 or more polypeptides, 1000 or more polypeptides, 5,000 or more polypeptides, 10,000 or more polypeptides, 50,000 or more polypeptides, or 100,000 or more polypeptides. Sample multiplexing can additionally be achieved by upfront barcoding of recording tag labeled polypeptide samples. Each barcode represents a different sample, and samples can be pooled prior to cyclic binding assays or sequence analysis. In this way, many barcode-labeled samples can be simultaneously processed in a single tube.

In preferred embodiments, analyzing the recording tag extended after information transfer comprises a nucleic acid sequencing method. In preferred embodiments, analyzing the recording tag extended after information transfer further comprises obtaining the identifying information regarding the binding agent that was bound to the coupler-amino acid complex to obtain information regarding the polypeptide analyte (in particular, information regarding the NTAA residue). Information about the polypeptide analyte is derived from the sequence of the extended recording tag, which accumulates binding history of the polypeptide analyte after each binding cycle via incorporating coding tag information that comprises identifying information regarding the binding agent. Sequencing the extended recording tag allows for retrieval of the identifying information regarding the binding agent(s) bound to the isolated coupler-amino acid complex, which provides information about the NTAA residue of the polypeptide analyte.

Identifying a polypeptide analyte by the disclosed methods can be accomplished by determining the sequence of a segment of the polypeptide or determining partial sequence information for the polypeptide. Partial sequencing of the polypeptide can be powerful and sufficient to discriminate polypeptide identity when mapped back to available genomic and proteomic databases. For example, it is possible to uniquely identify more than 90% of the human proteome by identifying six consecutive amino acid residues of the polypeptide, which can be accomplish via obtaining the identifying information regarding the binding agents that were bound to the corresponding coupler-amino acid complexes. In some embodiments of the disclosed methods, less selective binding agents may not provide exact identity of the NTAA, but instead provide a possible type of the NTAA, such as a type that includes two or more structurally similar amino acid residues (e.g., small hydrophobic residues, positively charged residues, etc.). Such type-specific identity information can still be sufficient to discriminate protein identity when mapped back to available genomic and proteomic databases.

In some embodiments, the extended recording tag generated from performing the provided methods comprises information transferred from one or more coding tags. In some embodiments, the extended recording tags further comprise identifying information from one or more coding tags. In some embodiments, the extended recording tags are amplified (or a portion thereof) prior to determining at least the sequence of the coding tag(s) in the extended recording tag. In some embodiments, the extended recording tags (or a portion thereof) are released prior to determining at least the sequence of the coding tag(s) in the extended recording tag.

The length of the final extended recording tag generated by the methods described herein is dependent upon multiple factors, including the length of the coding tag(s) (e.g., barcode and spacer), the length of the nucleic acids (e.g., optionally including any unique molecular identifier, spacer, universal priming site, barcode, or combinations thereof). After transfer of the final tag information to the extended nucleic acid (e.g., from any coding tags), the tag can be capped by addition of a universal reverse priming site via ligation, primer extension or other methods known in the art. In some embodiments, the universal forward priming site in the nucleic acid (e.g., on the recording tag) is compatible with the universal reverse priming site that is appended to the final extended nucleic acid. In some embodiments, a universal reverse priming site is an Illumina P7 primer (5′-CAAGCAGAAGACGGCATACGAGAT-3′—SEQ ID NO:9) or an Illumina P5 primer (5′-AATGATACGGCGACCACCGA-3′—SEQ ID NO:8). The sense or antisense P7 may be appended, depending on strand sense of the nucleic acid to which the identifying information from the coding tag is transferred to. An extended nucleic acid library can be cleaved or amplified directly from the support (e.g., beads) and used in traditional next generation sequencing assays and protocols.

In some embodiments, a primer extension reaction is performed on a library of single stranded extended nucleic acids (e.g., extended on the recording tag) to copy complementary strands thereof. In some embodiments, the peptide sequencing assay (e.g., ProteoCode™ assay), comprises several chemical and enzymatic steps in a cyclical progression.

Extended nucleic acids recording tags can be processed and analysed using a variety of nucleic acid sequencing methods. In some embodiments, extended recording tags containing the information from one or more coding tags and any other nucleic acid components are processed and analysed. In some embodiments, the collection of extended recording tags can be concatenated. In some embodiments, the extended recording tag can be amplified prior to determining the sequence.

In some embodiments, the recording tag or extended recording tag comprises information from one or more coding tags is analysed and/or sequenced. In some embodiments, the method includes analyzing the identifying information regarding the binding agent of the polypeptide analysis assay transferred to the recording tag.

Examples of sequencing methods include, but are not limited to, chain termination sequencing (Sanger sequencing); next generation sequencing methods, such as sequencing by synthesis, sequencing by ligation, sequencing by hybridization, polony sequencing, ion semiconductor sequencing, and pyrosequencing; and third generation sequencing methods, such as single molecule real time sequencing, nanopore-based sequencing, duplex interrupted sequencing, and direct imaging of DNA using advanced microscopy.

Suitable sequencing methods for use in the invention include, but are not limited to, sequencing by hybridization, sequencing by synthesis technology (e.g., HiSeq™ and Solexa™, Illumina), SMRT™ (Single Molecule Real Time) technology (Pacific Biosciences), true single molecule sequencing (e.g., HeliScope™, Helicos Biosciences), massively parallel next generation sequencing (e.g., SOLiD™, Applied Biosciences; Solexa and HiSeq™, Illumina), massively parallel semiconductor sequencing (e.g., Ion Torrent), pyrosequencing technology (e.g., GS FLX and GS Junior Systems, Roche/454), nanopore sequence (e.g., Oxford Nanopore Technologies).

A library of nucleic acids (e.g., recording tags comprising information from one or more coding tags) may be amplified in a variety of ways, for example, via PCR or emulsion PCR. Emulsion PCR is known to produce more uniform amplification (Hori, Fukano et al., Biochem Biophys Res Commun (2007) 352(2): 323-328). Alternatively, a library of nucleic acids (e.g., extended nucleic acids) may undergo linear amplification, e.g., via in vitro transcription of template DNA using T7 RNA polymerase. The library of nucleic acids (e.g., extended nucleic acids) can be amplified using primers compatible with the universal forward priming site and universal reverse priming site contained therein. A library of nucleic acids (e.g., the recording tag) can also be amplified using tailed primers to add sequence to either the 5′-end, 3′-end or both ends of the extended nucleic acids. Sequences that can be added to the termini of the extended nucleic acids include library specific index sequences to allow multiplexing of multiple libraries in a single sequencing run, adaptor sequences, read primer sequences, or any other sequences for making the library of extended nucleic acids compatible for a sequencing platform. An example of a library amplification in preparation for next generation sequencing is as follows: a 20 μl PCR reaction volume is set up using an extended nucleic acid library eluted from ˜1 mg of beads (˜10 ng), 200 μM dNTP, 1 μM of each forward and reverse amplification primers, 0.5 μl (1U) of Phusion Hot Start enzyme (New England Biolabs) and subjected to the following cycling conditions: 98° C. for 30 sec followed by 20 cycles of 98° C. for 10 sec, 60° C. for 30 sec, 72° C. for 30 sec, followed by 72° C. for 7 min, then hold at 4° C.

In certain embodiments, either before, during or following amplification, the library of nucleic acids (e.g., extended recording tags) can undergo target enrichment. In some embodiments, target enrichment can be used to selectively capture or amplify extended nucleic acids representing polypeptides of interest from a library of extended recording tags before sequencing. In some aspects, target enrichment for protein sequencing is challenging because of the high cost and difficulty in producing highly-specific binding agents. In some cases, antibodies are notoriously non-specific and difficult to scale production across thousands of proteins. In some embodiments, the methods of the present disclosure circumvent this problem by converting the protein code into a nucleic acid code which can then make use of a wide range of targeted DNA enrichment strategies available for DNA libraries. In some cases, polypeptides of interest can be enriched in a sample by enriching their corresponding extended recording tags. Methods of targeted enrichment are known in the art, and include hybrid capture assays, PCR-based assays such as TruSeq custom Amplicon (Illumina), padlock probes (also referred to as molecular inversion probes), and the like (see, Mamanova et al., (2010) Nature Methods 7: 111-118; Bodi et al., J. Biomol. Tech. (2013) 24:73-86; Ballester et al., (2016) Expert Review of Molecular Diagnostics 357-372; Mertes et al., (2011) Brief Funct. Genomics 10:374-386; Nilsson et al., (1994) Science 265:2085-8; each of which are incorporated herein by reference in their entirety).

In one embodiment, a library of nucleic acids (e.g., extended recording tags) is enriched via a hybrid capture-based assay. In a hybrid-capture based assay, the library of extended nucleic acids is hybridized to target-specific oligonucleotides that are labelled with an affinity tag (e.g., biotin). Extended nucleic acids hybridized to the target-specific oligonucleotides are “pulled down” via their affinity tags using an affinity ligand (e.g., streptavidin coated beads), and background (non-specific) extended nucleic acids are washed away. The enriched extended nucleic acids (e.g., extended recording tags) are then obtained for positive enrichment (e.g., eluted from the beads). In some embodiments, oligonucleotides complementary to the corresponding extended nucleic acid library representations of peptides of interest can be used in a hybrid capture assay. In some embodiments, sequential rounds or enrichment can also be carried out, with the same or different bait sets.

In another embodiment, primer extension and ligation-based mediated amplification enrichment (AmpliSeq, PCR, TruSeq TSCA, etc.) can be used to select and module fraction enriched of library elements representing a subset of polypeptides. Competing oligonucleotides can also be employed to tune the degree of primer extension, ligation, or amplification. In the simplest implementation, this can be accomplished by having a mix of target specific primers comprising a universal primer tail and competing primers lacking a 5′ universal primer tail. After an initial primer extension, only primers with the 5′ universal primer sequence can be amplified. The ratio of primer with and without the universal primer sequence controls the fraction of target amplified. In other embodiments, the inclusion of hybridizing but non-extending primers can be used to modulate the fraction of library elements undergoing primer extension, ligation, or amplification.

A competitor oligonucleotide bait, hybridizing to the target but lacking a biotin moiety, can also be used in the hybrid capture step to modulate the fraction of any particular locus enriched. The competitor oligonucleotide bait competes for hybridization to the target with the standard biotinylated bait effectively modulating the fraction of target pulled down during enrichment. The ten orders dynamic range of protein expression can be compressed by several orders using this competitive suppression approach, especially for the overly abundant species such as albumin. Thus, the fraction of library elements captured for a given locus relative to standard hybrid capture can be modulated from 100% down to 0% enrichment.

Additionally, library normalization techniques can be used to remove overly abundant species from the extended nucleic acid library. The ssDNA library elements can be separated from the abundant dsDNA library elements using methods known in the art, such as chromatography on hydroxyapatite columns (VanderNoot, et al., 2012, Biotechniques 53:373-380) or treatment of the library with a duplex-specific nuclease (DSN) from Kamchatka crab (Shagin et al., (2002) Genome Res. 12:1935-42) which destroys the dsDNA library elements.

In some embodiments, a library of nucleic acids (e.g., extended nucleic acids) is concatenated by ligation or end-complementary PCR to create a long DNA molecule comprising multiple different extended recorder tags, extended coding tags, or di-tags, respectively (Du et al., (2003) BioTechniques 35:66-72; Muecke et al., (2008) Structure 16:837-841; U.S. Pat. No. 5,834,252, each of which is incorporated by reference in its entirety). This embodiment is preferable for nanopore sequencing in which long strands of DNA are analyzed by the nanopore sequencing device.

In some embodiments, direct single molecule analysis is performed on the nucleic acids (e.g., extended nucleic acids) (see, e.g., Harris et al., (2008) Science 320:106-109). The nucleic acids (e.g., extended recording tags) can be analysed directly on the support, such as a flow cell or beads that are compatible for loading onto a flow cell surface (optionally microcell patterned), wherein the flow cell or beads can integrate with a single molecule sequencer or a single molecule decoding instrument. For single molecule decoding, hybridization of several rounds of pooled fluorescently-labelled of decoding oligonucleotides (Gunderson et al., (2004) Genome Res. 14:970-7) can be used to ascertain both the identity and order of the coding tags within extended recording tags.

Following sequencing of the nucleic acid libraries (e.g., of extended nucleic acids), the resulting sequences can be collapsed by their UMIs if used and then associated to their corresponding polypeptides and aligned to the totality of the proteome. Resulting sequences can also be collapsed by their compartment tags and associated to their corresponding compartmental proteome, which in a particular embodiment contains only a single or a very limited number of protein molecules. Both protein identification and quantification can easily be derived from this digital peptide information.

The methods disclosed herein can be used for analysis, including detection, quantitation and/or sequencing, of a plurality of polypeptides simultaneously (multiplexing). Multiplexing as used herein refers to analysis of a plurality of polypeptides in the same assay. The plurality of polypeptides can be derived from the same sample or different samples. The plurality of polypeptides can be derived from the same subject or different subjects. The plurality of polypeptides that are analyzed can be different polypeptides, or the same polypeptide derived from different samples. A plurality of polypeptides includes 2 or more polypeptides, 5 or more polypeptides, 10 or more polypeptides, 50 or more polypeptides, 100 or more polypeptides, 500 or more polypeptides, 1,000 or more polypeptides, 5,000 or more polypeptides, 10,000 or more polypeptides, 50,000 or more polypeptides, 100,000 or more polypeptides, 500,000 or more polypeptides, or 1,000,000 or more polypeptides.

Provided herein are also kits and articles of manufacture comprising components for preforming tethering reactions and complex formation between polypeptides to be analyzed or sequenced, couplers, binding agents and stabilizing components. In some embodiments, the kits further contain other reagents for treating and analyzing polypeptides. The kits and articles of manufacture may include any one or more of the reagents and components used in the methods described in the present disclosure. In some embodiments, the kit comprises reagents for preparing samples for preforming the binding or sequencing reactions, such as for preparing polypeptides from a sample and joining with stabilizing components.

In some embodiments, the kit comprises a coupler, wherein the coupler is configured to bind to a terminal amino acid of the polypeptide to form a coupler-polypeptide complex; a reagent for cleaving the peptide bond between the terminal amino acid and the penultimate terminal amino acid of the polypeptide within the coupler-polypeptide complex to generate a coupler-amino acid complex; and a binding agent capable of binding to the coupler-amino acid complex. In some embodiments, the kit further comprises a coding tag with identifying information regarding the binding agent and configured to be associated with the binding agent, and/or a recording tag configured to be associated with the polypeptide. In some embodiments, the binding agent capable of binding to the coupler-amino acid complex is configured to bind specifically to the amino acid from the coupler-amino acid complex. In some embodiments, the coding tag or the recording tag comprises a unique molecular identifier (UMI) or a barcode sequence. In some embodiments, the kit further comprises a solid support comprising a functionalized surface, wherein the polypeptide or an associated recording tag is configured to be attached to the solid support either directly or via a linker. In some embodiments, the kit further comprises components of first or second stabilizing components and a linking agent. In some embodiments, the kit further comprises a terminal modifier agent configured to modify the terminal amino acid of the polypeptide to produce a modified terminal amino acid of the polypeptide, wherein the coupler is configured to bind to the modified terminal amino acid of the polypeptide.

In some embodiments, the reagent for cleaving the peptide bond comprises a modified dipeptidyl peptidase comprising an unmodified dipeptidyl peptidase comprising at least one mutation in a substrate binding site, wherein (i) the unmodified dipeptidyl peptidase removes two terminal amino acids from a polypeptide; and (ii) the modified dipeptidyl peptidase is configured to cleave the coupler-amino acid complex from the polypeptide. In some embodiments, the reagent for cleaving the peptide bond comprises a set of dipeptidyl peptidase enzymes, comprising at least two different modified dipeptidyl peptidases, wherein: (i) each of the modified dipeptidyl peptidases from the set of dipeptidyl peptidase enzymes is configured to cleave the coupler-amino acid complex from the polypeptide, and comprises an unmodified dipeptidyl peptidase comprising at least one mutation in a substrate binding site; (ii) the unmodified dipeptidyl peptidase is configured to remove two terminal amino acids from the polypeptide; and (iii) the modified dipeptidyl peptidases from the set of dipeptidyl peptidase enzymes have different specificities for the amino acids from the coupler-amino acid complexes, which the modified dipeptidyl peptidases are configured to cleave from the polypeptide. In some embodiments, the reagent for cleaving the peptide bond comprises a set of 2, 3, 4, 5, 6, 7, 8, 9, 10 or more dipeptidyl peptidase enzymes, each having different specificities for the amino acids from the coupler-amino acid complexes, which the modified dipeptidyl peptidases are configured to cleave from the polypeptide. For example, one dipeptidyl peptidase enzyme can recognize and cleave from the polypeptide preferentially small hydrophobic terminal amino acid residues in the complex with the coupler. Another dipeptidyl peptidase enzyme can recognize and cleave from the polypeptide preferentially bulky aromatic terminal amino acid residues in the complex with the coupler. Yet another dipeptidyl peptidase enzyme can recognize and cleave from the polypeptide preferentially charged terminal amino acid residues in the complex with the coupler. Yet another dipeptidyl peptidase enzyme can recognize and cleave from the polypeptide preferentially polar terminal amino acid residues in the complex with the coupler. Taken as a mixture, the described exemplary dipeptidyl peptidase enzymes will form a set dipeptidyl peptidase enzymes that will recognize and cleave from the polypeptide multiple different terminal amino acid residues in the complex with the coupler.

In some embodiments, the kit comprises a plurality of binding agents wherein each binding agent is associated with a unique coding tag. In some embodiments, the kit comprises a coupler configured to bind to a terminal amino acid of the polypeptide. In some embodiments, the coupler comprises or is associated with a stabilizing component configured to form a tethering complex with a second stabilizing component associated with a polypeptide to be analyzed. In some embodiments, the kit comprises a reagent for cleaving the peptide bond between the terminal amino acid and the penultimate terminal amino acid of the polypeptide within the coupler-polypeptide complex to generate a coupler-amino acid complex. In some embodiments, the reagent for cleaving the peptide bond comprises a modified dipeptidyl peptidase comprising an unmodified dipeptidyl peptidase comprising at least one mutation in a substrate binding site, wherein (i) the unmodified dipeptidyl peptidase removes two terminal amino acids from a polypeptide; and (ii) the modified dipeptidyl peptidase is configured to cleave the coupler-amino acid complex from the polypeptide. In some aspects, the kits contain components for identifying a terminal amino acid of a polypeptide, comprising the steps of: (a) providing a polypeptide and an associated recording tag attached to a solid support; (b) contacting the polypeptide with a coupler, wherein the coupler binds to a terminal amino acid of the polypeptide to form a coupler-polypeptide complex; (c) attaching the coupler-polypeptide complex to the solid support; (d) cleaving the coupler-polypeptide complex from the polypeptide, thereby providing a coupler-amino acid complex attached to the solid support; (e) contacting the coupler-amino acid complex with a binding agent capable of binding to the coupler-amino acid complex, wherein the binding agent comprises a coding tag with identifying information regarding the binding agent; (f) transferring the information of the coding tag of the binding agent to the recording tag; (g) analyzing the recording tag extended after information transfer, thereby identifying the terminal amino acid of the polypeptide. In some embodiments, the kits optionally include instructions for performing the tethering reaction.

In some embodiments, the kits comprise one or more of the following components: binding agent(s), stabilizing component(s), linking agent(s), solid support(s), recording tag(s), reagent(s) for attaching the stabilizing components, reagent(s) for transferring information, sequencing reagent(s), and/or any reagents as described in the methods for performing the tethering reaction and analyzing polypeptides (e.g., proteins, polypeptides, or peptides), enzyme(s), buffer(s), etc.

In some embodiments, the kits also include other components for treating the polypeptides (e.g., proteins, polypeptides, or peptides), preforming a tethering reaction, and analysis of the same including other reagent(s) for analysis of the polypeptide. In one aspect, provided herein are components used to prepare a reaction mixture. In preferred embodiments, the reaction mixture is a solution. In preferred embodiments, the reaction mixture includes one or more of the following: stabilizing component(s), linking agent(s), solid support(s), recording tag(s), reagent(s) for attaching or associating the stabilizing components, reagent(s) for transferring information, sequencing reagent(s), binding agent(s) with associated stabilizing component(s) and/or coding tag(s), buffer(s).

In another aspect, disclosed herein is a kit for performing a tethering reaction comprising a library of binding agents, wherein each binding agent comprises or is associated with one or more stabilizing components, and a coding tag comprising identifying information regarding the binding moiety. In some embodiments, the binding moiety is capable of binding to one or more N-terminal, internal, or C-terminal amino acids of the polypeptide, or capable of binding to the one or more N-terminal, internal, or C-terminal amino acids of a peptide modified by a functionalizing reagent. In some cases, the kit also includes linking agents, wherein the linking agent comprises a chemical reagent, a non-biological reagent, a biological reagent, or a combination thereof. In some cases, the linking agent comprises a polypeptide or protein. In some cases, the linking agent comprises a metal ion.

In some embodiments, the kit further comprises reagents for treating the polypeptides. Any combination of fractionation, enrichment, and subtraction methods, of the proteins or polypeptides may be performed. For example, the reagent may be used to fragment or digest the proteins. In some cases, the kit comprises reagents and components to fractionate, isolate, subtract, enrich proteins. In some embodiments, the kits further comprises a protease such as trypsin, LysN, or LysC. In some embodiments, the kit comprises a support for immobilizing the one or more polypeptides and reagents for immobilizing the polypeptide on a support.

In some embodiments, the kit also comprises one or more buffers or reaction fluids necessary for any of the tethering and binding reactions to occur. Buffers including wash buffers, reaction buffers, and binding buffers, elution buffers and the like are known to those or ordinary skill in the arts. In some embodiments, the kits further include buffers and other components to accompany other reagents described herein. The reagents, buffers, and other components may be provided in vials (such as sealed vials), vessels, ampules, bottles, jars, flexible packaging (e.g., sealed Mylar or plastic bags), and the like. Any of the components of the kits may be sterilized and/or sealed.

In some embodiments, the kit includes one or more reagents for nucleic acid sequence analysis. In some embodiments, the reagent for sequence analysis is for use in sequencing by synthesis, sequencing by ligation, single molecule sequencing, single molecule fluorescent sequencing, sequencing by hybridization, polony sequencing, ion semiconductor sequencing, pyrosequencing, single molecule real-time sequencing, nanopore-based sequencing, or direct imaging of DNA using advanced microscopy, or any combination thereof.

In addition to above-mentioned components, the subject kits may further include instructions for using the components of the kit to practice the subject methods, i.e., instructions for sample preparation, treatment and/or analysis. The kits described herein may also include other materials desirable from a commercial and user standpoint, including other buffers, diluents, filters, syringes, and package inserts with instructions for performing any methods described herein.

Any of the above-mentioned kit components, and any molecule, molecular complex or conjugate, reagent (e.g., chemical or biological reagents), agent, structure (e.g., support, surface, particle, or bead), reaction intermediate, reaction product, binding complex, or any other article of manufacture disclosed and/or used in the exemplary kits and methods, may be provided separately or in any suitable combination in order to form a kit.

### Exemplary Embodiments

Among the provided embodiments are:
1. A method for identifying a terminal amino acid of a polypeptide, comprising the steps of:
(a) providing a polypeptide and an associated recording tag attached to a solid support;
(b) contacting the polypeptide with a coupler, wherein the coupler binds to a terminal amino acid of the polypeptide to form a coupler-polypeptide complex;
(c) attaching the coupler to the solid support;
(d) cleaving the peptide bond between the terminal amino acid and the penultimate terminal amino acid of the polypeptide within the coupler-polypeptide complex to generate a coupler-amino acid complex attached to the solid support;
(e) contacting the coupler-amino acid complex with a binding agent capable of binding selectively to the coupler-amino acid complex, wherein the binding agent comprises a coding tag with identifying information regarding the binding agent;
(f) transferring the information of the coding tag of the binding agent to the recording tag to generate an extended recording tag; and
(g) analyzing the extended recording tag, thereby identifying the terminal amino acid of the polypeptide.
2. The method of embodiment 1, wherein the terminal amino acid is modified to produce a modified terminal amino acid before contacting the polypeptide with the coupler; at step (b) the coupler binds to the modified terminal amino acid; at step (d) a coupler-modified amino acid complex attached to the solid support is generated after cleavage of the peptide bond between the terminal amino acid and the penultimate terminal amino acid of the polypeptide; and at step (e) the binding agent is capable of binding to the coupler-modified amino acid complex.
3. The method of embodiment 1 or 2, wherein the coupler is releasably attached to the solid support.
4. The method of any one of embodiments 1-3, wherein the polypeptide is further associated with a first stabilizing component; the coupler comprises a second stabilizing component; at step (c) after binding of the coupler to the terminal amino acid of the polypeptide, the first and second stabilizing components are linked together to form a tethering complex that comprises a first stabilizing component attached to the solid support and the second stabilizing component linked to the coupler-polypeptide complex; and at step (d) the coupler-amino acid complex generated after cleavage of the peptide bond between the terminal amino acid and the penultimate terminal amino acid of the polypeptide is releasably attached to the solid support via the tethering complex.
5. The method of embodiment 4, wherein the first stabilizing component is releasably associated with the polypeptide attached to the solid support.
6. The method of embodiment 4 or embodiment 5, wherein the stabilizing components are linked upon introduction of a linking agent that binds to the first stabilizing component and to the second stabilizing component.
7. The method of embodiment 6, wherein the linking agent is releasably associated with the first stabilizing component.
8. The method of embodiment 6, wherein the linking agent activates a stabilizing component.
9. The method of embodiment 6, wherein the linking agent comprises a linking polypeptide that binds to the first stabilizing component and to the second stabilizing component.
10. The method of embodiment 6, wherein the first or second stabilizing component comprises a polynucleotide; the stabilizing components are linked upon introduction of a linking agent that hybridizes to the polynucleotide of one of the stabilizing components.
11. The method of any one of embodiments 4-10, wherein the first stabilizing component is the same as the second stabilizing component.
12. The method of any one of embodiments 6-10, wherein the stabilizing components are linked upon introduction of a linking agent, the linking agent binds to the first stabilizing component and to the second stabilizing component, and the first stabilizing component has a lower affinity to the linking agent in comparison to an affinity of the second stabilizing component to the linking agent.
13. The method of any one of embodiments 1-12, wherein (a) the binding agent binds to the coupler-amino acid complex, but does not specifically bind to the corresponding amino acid separated from the coupler-amino acid complex, or affinity of the binding agent for the corresponding amino acid separated from the coupler-amino acid complex is reduced compared to affinity of the binding agent for the coupler-amino acid complex by at least an order of magnitude; or
   (b) the binding agent comprises an engineered carboxypeptidase.
14. The method of any one of embodiments 1-13, wherein the extended recording tag after information transfer is analyzed using a nucleic acid sequencing method.
15. The method of any one of embodiments 1-14, wherein transferring the information of the coding tag comprises contacting the coding tag with a reagent for transferring information, the reagent comprising a reagent for primer extension reaction, a chemical ligation reagent or a biological ligation reagent.
16. The method of any one of embodiments 1-15, wherein the coupler binds to an N-terminal amino acid (NTAA) of the polypeptide.
17. The method of any one of embodiments 1-16, which further comprises releasing the coupler-amino acid complex from the solid support after transferring the information of the coding tag to the recording tag.
18. The method of any one of embodiments 1-17, wherein the coupler-amino acid complex is cleaved from the polypeptide by a Cleavase comprising an unmodified exopeptidase comprising at least one mutation in a substrate binding site, wherein (i) the unmodified exopeptidase removes two terminal amino acids from a polypeptide; and (ii) the Cleavase is configured to cleave the coupler-amino acid complex from the polypeptide.
19. A method for identifying at least a portion of a sequence of a polypeptide, comprising the steps of:
(a) providing a polypeptide and an associated recording tag attached to a solid support;
(b) contacting the polypeptide with a coupler, wherein the coupler binds to a terminal amino acid of the polypeptide to form a coupler-polypeptide complex;
(c) attaching the coupler to the solid support;
(d) cleaving the peptide bond between the terminal amino acid and the penultimate terminal amino acid of the polypeptide within the coupler-polypeptide complex, thereby exposing a new terminal amino acid of the polypeptide and generating a coupler-amino acid complex attached to the solid support;
(e) contacting the coupler-amino acid complex attached to the solid support with a binding agent capable of binding to the coupler-amino acid complex, wherein the binding agent comprises a coding tag with identifying information regarding the binding agent;
(f) transferring the information of the coding tag of the binding agent to the recording tag to generate an extended recording tag;
(g) releasing the coupler-amino acid complex from the solid support;
(h) repeating steps (b) through (g) at least one more time; and
(i) analyzing the extended recording tag after information transfer, thereby identifying at least a portion of the sequence of the polypeptide.
20. The method of embodiment 19, wherein the terminal amino acid is modified to produce a modified terminal amino acid before contacting the polypeptide with the coupler; at step (b) the coupler binds to the modified terminal amino acid; at step (d) a coupler-modified amino acid complex attached to the solid support is generated after cleavage of the peptide bond between the terminal amino acid and the penultimate terminal amino acid of the polypeptide; and at step (e) the binding agent is capable of binding to the coupler-modified amino acid complex.
21. The method of embodiment 19 or 20, wherein the coupler is releasably attached to the solid support.
22. The method of any one of embodiments 19-21, wherein the polypeptide is further associated with a first stabilizing component and the coupler comprises a second stabilizing component; at step (c) after binding of the coupler to the terminal amino acid of the polypeptide, the first and second stabilizing components are linked together to form a tethering complex that comprises a first stabilizing component attached to the solid support and the second stabilizing component linked to the coupler-polypeptide complex; and at step (d) the coupler-amino acid complex generated after cleavage of the peptide bond between the terminal amino acid and the penultimate terminal amino acid of the polypeptide is releasably attached to the solid support via the tethering complex.
23. The method of embodiment 22, wherein the first stabilizing component is releasably associated with the polypeptide attached to the solid support.
24. The method of embodiment 22 or embodiment 23, wherein the stabilizing components are linked upon introduction of a linking agent that binds to the first stabilizing component and to the second stabilizing component.
25. The method of embodiment 24, wherein the linking agent is releasably associated with the first stabilizing component.
26. The method of embodiment 24, wherein the linking agent activates a stabilizing component.
27. The method of embodiment 24, wherein the linking agent comprises a linking polypeptide that binds to the first stabilizing component and to the second stabilizing component.
28. The method of any one of embodiments 22-27, wherein the first or second stabilizing component comprises a polynucleotide; the stabilizing components are linked upon introduction of a linking agent that hybridizes to the polynucleotide of one of the stabilizing components.
29. The method of any one of embodiments 22-28, wherein the first stabilizing component is the same as the second stabilizing component.
30. The method of any one of embodiments 22-28, wherein the stabilizing components are linked upon introduction of a linking agent, the linking agent binds to the first stabilizing component and to the second stabilizing component, and the first stabilizing component has a lower affinity to the linking agent in comparison to an affinity of the second stabilizing component to the linking agent.
31. The method of any one of embodiments 19-30, wherein (a) the binding agent binds to the coupler-amino acid complex, but does not bind to the corresponding amino acid separated from the coupler-amino acid complex, or affinity of the binding agent for the corresponding amino acid separated from the coupler-amino acid complex is reduced compared to affinity of the binding agent for the coupler-amino acid complex by at least an order of magnitude; or
   (b) the binding agent comprises an engineered carboxypeptidase.
32. The method of any one of embodiments 19-31, wherein the extended recording tag after information transfer is analyzed using a nucleic acid sequencing method.
33. The method of any one of embodiments 19-32, wherein transferring the information of the coding tag comprises contacting the coding tag with a reagent for transferring information, the reagent comprising a reagent for primer extension reaction, a chemical ligation reagent or a biological ligation reagent.
34. The method of any one of embodiments 19-33, wherein the coupler binds to a N-terminal amino acid (NTAA) of the polypeptide.
35. The method of any one of embodiments 19-34, which further comprises releasing the coupler-amino acid complex from the solid support after transferring the information of the coding tag to the recording tag.
36. The method of any one of embodiments 19-35, wherein the peptide bond between the terminal amino acid and the penultimate terminal amino acid of the polypeptide within the coupler-amino acid complex is cleaved from the polypeptide by a Cleavase comprising an unmodified exopeptidase comprising at least one mutation in a substrate binding site, wherein (i) the unmodified exopeptidase removes two terminal amino acids from a polypeptide; and (ii) the Cleavase is configured to cleave the coupler-amino acid complex from the polypeptide.
37. A kit for analyzing a polypeptide, comprising: a coupler, wherein the coupler is configured to bind to a terminal amino acid of the polypeptide to form a coupler-polypeptide complex; a reagent for cleaving the peptide bond between the terminal amino acid and the penultimate terminal amino acid of the polypeptide within the coupler-polypeptide complex to generate a coupler-amino acid complex; and a binding agent capable of binding to the coupler-amino acid complex.
38. The kit of embodiment 37, further comprising a coding tag with identifying information regarding the binding agent and configured to be associated with the binding agent, and/or a recording tag configured to be associated with the polypeptide.
39. The kit of embodiment 37 or embodiment 38, wherein the reagent for cleaving the peptide bond comprises a Cleavase comprising an unmodified exopeptidase comprising at least one mutation in a substrate binding site, wherein (i) the unmodified exopeptidase removes two terminal amino acids from a polypeptide; and (ii) the Cleavase is configured to cleave the coupler-amino acid complex from the polypeptide.
40. The kit of any one of embodiments 37-39, wherein the coding tag or the recording tag comprises a unique molecular identifier (UMI) or a barcode sequence.
41. The kit of any one of embodiments 37-40, further comprising a solid support comprising a functionalized surface, wherein the polypeptide or an associated recording tag is configured to be attached to the solid support either directly or via a linker.
42. The kit of any one of embodiments 37-41, wherein the binding agent capable of binding to the coupler-amino acid complex is configured to bind to a specific amino acid from the coupler-amino acid complex.
43. The kit of any one of embodiments 37-42, further comprising a terminal modifier agent configured to modify the terminal amino acid of the polypeptide to produce a modified terminal amino acid of the polypeptide, wherein the coupler is configured to bind to the modified terminal amino acid of the polypeptide.
44. The kit of any one of embodiments 37-43, wherein the terminal amino acid of the polypeptide is an N-terminal amino acid.
45. The kit of any one of embodiments 37-44, wherein the binding agent comprises an engineered carboxypeptidase.

### EXAMPLES

The following examples are offered to illustrate but not to limit the methods, compositions, and uses provided herein. Certain aspects of the present invention, including, but not limited to, embodiments for the ProteoCode™ polypeptide sequencing assay, information transfer between coding tags and recording tags, methods for attachment of polynucleotide-polypeptide conjugate to a support, methods of making polypeptide-polynucleotide conjugate, methods of generating barcodes, methods of generating specific binding agents recognizing an N-terminal amino acid, reagents and methods for modifying and/or removing an N-terminal amino acid from a polypeptide were disclosed in earlier published application US 20190145982 A1, US 20200348308 A1, US 20200348307 A1, WO 2020/223000, the contents of which are incorporated herein by reference in its entirety.

### Example 1. Tethering of the NTAA-Coupler Complex to the Solid Support Using Streptavidin

The peptide-DNA conjugates are initially immobilized onto Sepharose beads with sparsely spaced capture DNA molecules comprising the recording tag and the desthiobiotin (DSB) molecule as a first stabilizing component (FIG. 1A). The coupler comprising an amine-reactive group (e.g. an activated ester such as NHS or PNP group), an NTM group, a linker, and a biotin group, is reacted with NTAA residues of the polypeptide-polynucleotide (polypeptide-DNA) conjugates immobilized on Sepharose beads. After reacting the coupler to the NTAAs, the DNA-polypeptide conjugates on the beads are washed twice with a high salt PBS-T buffer (1.1 mM KH₂PO₄, 3 mM Na₂HPO₄, 500 mM NaCl, 0.1% Tween 20), which is a high salt PBS buffer supplemented with 0.1% Tween 20. The tethering step is initiated by adding 50 nM streptavidin or neutravidin (as a linking agent) in the high salt PBS buffer and incubating at 25° C. for 5 min. The streptavidin/neutravidin acts as a linking agent to connect the biotin of the coupler to DSB (first stabilizing component) on the capture DNA (FIG. 1C). The use of a lower-affinity biotin analogue such as desthiobiotin (DSB) enables easy removal of the complex after the downstream binding/encoding step by competing with excess of biotin (1 mM). After linking reaction between streptavidin and biotin/DSB, 1-2 washes with PBS-T are performed to remove excess of streptavidin.

Alternative to a pre-installed first stabilizing component on the capture DNA, the first stabilizing component can be installed on demand via hybridization of an oligonucleotide comprising a stabilizing component such as biotin, DSB, mTet, TCO, etc. (FIG. 2C). As such, a tight binding or covalent coupling between the stabilizing component and linking agent does not preclude easy removal of the complex since the DNA hybridization-based installation of the stabilizing component is easily reversed via “de-hybridization” or dissociation using heat or alkaline denaturing conditions. Using a biotin-streptavidin-biotin stabilizing component-linking complex, the first stabilizing component is installed on demand by hybridizing a biotinylated oligonucleotide to a cognate sequence located at the 5′ terminus of the hairpin capture DNA comprising a recording tag and immobilized on the surface (FIG. 2C). The hybridization sequence (25-50 nucleotides for natural DNA; 10-20 LNA nucleotides (locked nucleic acid residues; contain a methylene bridge connecting the 2′ oxygen and 4′ carbon in the ribose moiety) is designed to be stable under thermophilic Cleavase working conditions, such as reactions performed at 50-65° C. in a low salt buffer supplemented optionally with 1-10 mM divalent cations (e.g. Mg²⁺). A typical DNA 35-mer has a melting temperature of >70° C. in a low salt buffer supplemented with 5 mM MgCl₂. The presence of magnesium does not significantly affect the Cleavase activity, for the preferred S46 Cleavase embodiment, but the presence of magnesium significantly stabilizes the DNA hybrid.

Hybridization was performed by incubating 10-100 nM biotinylated oligonucleotide in the high-salt PBST with the polypeptide-polynucleotide conjugate for 10 minutes at 37° C. The conjugates were washed twice with high-salt PBST to remove excess oligo stabilizer. In a preferred embodiment, a short LNA sequence of 10-25 nucleotides comprising a biotin stabilizing component probe is used. Use of LNA raises the melting temperature (Tm) about 2-6° C. for each LNA base relative to a standard base (Jolly P, et al., Oligonucleotide-based systems: DNA, microRNAs, DNA/RNA aptamers. Essays Biochem. (2016), 60(1): 27-35). The biotin moiety can be attached at the 3′, 5′ or within the body of the LNA oligonucleotide. A 12-mer LNA oligonucleotide is sufficient to maintain hybrid stability. The LNA sequence GTGATATGTCCG (SEQ ID NO: 13) has a Tm of 76° C. whereas the corresponding DNA sequence has a Tm of 29° C.

### Example 2. Tethering of the NTAA-Coupler Complex Using Homobifunctional iEDDA Linkers

The coupler comprising an NHS or PFP group and also comprising an mTet moiety was incubated with NTAA residues of the polypeptide-polynucleotide conjugates immobilized by hybridization to Sepharose beads covered by capture DNA molecules comprising the recording tag and an mTet moiety as a first stabilizing component (FIG. 2A). This mTet stabilizing component, in the preferred embodiment, is installed via hybridization of an mTet-labeled oligonucleotide to the recording tag similar to the installation of the biotin oligo in Example 1. In a preferred embodiment, the mTet oligonucleotide is hybridized after the NTF step. The NTF step installs an NTM moiety on the N-terminal amino acid by exposing the peptide to an activated ester of the NTM (FIG. 2B). Typical conditions for NTM coupling using NHS or PFP activated ester couplers are as follows: immobilized DNA-peptide conjugate is incubated with a 20 mM solution of coupler NTM-NHS or coupler NTM-PFP in 50% acetonitrile/25% dimethylacetamide/25% MOPS buffer (pH 7.6) at 40° C. for 60 min. Excess reagent is removed and quenched by washing 3× with TBST (20 mM Tris-Cl, 150 mM NaCl, 0.10% Tween-20; pH 7.6) or similar primary amine containing buffer.

A preferred embodiment of the coupler NTM-NHS architecture utilizes mTet-(aliphatic linker or PEG linker)-NTM-NHS/PFP, and the NTM moiety is a small molecule capable of being synthesized as an activated NHS/PFP ester with an appendage for attachment of the PEG/aliphatic linker. For efficient downstream cleavage, the installed NTM and tethering group should be compatible with the Cleavase activity. After reacting the coupler to the NTAAs, the conjugates on the beads are washed 2-3× with high salt PBS-T buffer (1.1 mM KH₂PO₄, 3 mM Na₂HPO₄, 500 mM NaCl, 0.1% Tween 20). The first stabilizing component, mTet, is installed via hybridization of an mTET-labeled oligonucleotide to its complementary region on the capture/recording tag sequence (FIG. 2C). The hybridization is performed by incubating the conjugate on beads with 10 nM of mTet-labeled oligonucleotide in high salt PBS-T buffer supplemented with 10% formamide for 10-20 min at room temperature. After hybridization, the immobilized conjugates are washed once with high-salt PBS-T buffer and once with the PBS-T buffer. The tethering step is initiated by adding 1-100 uM TCO-PEG11-TCO linking agent in high salt PBS buffer and incubating at 25° C. for 5-20 min. TCO-PEG11-TCO acts as a linking agent to covalently connect mTet on the mTet oligonucleotide hybridized to the capture DNA to the mTet linked to the NTM-NTAA of the peptide (FIG. 2D). After completion of tethering, the tethered complex is digested with a Cleavase (FIG. 2E, the tethered NTM-NTAA complex is released from the peptide yet remains tethered to the capture/recording tag and is available for binding/encoding detection (FIG. 2F).

### Example 3. Tethering of the NTAA-Coupler Complex Using DNA Hybridization

The coupler comprising an NTM group and oligo A is incubated with NTAA residues of the polypeptide-polynucleotide conjugates immobilized by hybridization to Sepharose beads covered by capture DNA molecules comprising the recording tag and the oligo B molecule as a first stabilizing component. After binding of the coupler to the NTAA of the immobilized polypeptide, a splint DNA is added as a linking agent that is able to hybridize to the oligo A linked to the polypeptide and the oligo B on the 5′ of the capture hairpin DNA; the hybridization reaction occurs in the ligation solution comprising 50 mM Tris-HCl, pH 7.5, 10 mM MgCl₂, 1 mM ATP, 10 mM DTT and 0.5 U/uL T4 DNA ligase. The coupler-polypeptide is attached with the corresponding capture hairpin DNA by covalent linkage between the oligo A and the oligo B after incubation at 25° C. for 30 min. After cleavage and encoding, the hybridization complex can be removed by nicking endonuclease digestion and the oligo B can be regenerated on the 5′ end of the capture hairpin DNA. The covalent linkage can include reversible photochemical ligation using 5-carboxyvinyldeoxyuridine (cvU).

### Example 4. Coupler Reagent for NTAA Labeling and Cleavage

In this Example, the tethering and release of the tethered NTAA is facilitated by a chemical method. Labeling NTAA of an immobilized peptide is performed under mild conditions according to the methods disclosed in U.S. patent application Ser. No. 17/606,759 filed on Oct. 26, 2021. Briefly, immobilized peptide conjugated to the recording DNA tag and containing free NTAA is incubated with 30 mM PMI (pyrazole methanimine) in a 3:2 DMAc:MOPS buffer at pH=7.6 for 0.5 h at 40° C., and as a result, the NTAA is functionalized with a guanidinyl moiety (see FIG. 4). Once functionalized, the tethering component affixed on one end to a hydrazine in 1:1 DMSO:PBS buffer at pH=7.4 displaces the pyrazole to form an aminoguanidine at the NTAA, bearing the linked tether. To remove the NTAA from the rest of the peptide, the peptidyl aminoguanidine tether is treated with 1M ammonium phosphate (aq; pH 6.5) for 0.5-1 h at 90° C. This releases the NTAA in the form of a tethered 2-amino imidazolone that can be subsequently recognized by a NTAA-specific binding agent, followed by performing the encoding reaction (see Example 5).

### Example 5. Exemplary Embodiment of Methods for Identifying a Polypeptide Analyte

Polypeptide analytes from a human sample are immobilized on a porous bead (sepharose bead), and each polypeptide analyte is associated with an individual nucleic acid recording tag. To identify polypeptide analytes in a high throughput manner (500 or more peptides in parallel), the following steps are taken. The NTAA residues of the immobilized polypeptide analytes are contacted with a coupler (see, for example, the coupler reagent from Example 4), wherein the coupler binds to the NTAA residues of the polypeptides to form a coupler-NTAA complex. Then, the coupler is attached to surface of the bead via a tether, followed by induced cleavage of the coupler-NTAA complex from the polypeptide analytes (see the cleavage reaction in FIG. 4 and Example 4), generating isolated amino acid-coupler complexes. Then, binding agents are used to recognize the isolated amino acid-coupler complexes, wherein each binding agent comprises a coding tag with identifying information regarding the binding agent.

The described generation of isolated amino acid-coupler complexes simplify development of binding agents that capable of specific binding to coupler-amino acid complexes. Several examples of binding agents that specifically recognize NTAA residues of immobilized peptides have been published (see, e.g., U.S. Pat. No. 9,435,810 B2, U.S. Ser. No. 10/481,162 B2, US 2019/0145982 A1, U.S. patent application Ser. Nos. 17/539,033 and 17/727,677, incorporated herein), evolved from various scaffolds, including ClpS, amino acyl tRNA synthetases, anticalins, carbonic anhydrases and others. When coupler-amino acid complexes are attached to the polypeptide backbone (before cleavage), adjacent amino acid residues of the polypeptide interfere with ability of binding agents to specifically recognize terminal amino acid residues. Isolating (separating) amino acid-coupler complexes away from the “remainings” of peptide analytes resolves this problem and allows for generation of binding agents with a higher affinity towards a modified NTAA residue (NTAA residue in complex with the coupler).

Next, following binding of binding agents to the isolated amino acid-coupler complexes, information regarding each of binding agent's identity is transferred to the corresponding nucleic acid recording tag associated with each polypeptide analyte. After that, the coupler-amino acid complex is released from the solid support, and the steps of contacting the polypeptide analyte with the coupler, attaching the coupler to the solid support, cleaving the peptide bond between the terminal amino acid and the penultimate terminal amino acid of the polypeptide within the coupler-polypeptide complex, contacting the coupler-amino acid complex attached to the solid support with a binding agent, transferring the information of the coding tag of the binding agent to the recording tag, and releasing the coupler-amino acid complex from the solid support are repeated at least one more time, and preferably several times. Each cycle further extends the recording tag associated with the polypeptide analyte, building up a final extended recording tag that accumulates identifying information about all binding events for the polypeptide analyte (the whole binding history of the polypeptide analyte).

After transfer of coding tag information to the extended recording tag in one or more cycles, the final extended recording tag is analyzed by a nucleic acid sequencing. Preferably, multiple polypeptide analyte are analyzed in a single assay, so for each polypeptide analyte, an independent associated final extended recording tag is generated. Final extended recording tags from multiple polypeptides are amplified using a universal primer incorporated in the recording tag, and, optionally, utilizing adaptors specific for chosen sequencing system. Multiple final extended recording tags are sequenced by one of the available NGS platforms, and the sequences of the final extended recording tags are processed to extract information regarding identities and order of the binding agents that were bound to NTAA-coupler complexes during the described encoding cycles. When a binding agent is not selective for a particular amino acid residue in complex with the coupler, but instead can recognize any amino acid residue from a group of amino acid residues in complex with the coupler, probabilities of having particular amino acid residues in a current position of the polypeptide analyte are calculated based on known specificity and selectivity of the binding agent. After calculating these probabilities for every binding agent that are encoded in the final extended recording tag, a pattern is created for each analyzed polypeptide analyte. Then, genomic or proteomic database containing information regarding proteins potentially present in the analyzed sample can be used to create in silico patterns for the whole proteome (or part of the proteome) indicative of the presence and order of amino acid residues at certain places. Information extracted from final extended recording tags can be matched with these patterns in order to identify the immobilized polypeptide with certain probability. Even in the presence of errors that can occur during the described encoding cycles (such as failure to transfer the coding tag information), only limited number of specific amino acid residues is sufficient to identify the immobilized polypeptide with high probability given that both type and order of the specific amino acid residues can be obtained during the analysis of the extended recording tag. Exemplary calculations of minimal number of the specific amino acid residues required to achieve a certain probability in polypeptide identification for a particular proteome were previously published (Swaminathan J, et al., A theoretical justification for single molecule peptide sequencing. PLoS Comput Biol. 2015 Feb. 25; 11(2):e1004080).

### Example 6. Selection and Design of Binding Agents Capable of Specific Binding to the Coupler-Amino Acid Complex

A tethered coupler-amino acid complex consists of a tether moiety (hereafter termed T₁) harboring the C-terminal amino acid side-chain (hereafter termed R₁), collectively referred to as T₁R₁ (e.g., a tethered coupler-amino acid complex harboring an alanine sidechain is referred to as T₁A, the tether moiety harboring an isoleucine side-chain is referred to as T₁I, and so on—those skilled in the art would recognize that there are 20 possible T₁R₁ moieties using the 20 canonical amino acids, and dozens more using post-translational modifications of the 20 canonical amino acids). The T₁R₁ moiety can be bound by an appropriate binding agent configured to bind selectively or semi-selectively to the tethered coupler-amino acid complex. The binding agent can be designed to bind selectively to T₁R₁ moieties using state-of-the-art computational protein design methodologies described below. First, in order to implement computational protein design methodologies, the T₁R₁ moieties must be appropriately parameterized by calculating the atomic partial charges and conformational degrees of freedom, which is accomplished using the Rosetta macromolecular modeling and design software suite (Alford, Rebecca F et al. “The Rosetta All-Atom Energy Function for Macromolecular Modeling and Design.” Journal of chemical theory and computation vol. 13, 6 (2017): 3031-3048).

In one embodiment of this disclosure, after each T₁R₁ moiety is appropriately parameterized, each T₁R₁ moiety can be docked into suitable protein-based scaffolds using the RosettaLigand protocol (Lemmon, Gordon, and Jens Meiler. “Rosetta Ligand docking with flexible XML protocols.” Methods in molecular biology (Clifton, N.J.) vol. 819 (2012): 143-55) of the Rosetta macromolecular modeling and design software suite (see Alford, Rebecca F et al, 2017 above). The RosettaLigand protocol effectively samples rigid body positions and orientations of each T₁R₁ moiety, as well as the conformational degrees of freedom of each T₁R₁ moiety, using Monte Carlo-based minimization followed by evaluation of the sampled conformations with a full-atom energy function that includes van der Walls interactions, an electrostatic potential, a hydrogen bonding potential, and an implicit solvation potential. For the lowest energy docked conformations of each T₁R₁ moiety into each suitable protein-based scaffold, protein scaffold sequence design of residues with a C_{α} atom within a 6.0 Å distance of any atom in the T₁R₁ moiety can be undertaken by iterating between fixed-backbone sampling of designable residue sidechains with repacking of sidechains (Bhardwaj, Gaurav et al. “Accurate de novo design of hyperstable constrained peptides.” Nature vol. 538, 7625 (2016): 329-335) of neighboring residues with a C_{α} atom within a 6.0 Å distance from the C_{α} atoms of any designable residues, and flexible backbone, sidechain, and T₁R₁ moiety energy minimization for the rest of protein-tethered coupler-amino acid complex, minimizing the total energy of the system. The overarching goal of protein-based scaffold sequence design is to stabilize the T₁R₁ conformation obtained from RosettaLigand docking, optimize protein-T₁R₁ conformer interaction energy, and gamer T₁R₁ binding specificity in each protein-based scaffold.

In another embodiment of this disclosure, after each T₁R₁ moiety is parameterized, for each T₁R₁ moiety the conformers with the lowest internal energy can be selected after scoring each conformer with a Universal Force Field (UFF) (see A. K. Rappe, et al., UFF, a full periodic table force field for molecular mechanics and molecular dynamics simulations. J. Am. Chem. Soc. 1992, 114, 25, 10024-10035) or one of the Merck Molecular Force Field (MMFF) variants (Tosco, P., Stiefl, N. & Landrum, G. Bringing the MMFF force field to the RDKit: implementation and validation. J Cheminform 6, 37 (2014)) MMFF94 or MMFF94s implemented with the chemoinformatics library RDKit (Wang, S.; et al., Improving Conformer Generation for Small Rings and Macrocycles Based on Distance Geometry and Experimental Torsional-Angle Preferences. J. Chem. Inf. Model. 2020, 60(4), 2044-2058). For the lowest energy conformers for each T₁R₁ moiety, novel protein sidechain interactions with the T₁R₁ conformers can be generated, and the T₁R₁ conformers docked into suitable protein-based scaffolds, using the Rotamer Interaction Field (RIF) docking method (Dou J, et al., De novo design of a fluorescence-activating β-barrel. Nature. 2018; 561(7724):485-491). The RIF docking algorithm generates an ensemble of billions of discrete amino acid sidechain placements around the target T₁R₁ moiety that form favorable intermolecular interactions, including hydrogen bonds and hydrophobic interactions. Subsequently, suitable protein-based scaffolds are docked into this pre-generated interaction ensemble using a grid-based hierarchical search algorithm (see Dou J, et al., 2018 above). For each T₁R₁ conformer successfully RIF docked into each suitable protein-based scaffold, protein scaffold sequence design of residues with a C_{α} atom within a 6.0 Å distance of any atom in the T₁R₁ conformer can be undertaken after constraining the coordinates of the T₁R₁ conformer and sidechains obtained via RIF docking. A sequence design protocol may iterate between fixed-backbone design of designable residues around the T₁R₁ conformer to optimize T₁R₁ conformer-protein interaction energy, and flexible-backbone and sidechain minimization for the rest of protein, ultimately minimizing the total energy of the system. The goal of sequence design is to stabilize the T₁R₁ conformer, as well as the sidechain conformations obtained from RIF docking, to pre-organize the T₁R₁ binding pocket for T₁R₁ specificity.

In another embodiment, using neural network-based de novo protein design, novel protein folds are hallucinated around the ensemble of discrete amino acid sidechain placements around T₁R₁ conformers obtained from RIF docking using an inpainting methodology for designing scaffolding proteins around functional sites (J. Wang, S. et al., Deep learning methods for designing proteins scaffolding functional sites. bioRxiv (2021)).

In each computational protein design methodology described herein, the resulting designed protein-based binding agent can confer binding specificity for T₁R₁ moieties, enabling specific binding to the tethered coupler-amino acid complex. Suitable protein-based scaffolds for the RosettaLigand and RIF docking methodologies include the metallocarboxypeptidase enzymes Carboxypeptidase A (CPA), Carboxypeptidase B (CPB), and Carboxypeptidase T (CPT), such as the thermophilic bacterial carboxypeptidase from Thermoactinomyces vulgaris. For example, in the protein-based CPT scaffold, positions that are known to confer substrate specificity can be selected for redesign, including D260 and T262 residues (Grishin A M, et al., Structural principles of the broad substrate specificity of Thermoactinomyces vulgaris carboxypeptidase T-role of amino acid residues at positions 260 and 262. Protein Eng Des Sel. 2008; 21(9):545-551), as well as L211, L254, D263, E277 residues and residues in the P248-D258 loop (Akparov, V. K., et al., Structural insights into the broad substrate specificity of carboxypeptidase T from Thermoactinomyces vulgaris. (2015) FEBS J, 282: 1214-1224). Suitable protein-based scaffolds are also not limited to naturally existing proteins. De novo designed proteins constitute a growing class of hyperstable proteins with novel folds and pocket shapes suitable for binding T₁R₁ conformers, including TIM barrels (Huang, P S., et al., De novo design of a four-fold symmetric TIM-barrel protein with atomic-level accuracy. Nat Chem Biol 12, 29-34 (2016)), beta-barrels (Dou J, et al., De novo design of a fluorescence-activating β-barrel. Nature. 2018; 561(7724):485-491), and helical bundles (Jooyoung Park, et al., De novo design of a homo-trimeric amantadine-binding protein; eLife 8:e47839 (2019)). For the neural network-based de novo protein design, novel protein folds previously uncharacterized in the Structural Classification of Proteins (SCOP) database (Antonina Andreeva, et al., The SCOP database in 2020: expanded classification of representative family and superfamily domains of known protein structures, Nucleic Acids Research, Volume 48, Issue D1, 2020, pages D376-D382) can also be generated to serve as suitable protein-based scaffolds for specific binding to the tethered coupler-amino acid complex.

As particular examples of engineering binding agents, we computationally analyzed the crystal structures of wild-type metallocarboxypeptidase enzymes in complex with inhibitor molecules, where the inhibitor molecules contain moieties with structural similarity to the R₁ moieties previously discussed. To prepare structural models for computational analysis, Protein Data Bank (PDB) accession codes 1CBX, 7CPA, 1ZG8, 1ZG9, 1H8L, 3V7Z, 4DUK, and 3D67 were downloaded, the chain A protein structure was extracted, the Zn²⁺ ion bound in the pocket of the chain A protein structure was renumbered to chain A at the next available residue position, the inhibitor molecule juxtaposing the Zn²⁺ ion and bound in the pocket of the chain A protein structure was renumbered to chain A at the next available residue position, and all other ligands and water molecules in the structures were removed. Using PyRosetta macromolecular modeling and design software (Chaudhury S, et al., PyRosetta: a script-based interface for implementing molecular modeling algorithms using Rosetta. Bioinformatics. 2010; 26(5):689-691), we computationally analyzed the prepared structural models to select residue positions for rationally designed mutations in the metallocarboxypeptidase enzymes to achieve specific binding to the aforementioned tethered coupler-amino acid complex. For the inhibitor molecule in each prepared structure, we manually selected the heavy (non-hydrogen) atoms bound in the protein pocket but not directly interacting with the bound Zn²⁺ ion in the pocket, and for each selected heavy atom we computationally selected residues in the metallocarboxypeptidase enzyme which have a C_{β} atom within an 8.0 Å sphere of the selected heavy atom, and computed the union of these residue sets forming a single set (i.e., residue set A). Additionally, residues in each metallocarboxypeptidase enzyme with any atom within a 4.5 Å distance from any atom in the bound inhibitor molecule were selected (i.e., residue set B). Finally, residues not participating in chelation of the bound Zn²⁺ ion in the pocket were selected (i.e., residue set C). To select residue positions for rationally designed mutations, we took the intersection between the three residue sets (i.e., A∩B∩C) as reported in the “Residues” column of Table 1. Additionally, for each structure, the distance between the C_{α} atom of each selected residue in the metallocarboxypeptidase enzyme and the nearest heavy (non-hydrogen) atom in the bound inhibitor molecule was computed and reported in the “Distances” column in Table 1 (in parentheses, designations of heavy atoms in the PDB structures are indicated). All of the distances measured are less than 9.0 Å, suggesting that rationally designed mutations at these residue positions would enable re-engineering the selected metallocarboxypeptidase enzyme scaffolds to bind specifically to the tethered coupler-amino acid complex T₁R₁ moiety.

TABLE 1
Analyzed metallocarboxypeptidase enzyme Protein Data Bank (PDB)
accession codes (PDB ID), metallocarboxypeptidase family, organismal origin of the
metallocarboxypeptidase enzyme, residues selected for rationally designed mutations for
engineering specific binding to a tethered coupler-amino acid complex T1R1 moiety,
distances between the Cα atom of each selected residue in the metallocarboxypeptidase
enzyme and the nearest heavy (non-hydrogen) atom in the bound inhibitor molecule (atom
name in parentheses), and the wild-type enzyme sequence used during structural analysis.

| | | | Distances | | |
|---|---|---|---|---|---|
| | | | (Nearest Ligand | | |
| | | Residues | Heavy Atom to | | |
| PDB ID/ | | to be | Residue Cα | Inhibitor | |
| Family | Organism | engineered | Atom) (Å) | Molecule | Protein Sequence |
| 1CBX/ | Bos | D142, N144, | 7.654 (O3), | L- | ARSTNTFNYATYHTLDEIYDFMDLLVA |
| CPA; | taurus | R145, T164, | 4.799 (O3), | benzylsuccinic | EHPQLVSKLQIGRSYEGRPIYVLKFSTG |
| SEQ | | L203, I243, | 6.611 (O3), | acid | GSNRPAIWIDLGIHSREWITQATGVWFA |
| ID | | I247, Y248, | 7.64 (O4), | | KKFTENYGQNPSFTAILDSMDIFLEIVTN |
| NO: | | A250, G253, | 6.562 (CE2), | | PNGFAFTHSENRLWRKTRSVTSSSLCVG |
| 14 | | S254, I255, | 6.079 (CD2), | | VDANRNWDAGFGKAGASSSPCSETYH |
| | | D256, T268, | 6.472 (CB), | | GKYANSEVEVKSIVDFVKNHGNFKAFL |
| | | E270 | 7.101 (CB), | | SIHSYSQLLLYPYGYTTQSIPDKTELNQ |
| | | | 5.226 (CD1), | | VAKSAVAALKSLYGTSYKYGSIITTIYQ |
| | | | 4.056 (CE1), | | ASGGSIDWSYNQGIKYSFTFELRDTGRY |
| | | | 5.715 (CZ), | | GFLLPASQIIPTAQETWLGVLTIMEHTV |
| | | | 5.634 (CZ), | | NN |
| | | | 6.942 (CZ), | | |
| | | | 5.375 (CE2), | | |
| | | | 5.564 (O2) | | |
| 7CPA/ | Bos | D142, N144, | 7.849 (O11), | O-((((N- | ARSTNTFNYATYHTLDEIYDFMDLLVA |
| CPA; | taurus | R145, T164, | 5.086 (O11), | phenyl- | QHPELVSKLQIGRSYEGRPIYVLKFSTG |
| SEQ | | L203, G207, | 6.999 (O11), | methoxy- | GSNRPAIWIDLGIHSREWITQATGVWFA |
| ID | | I243, I247, | 5.688 (CDL), | carbonyl)- | KKFTENYGQNPSFTAILDSMDIFLEIVTN |
| NO: | | Y248, A250, | 6.935 (CE5), | phenylalanyl- | PNGFAFTHSENRLWRKTRSVTSSSLCVG |
| 15 | | G253, S254, | 5.686 (CE6), | carbonyl) | VDANRNWDAGFGKAGASSSPCSETYH |
| | | I255, D256, | 6.149 (CE6), | amino)- | GKYANSEVEVKSIVDFVKNHGNFKAFL |
| | | T268, E270 | 5.929 (CZ1), | isobutyl) | SIHSYSQLLLYPYGYTTQSIPDKTELNQ |
| | | | 7.08 (CEB), | hydroxy | VAKSAVAALKSLYGTSYKYGSIITTIYQ |
| | | | 5.036 (CE6), | phosphinyl)- | ASGGSIDWSYNQGIKYSFTFELRDTGRY |
| | | | 4.332 (CZ4), | 3- | GFLLPASQIIPTAQETWLGVLTIMEHTV |
| | | | 6.205 (CZ4), | phenylacetic | NN |
| | | | 5.978 (CZ4), | acid | |
| | | | 7.13 (CZ4), | | |
| | | | 5.563 (CE5), | | |
| | | | 5.564 (OPW) | | |
| 1ZG8/ | Sus | D138, N140, | 7.888 (O14), | (2R)-2-(3- | TGHSYEKYNNWETIEAWTKQVTSENPD |
| CPB; | scrofa | R141, L200, | 4.677 (O14), | {[amino | LISRTAIGTTFLGNNIYLLKVGKPGPNKP |
| SEQ | | S204, G240, | 6.84 (N12), | (imino) | AIFMDCGFHAREWISHAFCQWFVREAV |
| ID | | I244, Y245, | 5.658 (C7), | methyl]amino} | LTYGYESHMTEFLNKLDFYVLPVLNID |
| NO: | | A247, A248, | 4.537 (N11), | phenyl)- | GYIYTWTKNRMWRKTRSTNAGTTCIGT |
| 16 | | G250, D252, | 5.178 (C8), | 3- | DPNRNFDAGWCTTGASTDPCDETYCGS |
| | | D253, T265, | 6.35 (C7), | sulfanylpropanoic | AAESEKETKALADFIRNNLSSIKAYLTIH |
| | | E267 | 7.136 (N12), | acid | SYSQMILYPYSYDYKLPENNAELNNLA |
| | | | 4.478 (N12), | | KAAVKELATLYGTKYTYGPGATTIYPA |
| | | | 5.777 (N11), | | AGGSDDWAYDQGIKYSFTFELRDKGRY |
| | | | 3.333 (N11), | | GFILPESQIQATCEETMLAIKYVTNYVL |
| | | | 5.754 (N11), | | GHL |
| | | | 5.641 (N11), | | |
| | | | 5.33 (C8), | | |
| | | | 6.383 (C6) | | |
| 1ZG9/ | Sus | D138, N140, | 7.859 (O11), | 5- | TGHSYEKYNNWETIEAWTKQVTSENPD |
| CPB; | scrofa | R141, T160, | 4.766 (O11), | {[amino | LISRTAIGTTFLGNNIYLLKVGKPGPNKP |
| SEQ | | L200, S204, | 6.788 (O11), | (imino) | AIFMDCGFHAREWISHAFCQWFVREAV |
| ID | | G240, I244, | 7.567 (O12), | methyl]amino}- | LTYGYESHMTEFLNKLDFYVLPVLNID |
| NO: | | Y245, A247, | 6.051 (C5), | 2- | GYIYTWTKNRMWRKTRSTNAGTTCIGT |
| 17 | | G250, S251, | 4.807 (N8), | (sulfanyl | DPNRNFDAGWCTTGASTDPCDETYCGS |
| | | D252, D253, | 5.63 (C5), | methyl) | AAESEKETKALADFIRNNLSSIKAYLTIH |
| | | T265, E267 | 6.763 (C3), | pentanoic | SYSQMILYPYSYDYKLPENNAELNNLA |
| | | | 6.98 (N9), | acid | KAAVKELATLYGTKYTYGPGATTIYPA |
| | | | 4.696 (N9), | | AGGSDDWAYDQGIKYSFTFELRDKGRY |
| | | | 3.421 (N8), | | GFILPESQIQATCEETMLAIKYVTNYVL |
| | | | 6.244 (N8), | | GHL |
| | | | 5.825 (N8), | | |
| | | | 5.845 (N8), | | |
| | | | 5.387 (C5), | | |
| | | | 6.757 (C1) | | |
| 1H8L/ | Lophonetta | R132, D139, | 8.106 (O12), | 2- | QAVQPVDFRHHHFSDMEIFLRRYANEY |
| CPD; | specularioides | N141, R142, | 7.914 (O15), | guanidino | PSITRLYSVGKSVELRELYVMEISDNPGI |
| SEQ | | G179, N185, | 5.286 (O15), | ethylmercapto) | HEAGEPEFKYIGNMHGNEVVGRELLLN |
| ID | | D189, G243, | 6.77 (O15), | succinic | LIEYLCKNFGTDPEVTDLVQSTRIHIMPS |
| NO: | | A244, W246, | 5.062 (O13), | acid | MNPDGYEKSQEGDRGGTVGRNNSNNY |
| 18 | | Y247, N248, | 5.686 (C5), | | DLNRNFPDQFFQVTDPPQPETLAVMSW |
| | | V249, Q254, | 3.863 (N2), | | LKTYPFVLSANLHGGSLVVNYPFDDDE |
| | | T267, E269 | 3.428 (N2), | | QGIAIYSKSPDDAVFQQLALSYSKENKK |
| | | | 5.021 (N2), | | MYQGSPCKDLYPTEYFPHGITNGAQWY |
| | | | 4.875 (N4), | | NVPGGMQDWNYLNTNCFEVTIELGCV |
| | | | 3.883 (N4), | | KYPKAEELPKYWEQNRRSLLQFIKQVH |
| | | | 5.039 (N4), | | RGIWGFVLDATDGRGILNATISVADINH |
| | | | 4.726 (N2), | | PVTTYKDGDYWRLLVQGTYKVTASAR |
| | | | 8.365 (C5), | | GYDPVTKTVEVDSKGGVQVNFTLSRT |
| | | | 6.067 (C6), | | |
| | | | 6.375 (O13) | | |
| 3V7Z/ | Thermoactinomyces | D144, N146, | 7.928 (O14), | (2- | DFPSYDSGYHNYNEMVNKINTVASNYP |
| CPT; | vulgaris | R147, L211, | 5.056 (O14), | guanidino | NIVKKFSIGKSYEGRELWAVKISDNVGT |
| SEQ | | G215, A251, | 6.985 (O14), | ethylmercapto) | DENEPEVLYTALHHAREHLTVEMALYT |
| ID | | S252, L254, | 6.848 (C5), | succinic | LDLFTQNYNLDSRITNLVNNREIYIVFNI |
| NO: | | Y255, I256, | 3.627 (N4), | acid | NPDGGEYDISSGSYKSWRKNRQPNSGS |
| 19 | | T257, D260, | 4.097 (N4), | | SYVGTDLNRNYGYKWGCCGGSSGSPSS |
| | | T275, E277 | 4.285 (N4), | | ETYRGRSAFSAPETAAMRDFINSRVVG |
| | | | 4.975 (N2), | | GKQQIKTLITFHTYSELILYPYGYTYTD |
| | | | 4.567 (N2), | | VPSDMTQDDFNVFKTMANTMAQTNGY |
| | | | 4.464 (N4), | | TPQQASDLYITDGDMTDWAYGQHKIFA |
| | | | 4.151 (N4), | | FTFEMYPTSYNPGFYPPDEVIGRETSRN |
| | | | 6.356 (C6), | | KEAVLYVAEKADCPYSVIGKSC |
| | | | 6.422 (S7), | | |
| | | | 6.034 (O13) | | |
| 4DUK/ | Thermoactinomyces | D144, N146, | 8.076 (O4), | L- | DFPSYDSGYHNYNEMVNKINTVASNYP |
| CPT; | vulgaris | R147, L211, | 5.167 (O4), | benzylsuccinic | NIVKKFSIGKSYEGRELWAVKISDNVGT |
| SEQ | | G215, A251, | 7.107 (O4), | acid | DENEPEVLYTALHHAREHLTVEMALYT |
| ID | | S252, L254, | 6.898 (CZ), | | LDLFTQNYNLDSRITNLVNNREIYIVFNI |
| NO: | | Y255, T257, | 4.733 (CZ), | | NPDGGEYDISSGSYKSWRKNRQPNSGS |
| 20 | | D260, T262, | 4.193 (CE2), | | SYVGTDLNRNYGYKWGCCGGSSGSPSS |
| | | T275, E277 | 5.663 (CE2), | | ETYRGRSAFSAPETAAMRDFINSRVVG |
| | | | 5.909 (CD2), | | GKQQIKTLITFHTYSELILYPYGYTYTD |
| | | | 5.865 (CD2), | | VPSDMTQDDFNVFKTMANTMAQTNGY |
| | | | 5.035 (CE2), | | TPQQASDLYITDGDMTDWAYGQHKIFA |
| | | | 5.642 (CE1), | | FTFEMYPTSYNPGFYPPDEVIGRETSRN |
| | | | 6.71 (CE1), | | KEAVLYVAEKADCPYSVIGKSC |
| | | | 6.151 (CE1), | | |
| | | | 5.997 (O2) | | |
| 3D67/ | Homo | V35, D232, | 6.504 (O15), | (2- | AQSGQVLAALPRTSRQVQVLQNLTTTY |
| CPU; | sapiens | N234, R235, | 8.019 (O14), | guanidino | EIVLWQPVTADLIVKKKQVHFFVNASD |
| SEQ | | V295, P297, | 5.329 (O14), | ethylmercapto) | VDNVKAHLNVSGIPCSVLLADVEDLIQ |
| ID | | S299, G336, | 7.157 (N2), | succinic | QQISNDTVSPRASASYYEQYHSLNEIYS |
| NO: | | L340, Y341, | 6.881 (N4), | acid | WIEFITERHPDMLTKIHIGSSFEKYPLYV |
| 21 | | A343, G346, | 5.898 (N4), | | LKVSGKEQTAKNAIWIDCGIHAREWISP |
| | | D348, D349, | 4.678 (N4), | | AFCLWFIGHITQFYGIIGQYTNLLRLVDF |
| | | T361, E363 | 5.223 (N4), | | YVMPVVNVDGYDYSWKKNRMWRKN |
| | | | 6.459 (C5), | | RSFYANNHCIGTDLNRNFASKHWCEEG |
| | | | 6.848 (N3), | | ASSSSCSETYCGLYPESEPEVKAVASFL |
| | | | 5.279 (N3), | | RRNINQIKAYISMHSYSQHIVFPYSYTRS |
| | | | 3.915 (N2), | | KSKDHEELSLVASEAVRAIEKTSKNTRY |
| | | | 5.458 (N4), | | THGHGSETLYLAPGGGDDWIYDLGIKY |
| | | | 5.966 (N4), | | SFTIELRDTGTYGFLLPERYIKPTCREAF |
| | | | 6.378 (C6), | | AAVSKIAWHVIRNV |
| | | | 5.635 (O12) | | |

### Example 7. Engineering and Maturation of Binding Agents Capable of Specific Binding to the Coupler-Amino Acid Complex

Engineering and maturation of binding agents involves improving affinities of potential binding sites through rational, structure-based approaches on a parental scaffold and generating libraries that contain degenerate NNK codons at multiple, defined positions using Kunkel mutagenesis and phage display selection. Kunkel mutagenesis is a known site-directed mutagenesis strategy that introduces point mutations by annealing mutation-containing oligonucleotides to single-stranded uracil-containing single strand DNA (dU-ssDNA) templates. Exemplary Kunkel mutagenesis and phage display selection methods are described in U.S. Pat. No. 9,102,711 B2; U.S. Ser. No. 10/906,968 B2; and Kunkel, Proc. Natl. Acad. Sci. USA, 1985, 83(2):488-492.

In this example, high diversity (˜10¹⁰) phage libraries using NNK variant site encoding were constructed targeting residues positions within the substrate-binding pockets of the selected scaffolds disclosed in Example 6. Phosphorylated primers were obtained that possess degenerate codons at intended positions and are annealed to uracilated ssDNA containing the parental sequence of the same binding agent of interest with introduced SacII sites. After polymerase extension and ligation, the heteroduplex DNA was transformed into custom TG1RM cells (Lucigen TG1 Electrocompetent Cells containing a pCDF-1b plasmid expressing SacII enzyme), which removed undesired template DNA with SacII sites resulting in 10⁹-10¹⁰ libraries. Monovalent phage libraries were packaged using standard helper phage and precipitated using PEG/NaCl solution. Using standard protocols, phage libraries were panned against different immobilized coupler-amino acid complexes.

For each round of phage display selection, precipitated phage in the presence of amino acid competitors were first depleted against beads coated with off-target ocupler-amino acid complexes for 1 hour at 24° C. and then panned against beads coated with target coupler-amino acid complexes for 1 hour at 24° C. After washing 6 times with PBST, beads-bound phages were eluted using 0.2 M pH=2.2 glycine for 10 min at 24° C. and then subsequently used to infect mid-log phase TG1 cells. Once the final round of selection was complete, the output was profiled in a phage-based, multiplexed binding assay (Luminex, DiaSorin, USA) against a panel of coupler-amino acid complexes and underwent next-generation sequencing to obtain clone enrichment sequence information. Luminex enables analysis of binding of phage libraries against multiple coupler-amino acid complex targets immobilized on beads in a single assay well. This is accomplished by spatially separating immunoassays performed on beads that contain unique fluorophore cores that exhibit distinct excitation/emission profiles. Multiple target amino acid-specific beads are combined in a single well of a multi-well microplate to detect and quantify multiple targets simultaneously. Specific binders are isolated against a variety of coupler-amino acid complexes. Based on the sequence identities after enrichment, consensus mutations or mutational hotspots are identified.

Binder maturation for affinity and specificity involves multiple cycles of error prone PCR prior to library construction via Kunkel mutagenesis and phage display selection, performed essentially as described above. Briefly, 60-90 cycles of error prone PCR on a parental binder generate PCR amplicons with an average of 4-6 random amino acid mutations per 100 amino acids. The dsDNA amplicon is digested by lambda exonuclease into “megaprimer” ssDNA, which is used to generate heteroduplex DNA by annealing to uracilated ssDNA of the vector containing the parental sequence of the same binder of interest with introduced SacII sites. After polymerase extension and ligation, the heteroduplex DNA is transformed into custom TG1RM cells (Lucigen TG1 Electrocompetent Cells containing a pCDF-1b plasmid expressing SacII enzyme), which removed undesired template DNA with SacII sites resulting in 10⁹-10¹⁰ libraries. Monovalent phage libraries are packaged using standard helper phage and precipitated using PEG/NaCl solution. For each round of phage display selection, precipitated phage in the presence of peptide and protein competitors are first depleted against beads coated with off-target coupler-amino acid complexes for 1 hour at 24° C. and then panned against beads coated with target coupler-amino acid complexes for 1 hour at 24° C.

Engineered binders conjugated with an N-terminal hexa-histidine tag are expressed and purified according to standard methods. Plasmids are transformed into chemically competent E. coli cells. After recovery in warm SOC, 80 ul of transformed culture is added to 1 ml 2YT containing corresponding antibiotic. The culture is grown overnight for ˜20 hours at 37° C. This culture is subsequently used to inoculate another larger volume culture of 2YT containing corresponding antibiotic at a 100-fold dilution. The culture is grown at 37° C. for 3-4 hours until an optical density of 0.6 is reached. Protein expression is induced with a final concentration of 0.5 mM IPTG at 15° C. The cultures are grown for an additional 16-20 hours, and the cells are harvested and stored at −80° C. until ready for use.

Stored cellular pellets are resuspend in 25 mM Tris pH=7.9, 500 mM NaCl, and 10 mM imidazole with included protease inhibitor and are lysed by sonication. The clarified lysate is loaded onto an AKTA FPLC using a tandem purification method of nickel affinity and size-exclusion chromatography. The retained protein is eluted from the nickel affinity column using 25 mM Tris pH 7.9, 500 mM NaCl, 300 mM imidazole directly onto the size-exclusion column. The size-exclusion buffer is 25 mM PO4 pH 7.4 with 150 mM NaCl, and after elution and concentration, proteins were aliquoted in 10% glycerol, frozen, and stored at −80° C.

### Example 8. Use of Error Prone Library and SSM Library of S46 Dipeptidyl Peptidases to Evolve for a Modified Dipeptidyl Peptidase (Engineered Cleavase Enzyme)

To engineer modified dipeptidyl peptidases that is configured to cleave the coupler-amino acid complex from the tethered coupler-polypeptide complex, site saturation mutagenesis and error prone libraries of DAP BII dipeptidyl peptidases are created as disclosed in patent application U.S. Ser. No. 17/213,169 and PCT/US2021/023347. The variant libraries are transformed into an arginine auxotroph strain of E. coli, which has a deletion in the argA gene (strain JW2786-1). Genetic selection is performed on the transformed E. coli using M9 minimal media agar plates supplemented with arginine N-terminal modified peptides. In these peptides, the N-terminal modification is comprised of a base moiety conjugating to the alpha amine of the N-terminus, a linker moiety, and an coupling moiety capable of tethering the modified N-terminus to a proximal anchor. Several agents are screened including those that was used to label the N-terminal of arginine-containing peptides. The plates are incubated at 35° C. until colonies appear. From the surviving cells, plasmid DNA is subsequently isolated and sequenced to identify the mutations that generate an active modified dipeptidyl cleavase that recognizes labeled NTAAs and tolerates a longer linker at the substrate binding pockets. After sequence verification, these engineered dipeptidyl peptidases can be expressed, purified and tested against soluble and tethered substrates. After verification of these clones, novel mutations that are identified can be combined to create new libraries to further improve the performance of modified dipeptidyl peptidases.

### Example 9. Starting Cleavase Scaffolds from Other DPP and Aminopeptidase Families

S46 family of dipeptidyl peptidases has an extra capping domain that can potentially limit the catalytic activity on tethered substrate. This limitation is mitigated by using couplers with a long linker (such as a PEG linker) for tethering of the coupler-polypeptide complex. Instead of a DAP BII scaffold, additional scaffolds can be used to engineer modified dipeptidyl peptidases that is configured to cleave the coupler-amino acid complex from the tethered coupler-polypeptide complex, for example, DPP5 and iminopeptidase. These families of enzymes have a different capping domain (different secondary structure and/or size), and are preferable to allow easier access of tethered substrates based on structural modeling. A sequential two-step enzyme engineering approach can be employed similar to the approach for the S46 family disclosed in Example 8 and in patent application U.S. Ser. No. 17/213,169 and PCT/US2021/023347. In the first step, the genetic selection platform to evolve native peptidases to modified peptidases can be used, and in the second step, mutations can be introduced around of the substrate binding site of the peptidases in areas that are involved in recognition of the coupler moiety (“mod pocket”) and the terminal amino acid (“S2 pocket”) to create tolerance for tethered substrates.

### Example 10. Engineered Dipeptide Cleavases can Remove Single Labeled NTAAs of a Model Polypeptide

A set of dipeptide cleavase enzymes was evolved from an S46 DPP library as described in Example 8 to recognize and cleave a modified NTAA using M15-L-P1 target polypeptides (polypeptide sequences: M15-L-P1-AR, where M15 is a 2-aminobenzamide, P1 is one of the 17 natural amino acids, excluding C, K, R) and the dipeptide cleavase scaffold from Thermomonas hydrothermalis (SEQ ID NO: 7). The enzymes can efficiently cleave M15-L-labeled polypeptides between P1 and P2 amino acid residues (the P2 residue is alanine), thus are configured to remove a single labeled terminal amino acid from the polypeptide (FIG. 7A and FIG. 7B). To accommodate the M15-L label in the substrate binding site, all modified dipeptide cleavases contained the following mutations at the conserved residues that form an amine binding site in unmodified dipeptidyl aminopeptidases: N214M, W215G, R219T, N329R, D673A (the indicated residue numbers correspond to positions of SEQ ID NO: 7). The cleavage efficiency of the evolved enzymes depended on the nature of the P1 residue.

Each evolved cleavase was individually assayed on all M15-L-P1 target polypeptides. In this assay, an individual cleavase clone was expressed and purified, and then incubated with each peptide substrate for 3 hours at 52° C. Six μM enzyme in 5 mM phosphate buffer at pH 8 were used. The UV absorbance of both product and starting material in the final reaction was measured on HPLC and converted to percentage of conversion (FIG. 7A). M15-L-P-AR exhibited poor cleavage efficiency with the set of seven Cleavase clones, but further directed evolution can be used to address this issue. Additionally, efficiency of cleavage reactions were assessed on peptide-DNA conjugates. In this assay, peptide substrates were modified to have an azide group at the C-terminal lysine that was linked to dibenzocyclooctyne (DBCO)-activated PEG12 linker connected with a DNA oligo. M15-L-P1-GAEIAGDVAGGK peptides were used (SEQ ID NO: 11), and for D and N as P1, the Gly residue at P2 position was replaced with Val. In FIG. 7B, the cleavage events were monitored by UREA-PAGE assay. It was found that the first selected modified cleavase (M15-L_Z001) provided 100% cleavage for polypeptides with the following M15-L-labeled P1 residues: A, I, L, M, Q, V. Other selected modified cleavases provided 80-100% cleavage for polypeptides with the following groups of M15-L-labeled P1 residues: D, E; S, T; G; N; H, Y; F, W. A broad cleavage of a single labeled terminal amino acid from the polypeptide can be achieved by combining two or more dipeptide cleavases in a set. For example, as shown in FIG. 7A and FIG. 7B, a set of 7 selected dipeptide cleavases can provide broad activity for removal of almost all M15-L-labeled P1 residues from the polypeptide. In another example, a set of two modified dipeptide cleavases can also cleave the majority of M15-L-labeled P1 residues from the polypeptide, except for F, G, H, P, W residues (FIG. 7C). In this assay, short peptides with M15-L-P1-AR sequence are used, same as in FIG. 7A. Other cleavase combinations can be created to achieve a desired level of cleavage specificity, such as different sets of two, three, four or more enzymes.

### Example 11. Engineered Dipeptide Cleavases can Accommodate a Substrate Peptide Modified for Tethering to a Solid Support

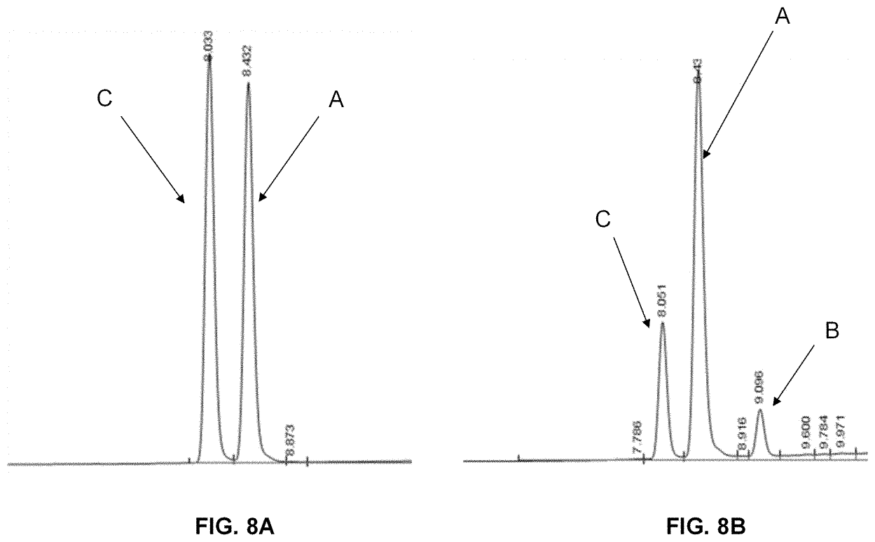

An exemplary cleavage reaction was performed to evaluate tolerance of a engineered dipeptidyl peptidase clone to a modified NTM group (M15- K(biotin)) attached to a model peptide AAR. A engineered dipeptidyl peptidase clone (SEQ ID NO: 6) derived from dipeptidyl peptidase from Thermomonas hydrothermalis (SEQ ID NO: 7) was expressed and purified, and then 2 uM of the engineered dipeptidyl peptidase was incubated with 300 uM of M15-K(biotin)-AAR (SEQ ID NO: 12) peptide for 2 hour at 52° C. in 5 mM phosphate buffer at pH 8. The UV absorbance at OD=254 nm of both starting material (FIG. 8A) and reaction product (FIG. 8B, after incubation with the peptidase) was measured on HPLC and converted to percentage of conversion. In FIG. 8A-FIG. 8B, A designates a signal from M15-K(biotin)-AAR peptide; B designates a signal from M15-K(biotin)-A molecule, and C designates a signal from a control peptide. After incubation with the peptidase for 1 h, about 10% of the model peptide was converted to the cleavage product, M15-K(biotin)-A (FIG. 8B). This indicates that an engineered dipeptide cleavase can accommodate a substrate peptide modified for tethering to a solid support. For example, biotin group can be substituted for a PEG or aliphatic linker that can be attached to the solid support. An exemplar NTM is M15-(azido-PEG4)-L-Lysine or M15-6-Azido-L-lysine (BaseClick) wherein the azido can be coupled to the solid support using either CUAAC or SPAAC click chemistry. The M15-(azido-PEG4)-L-Lysine can be installed in a two-step process using N-Fmoc-N′-(azido-PEG4)-L-Lysine-PFP (BroadPharm) ester to couple to the NTAA and then M15-NHS or M15-PFP to couple to the installed azido-PEG4-lysine residue after removal of the Fmoc moiety (via 20% piperidine-DMF for 10 min).

The present disclosure is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for identifying a polypeptide, comprising the steps of:
(a) providing a polypeptide and an associated recording tag attached to a solid support;
(b) contacting the polypeptide with a coupler, wherein the coupler binds to a terminal amino acid of the polypeptide to form a coupler-polypeptide complex;
(c) attaching the coupler to the solid support;
(d) cleaving the peptide bond between the terminal amino acid and the penultimate terminal amino acid of the polypeptide within the coupler-polypeptide complex, thereby exposing a new terminal amino acid of the polypeptide and generating a coupler-amino acid complex attached to the solid support;
(e) contacting the coupler-amino acid complex attached to the solid support with a binding agent capable of specific binding to the coupler-amino acid complex, wherein the binding agent comprises a coding tag with identifying information regarding the binding agent;
(f) transferring the information of the coding tag of the binding agent to the recording tag to generate an extended recording tag;
(g) releasing the coupler-amino acid complex from the solid support;
(h) repeating steps (b) through (g) at least one more time; and
(i) analyzing the extended recording tag after information transfer, thereby identifying at least a portion of the sequence of the polypeptide.

2. The method of claim 1, wherein the terminal amino acid is modified to produce a modified terminal amino acid before contacting the polypeptide with the coupler; at step (b) the coupler binds to the modified terminal amino acid; at step (d) a coupler-modified amino acid complex attached to the solid support is generated after cleavage of the peptide bond between the terminal amino acid and the penultimate terminal amino acid of the polypeptide; and at step (e) the binding agent is capable of binding to the coupler-modified amino acid complex.

3. The method of claim 1, wherein the coupler is releasably attached to the solid support.

4. The method of claim 1, wherein the polypeptide is further associated with a first stabilizing component and the coupler comprises a second stabilizing component; at step (c) after binding of the coupler to the terminal amino acid of the polypeptide, the first and second stabilizing components are linked together to form a tethering complex that comprises a first stabilizing component attached to the solid support and the second stabilizing component linked to the coupler-polypeptide complex; and at step (d) the coupler-amino acid complex generated after cleavage of the peptide bond between the terminal amino acid and the penultimate terminal amino acid of the polypeptide is releasably attached to the solid support via the tethering complex.

5. The method of claim 4, wherein the first stabilizing component is releasably associated with the polypeptide attached to the solid support.

6. The method of claim 4, wherein the stabilizing components are linked upon introduction of a linking agent that binds to the first stabilizing component and to the second stabilizing component.

7. The method of claim 6, wherein the linking agent is releasably associated with the first stabilizing component.

8. The method of claim 6, wherein the linking agent activates a stabilizing component.

9. The method of claim 6, wherein the linking agent comprises a linking polypeptide that binds to the first stabilizing component and to the second stabilizing component.

10. The method of claim 4, wherein the first or second stabilizing component comprises a polynucleotide; the stabilizing components are linked upon introduction of a linking agent that hybridizes to the polynucleotide of one of the stabilizing components.

11. The method of claim 4, wherein the first stabilizing component is the same as the second stabilizing component.

12. The method of claim 4, wherein the stabilizing components are linked upon introduction of a linking agent, the linking agent binds to the first stabilizing component and to the second stabilizing component, and the first stabilizing component has a lower affinity to the linking agent in comparison to an affinity of the second stabilizing component to the linking agent.

13. The method of claim 1, wherein the binding agent binds to the coupler-amino acid complex, but does not bind to the corresponding amino acid separated from the coupler-amino acid complex, or affinity of the binding agent for the corresponding amino acid separated from the coupler-amino acid complex is reduced compared to affinity of the binding agent for the coupler-amino acid complex by at least an order of magnitude.

14. The method of claim 1, wherein the extended recording tag after information transfer is analyzed using a nucleic acid sequencing method.

15. The method of claim 1, wherein the binding agent comprises an engineered carboxypeptidase.

16. The method of claim 1, wherein the coupler binds to a N-terminal amino acid (NTAA) of the polypeptide.

17. The method of claim 1, which further comprises releasing the coupler-amino acid complex from the solid support after transferring the information of the coding tag to the recording tag.

18. A method for identifying a terminal amino acid of a polypeptide, comprising the steps of:
(a) providing a polypeptide and an associated recording tag attached to a solid support;
(b) contacting the polypeptide with a coupler, wherein the coupler binds to a terminal amino acid of the polypeptide to form a coupler-polypeptide complex;
(c) attaching the coupler to the solid support;
(d) cleaving the peptide bond between the terminal amino acid and the penultimate terminal amino acid of the polypeptide within the coupler-polypeptide complex to generate a coupler-amino acid complex attached to the solid support;
(e) contacting the coupler-amino acid complex with a binding agent capable of binding selectively to the coupler-amino acid complex, wherein the binding agent comprises a coding tag with identifying information regarding the binding agent;
(f) transferring the information of the coding tag of the binding agent to the recording tag to generate an extended recording tag; and
(g) analyzing the extended recording tag, thereby identifying the terminal amino acid of the polypeptide.

19. A kit for analyzing a polypeptide, comprising:
a coupler, wherein the coupler is configured to bind to a terminal amino acid of the polypeptide to form a coupler-polypeptide complex;
a reagent for cleaving the peptide bond between the terminal amino acid and the penultimate terminal amino acid of the polypeptide within the coupler-polypeptide complex to generate a coupler-amino acid complex; and
a binding agent capable of binding to the coupler-amino acid complex.


SEQUENCE LISTING (21 sequences)

SEQ ID NO: 1 - protein, 309 aa, Bos taurus
  Carboxypeptidase A1
ARSTNTFNYATYHTLDEIYDFMDLLVAEHPQLVSKLQIGRSYEGRPIYVLKFSTGGSNRPAIWIDLGIHSREWITQATGVWFAKKFTEDYGQDPSFTAILDSMDIFLEIVTNPDGFAFTHSQNRLWRKTRSVTSSSLCVGVDANRNWDAGFGKAGASSSPCSETYHGKYANSEVEVKSIVDFVKDHGNFKAFLSIHSYSQLLLYPYGYTTQSIPDKTELNQVAKSAVEALKSLYGTSYKYGSIITTIYQASGGSIDWSYNQGIKYSFTFELRDTGRYGFLLPASQIIPTAQETWLGVLTIMEHTLNNLY

SEQ ID NO: 2 - protein, 309 aa, Homo sapiens
  Carboxypeptidase A1
ARSTDTFNYATYHTLEEIYDFLDLLVAENPHLVSKIQIGNTYEGRPIYVLKFSTGGSKRPAIWIDTGIHSREWVTQASGVWFAKKITQDYGQDAAFTAILDTLDIFLEIVTNPDGFAFTHSTNRMWRKTRSHTAGSLCIGVDPNRNWDAGFGLSGASSNPCSETYHGKFANSEVEVKSIVDFVKDHGNIKAFISIHSYSQLLMYPYGYKTEPVPDQDELDQLSKAAVTALASLYGTKFNYGSIIKAIYQASGSTIDWTYSQGIKYSFTFELRDTGRYGFLLPASQIIPTAKETWLALLTIMEHTLNHPY

SEQ ID NO: 3 - protein, 334 aa, Paludibacter
  Carboxypeptidase; UniProt A0A235B6N3
QMMPMDFPPGDSEYHNYDEMVAEIQRTAREHPDLVKTFSIGQSYEGRELWAAKISDNPDRDEDEPEVLFVGQHHAREHLTVEMALYILKLYTDSYGSDSRITDLVDSREIFVVFSVNPDGSEYDIRDDSYSYWRKNRQPNGGSRYIGTDLNRNFGYKWGCCGGSSGWPSSETYRGDSPFSAPETARLRDFIQSRVVDGEQQIKTAITFHTYSELILWPYGYTYDDTPSDMSADDYAVFETMGRAMGRTNGYTAQQSSDLYITDGDMVDWAYGDQGIFAFTFEMYPKTSNPGFYPPDEVIDRETSRNREAVLYLTEQAECPYRTIGKEGQYCQAE

SEQ ID NO: 4 - protein, 331 aa, Thermoactinomyces vulgaris
  Carboxypeptidase T
SLTTQDFPSYDSGYHNYNEMVNKINTVASNYPNIVKKFSIGKSYEGRELWAVKISDNVGTDENEPEVLYTALHHAREHLTVEMALYTLDLFTQNYNLDSRITNLVNNREIYIVFNINPDGGEYDISSGSYKSWRKNRQPNSGSSYVGTDLNRNYGYKWGCCGGSSGSPSSETYRGRSAFSAPETAAMRDFINSRVVGGKQQIKTLITFHTYSELILYPYGYTYTDVPSDMTQDDFNVFKTMANTMAQTNGYTPQQASDLYITDGDMTDWAYGQHKIFAFTFEMYPTSYNPGFYPPDEVIGRETSRNKEAVLYVAEKADCPYSVIGKSCSTK

SEQ ID NO: 5 - protein, 334 aa, Thermoactinomycetaceae
  Zinc carboxypeptidase; UniProt A0A1H8BS52
FFGAEDFPSYDSLYHNYQEMVAEIDQVAAARPNIVKKFSIGTSYEGRQLWAVKISDQVETDENEPEVLYVSLHHAREHLTVEMALYLLHLFADNYGRDSRITNIVNSREVYIVFNLNPDGGEYDTANGSYQYWRKNRQPNSGSRYIGTDLNRNYGYKWGCCGGSSGSPSSETYRGSAPFSAPETARLRDFINSRVIGGKQQIKASITFHTYSELILWPYGYTYEDTPGDMSADDHQVFVTMGQAMAQTNGYTPQQSSDLYITDGDFTDWAYGAHKIFAFTFEKYPTSSNPGFYPPDEVIARETARNREAVLYLAENADCPYRTIGKGSQYCSAP

SEQ ID NO: 6 - protein, 700 aa, Artificial Sequence
  engineered dipeptidyl peptidase clone
DEGMWVPQQLPEIAGALKKAGLKLDPKQLSDLTGDPMGAVVSLGGCTGSFVSPQGLVATNHHCAYGAIQLNSTPEKNLIKDGFNAPTQADELSAGPNARIYVLEGITDVTAQAKAAMAAAGNDPVARANALEAFEKKITSDCEAEPGYRCRVYSFMGGITYRLFKNLEIKDVRLVYAPPSSVGKFGGDIDMGMWPTHTGDFSFYRAYVGKDGKPAPYSKDNVPYRPKHWLKIADTPLGEGDFVMVAGYPGRTDRYALVAEFENTQNWLYPAISKAYKDQIALVEAAAKDNPEIAVKYAAALAGWNRTSKNFDGQLEGFKRNDVLAIKRREEAAVLEWLRARGKAGTPALEAHAALVKLVADTARTQERDLVLGSFNRTGIIGVAVNLYRLAIERQKPDAEREPGYQQRDLPVIEGSLKQMERRYVPAMDRQLRAYWLDRYVALPAAQHVAAVDAWLGGSDKAAAEAALARLDQSRLGSLEERLKWFNADRAAFEASTDPAIQYAVAVMPTLLAMEQQAKTRYGVALEARPRYLQAVVDYKKSKGQAVYPDANSTLRITYGHVKGYTGLNGKVYTPFTTLEEVAAKETGVEPFDNPKALLEAVAAKRYAGLADARLGTVPVNFLADLDITGGNSGSPVLDANGRLVGLAFAVTLESVASNWVFDPVLTRMISVDQRYMRWIMQEVMPAPQLLEELGVPPRQ

SEQ ID NO: 7 - protein, 723 aa, Thermomonas hydrothermalis
  Dipeptidyl peptidase with the signal peptide
MHKTRLVAALAAALATLAPAAWADEGMWVPQQLPEIAGALKKAGLKLDPKQLSDLTGDPMGAVVSLGGCTGSFVSPQGLVATNHHCAYGAIQLNSTPEKNLIKDGFNAPTQADELSAGPNARIYVLEGITDVTAQAKAAMAAAGNDPVARANALEAFEKKITSDCEAEPGYRCRVYSFMGGITYRLFKNLEIKDVRLVYAPPSSVGKFGGDIDNWMWPRHTGDFSFYRAYVGKDGKPAPYSKDNVPYRPKHWLKIADTPLGEGDFVMVAGYPGRTDRYALVAEFENTQNWLYPAISKAYKDQIALVEAAAKDNPEIAVKYAAALAGWNNTSKNFDGQLEGFKRNDVLAIKRREEAAVLEWLRARGKAGTPALEAHAALVKLVADTARTQERDLVLGSFNRTGIIGVAVNLYRLAIERQKPDAEREPGYQQRDLPVIEGSLKQMERRYVPAMDRQLRAYWLDRYVALPAAQHVAAVDAWLGGSDKAAAEAALARLDQSRLGSLEERLKWFNADRAAFEASTDPAIQYAVAVMPTLLAMEQQAKTRYGVALEARPRYLQAVVDYKKSKGQAVYPDANSTLRITYGHVKGYTGLNGKVYTPFTTLEEVAAKETGVEPFDNPKALLEAVAAKRYAGLADARLGTVPVNFLADLDITGGNSGSPVLDANGRLVGLAFDGTLESVASNWVFDPVLTRMISVDQRYMRWIMQEVMPAPQLLEELGVPPRQ

SEQ ID NO: 8 - DNA, 20 bp, Artificial Sequence
  P5 primer
aatgatacggcgaccaccga

SEQ ID NO: 9 - DNA, 24 bp, Artificial Sequence
  P7 primer
caagcagaagacggcatacgagat

SEQ ID NO: 10 - DNA, 52 bp, Artificial Sequence
  Capping oligo
gactggagttcagacgtgtgctcttccgatctttctactcttctcactcagt

SEQ ID NO: 11 - protein, 13 aa, Artificial Sequence
  Test peptide
  MISC_FEATURE (1)..(1): Xaa can be any naturally occurring amino acid
XGAEIAGDVAGGK

SEQ ID NO: 12 - protein, 4 aa, Artificial Sequence
  Test peptide
  MISC_FEATURE (1)..(1): N-terminal biotin modification
KAAR

SEQ ID NO: 13 - DNA, 12 bp, Artificial Sequence
  model LNA sequence
gtgatatgtccg

SEQ ID NO: 14 - protein, 307 aa, Bos taurus
  scaffold
ARSTNTFNYATYHTLDEIYDFMDLLVAEHPQLVSKLQIGRSYEGRPIYVLKFSTGGSNRPAIWIDLGIHSREWITQATGVWFAKKFTENYGQNPSFTAILDSMDIFLEIVTNPNGFAFTHSENRLWRKTRSVTSSSLCVGVDANRNWDAGFGKAGASSSPCSETYHGKYANSEVEVKSIVDFVKNHGNFKAFLSIHSYSQLLLYPYGYTTQSIPDKTELNQVAKSAVAALKSLYGTSYKYGSIITTIYQASGGSIDWSYNQGIKYSFTFELRDTGRYGFLLPASQIIPTAQETWLGVLTIMEHTVNN

SEQ ID NO: 15 - protein, 307 aa, Bos taurus
  scaffold
ARSTNTFNYATYHTLDEIYDFMDLLVAQHPELVSKLQIGRSYEGRPIYVLKFSTGGSNRPAIWIDLGIHSREWITQATGVWFAKKFTENYGQNPSFTAILDSMDIFLEIVTNPNGFAFTHSENRLWRKTRSVTSSSLCVGVDANRNWDAGFGKAGASSSPCSETYHGKYANSEVEVKSIVDFVKNHGNFKAFLSIHSYSQLLLYPYGYTTQSIPDKTELNQVAKSAVAALKSLYGTSYKYGSIITTIYQASGGSIDWSYNQGIKYSFTFELRDTGRYGFLLPASQIIPTAQETWLGVLTIMEHTVNN

SEQ ID NO: 16 - protein, 305 aa, Sus scrofa
  scaffold
TGHSYEKYNNWETIEAWTKQVTSENPDLISRTAIGTTFLGNNIYLLKVGKPGPNKPAIFMDCGFHAREWISHAFCQWFVREAVLTYGYESHMTEFLNKLDFYVLPVLNIDGYIYTWTKNRMWRKTRSTNAGTTCIGTDPNRNFDAGWCTTGASTDPCDETYCGSAAESEKETKALADFIRNNLSSIKAYLTIHSYSQMILYPYSYDYKLPENNAELNNLAKAAVKELATLYGTKYTYGPGATTIYPAAGGSDDWAYDQGIKYSFTFELRDKGRYGFILPESQIQATCEETMLAIKYVTNYVLGHL

SEQ ID NO: 17 - protein, 305 aa, Sus scrofa; identical to SEQ ID NO: 16
  Scaffold

SEQ ID NO: 18 - protein, 380 aa, Lophonetta specularioides specularioides
  Scaffold
QAVQPVDFRHHHFSDMEIFLRRYANEYPSITRLYSVGKSVELRELYVMEISDNPGIHEAGEPEFKYIGNMHGNEVVGRELLLNLIEYLCKNFGTDPEVTDLVQSTRIHIMPSMNPDGYEKSQEGDRGGTVGRNNSNNYDLNRNFPDQFFQVTDPPQPETLAVMSWLKTYPFVLSANLHGGSLVVNYPFDDDEQGIAIYSKSPDDAVFQQLALSYSKENKKMYQGSPCKDLYPTEYFPHGITNGAQWYNVPGGMQDWNYLNTNCFEVTIELGCVKYPKAEELPKYWEQNRRSLLQFIKQVHRGIWGFVLDATDGRGILNATISVADINHPVTTYKDGDYWRLLVQGTYKVTASARGYDPVTKTVEVDSKGGVQVNFTLSRT

SEQ ID NO: 19 - protein, 323 aa, Thermoactinomyces vulgaris [sequence omitted]

SEQ ID NO: 20 - protein, 323 aa, Thermoactinomyces vulgaris [sequence omitted]

SEQ ID NO: 21 - protein, 401 aa, Homo sapiens
  CPU scaffold
AQSGQVLAALPRTSRQVQVLQNLTTTYEIVLWQPVTADLIVKKKQVHFFVNASDVDNVKAHLNVSGIPCSVLLADVEDLIQQQISNDTVSPRASASYYEQYHSLNEIYSWIEFITERHPDMLTKIHIGSSFEKYPLYVLKVSGKEQTAKNAIWIDCGIHAREWISPAFCLWFIGHITQFYGIIGQYTNLLRLVDFYVMPVVNVDGYDYSWKKNRMWRKNRSFYANNHCIGTDLNRNFASKHWCEEGASSSSCSETYCGLYPESEPEVKAVASFLRRNINQIKAYISMHSYSQHIVFPYSYTRSKSKDHEELSLVASEAVRAIEKTSKNTRYTHGHGSETLYLAPGGGDDWIYDLGIKYSFTIELRDTGTYGFLLPERYIKPTCREAFAAVSKIAWHVIRNV